(19) United States
(12) Patent Application Publication
(10) Pub. No.: US 2024/0400834 A1
(43) Pub. Date: Dec. 5, 2024

(54) MULTIMERIC DYE MATERIALS AND DEVICES USING THEM

(71) Applicant: Flexterra, Inc., Skokie, IL (US)

(72) Inventors: Antonio Facchetti, Skokie, IL (US); Zhihua Chen, Skokie, IL (US); Shaofeng Lu, Skokie, IL (US); Yu Xia, Skokie, IL (US); Mark Seger, Skokie, IL (US); Shiuan-Iou Lin, Zhubei City (TW); Hsiu-Chun Wu, Zhubei City (TW); Liang-Je Lai, Zhubei City (TW); Jing-Yun Chen, Zhubei City (TW)

(21) Appl. No.: 18/686,476

(22) PCT Filed: Apr. 21, 2022
(86) PCT No.: PCT/US2022/025790

§ 371 Date: Feb. 26, 2024

Related U.S. Application Data
(60) Provisional application No. 63/238,370, filed on Aug. 30, 2021.

(51) Int. Cl. C09B 67/20; C08J 5/18; C08K 5/18; C08K 5/3437; C08K 5/375; C08K 5/378; C08K 5/45; C09B 1/32; C09B 1/58; C09B 57/08; C09B 69/10; G02F 1/1333; G02F 1/1339; G02F 1/1343

(52) U.S. Cl. CPC C09B 67/0063; C08J 5/18; C08K 5/18; C08K 5/3437; C08K 5/375; C08K 5/378; C08K 5/45; C09B 1/32; C09B 1/585; C09B 57/08; C09B 69/109; G02F 1/133302; G02F 1/13394; G02F 1/13439; C08J 2333/14; C08J 2381/06

(57) ABSTRACT

The present disclosure relates generally to multimeric dyes, colored dielectric polymer materials, methods of making them and uses thereof. In particular, the application concerns a colored dielectric polymer material comprising a multimeric dye with structure

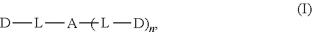

wherein each D is independently a chromophoric unit; each L is independently absent or is a linking group comprising no more than 10 atoms in length as measured in the shortest path from D to A; A is a bridging group; and n is an integer in the range of 1 to 3.

### CROSS-REFERENCE TO RELATED APPLICATIONS

This application claims the benefit of priority of U.S. Provisional Application No. 63/238,370, filed Aug. 30, 2021, and incorporated herein by reference in its entirety.

### BACKGROUND OF THE DISCLOSURE

### Field

The present disclosure relates to colored dielectric polymer materials, specifically to dielectric polymers materials having dyes therein, and films thereof, and associated devices and methods of making them.

### Technical Background

Development of the next generation of electronic devices is based on organic materials, flexible substrates and low-cost solution processing. An important material component in many organic electronic devices is a polymeric dielectric material. Such materials can serve a variety of purposes in devices, for example, as the gate insulator material in a thin-film transistor or to isolate two electrical contacts in capacitors and in display pixel elements. The polymer dielectric layer can be formed on either the gate contact (for bottom-gate transistor structures) or the semiconductor layer (for top-gate transistor structures) by depositing a solution of an electrically insulating (i.e., dielectric) polymer via solution phase process such as spin-coating or printing. To create a robust, insoluble dielectric material, a crosslinking step usually is required. Crosslinked dielectric films can be prepared, for example, by irradiation, chemical initiators, thermal treatment or combination thereof.

Liquid crystal displays of different types have different specific needs for the materials providing the coloration of the pixel. However, all require the use of colored films, e.g., red, yellow, blue, green, black, located in the proximity of layers of liquid crystal cells. In conventional LCD architectures, colored optically-clear adhesives are provided in relatively thick layers between the layers of cholesteric liquid crystal cells. These layers tend to be thick, at least 30 microns in thickness. Further, conventional synthesis protocols require harsh deposition and annealing conditions that can degrade organic dyes.

Colored dielectric films (e.g., colored red, yellow, blue, green, or black) are widely used as color films in various active-matrix pixel devices that are used to display images. These devices typically include an electronic component, the active matrix transistor, and a light source component, which could be part of the device or instead could originate from elsewhere (e.g., as ambient light). Light manipulation in each pixel for defining the image can be carried out using any of a variety of types of transmissive or reflective-type light technologies such as electrophoretic (EP), electrowetting (EW), liquid crystal (LC), and inorganic or organic light emitting diode (LED). A variety of transistor pixel control units are suitable for use, such as vapor-phase processed amorphous/polysilicon silicon transistors (a-/psSi TFTs), vapor-phase processed indium-gallium-zinc oxide (IGZO) transistors (IGZO-TFTs), or solution-processed organic transistors (OTFTs). The transistor pixel control units and the solution-processed color films are usually fabricated in separated steps, often on different substrates, due to issues with process incompatibility, such as colorfastness of colored film materials to the deposition of conducting oxide layers.

Additional improvements in colored dielectric polymer materials are necessary to enable advances in color display technologies.

### SUMMARY

The inventors have determined that dyes formed of multimeric, covalently linked chromophores can demonstrate improved stability during manufacturing processes as compared to a corresponding dye bearing a single such chromophore. In particular, high-temperature annealing has been found to detrimentally affect the color stability of many conventional dyes when provided in polymer films. When chromophores are covalently linked into a dye to form, for example, tethered dye molecule dimers, trimers, or tetramers, the resultant multimeric dyes can exhibit improved stability while maintaining excellent coloration properties. In various desirable embodiments, such multimeric dyes may be cast into thin films and be incorporated into advanced devices without experiencing significant color degradation.

Accordingly, in one aspect, the present disclosure provides a multimeric dye having the structure of Formula (I):

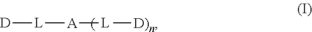

wherein
each D is independently an isolated chromophore;
each L is independently absent or is a linking group that is no more than 10 atoms in length as measured in the shortest path from A to D;
A is a bridging group; and
n is an integer in the range of 1 to 3.
   As noted above, each D is an “isolated” chromophore, i.e., the -L- and -A- moieties cannot together serve to conjugate chromophores to one another to provide an extended pi electron system.

In various embodiments of the multimeric dye as otherwise described herein, when n is 1 or 2, A is:
C₁-C₁₀ alkylene or C₂-C₁₀ alkenylene, wherein optionally 1-6 carbon atoms of the backbone of each alkylene and/or alkenylene are each independently replaced by O, N(R^{A})₀₋₁, or S;
a monocyclic or spiro and/or fused polycyclic ring system, wherein the ring system comprises cycloalkyl, heterocycloalkyl, aromatic, and/or heteroaromatic rings, and wherein the ring system comprises no more than five total rings, and wherein the ring system optionally comprises 1-3 internal bridge groups X, wherein each X is independently a bond, —C(R^{A})(R^{B})—, —O—, —N(R^{A})—, —Si(R^{A})₀₋₂—, —S(O)₀₋₂—, or —C(R^{A})(R^{B})—C(R^{A}R^{B})—; or
polysiloxane, wherein each Si is optionally substituted with 1-2 methyl groups, or 1-2 phenyl groups, and comprises no more than 20 Si atoms; and
   when n is 3, A is:
C₄-C₄₀ alkylene or C₆-C₄₀ alkenylene, wherein optionally 1-6 carbon atoms of the backbone of each alkyl and/or alkenyl are each independently replaced by O, N(R^{A})₀₋₁, or S; or
a monocyclic or spiro and/or fused polycyclic ring system, wherein the ring system comprises cycloalkyl, heterocycloalkyl, aromatic, and/or heteroaromatic rings, and wherein the ring system comprises no more than six total rings, and wherein the ring system optionally comprises 1-3 internal bridge groups X, wherein each X is independently a bond, —C(R^{A})₀₋₁(R^{B})₀₋₁—, —O—, —N(R^{A})₀₋₁—, —Si(R^{A})₀₋₂—, —S(O)₀₋₂—, or —C(R^{A})(R^{B})—C(R^{A})(R^{B})—; and
   L is absent, C₁-C₁₀ alkylene, C₂-C₁₀ alkenylene, —O—(C₁-C₈ alkylene)—O₀₋₁; —O—, —S—, —N(R^{A})—, —NHC(O)—, —C(O)O—, —C(O)—, —C(O)N(R^{A})(C₁-C₄ alkylene)—, —(C₁-C₄ alkylene)N(R^{A})C(O)—; —(C₁-C₃ alkylene)-C(O)N(R^{A})(C₁-C₃ alkylene)—;
   wherein
each alkylene, alkenylene, cycloalkyl, heterocycloalkyl, aryl, and heteroaryl is independently and substituted with 0-5 R^{A}; and
R^{A} and R^{B} are each independently selected in each occurrence from H, methyl, ethyl, propyl F, Cl, Br, oxo where chemically appropriate, —CF₃, or —OCH₃.

In various aspects, the disclosure as otherwise described herein provides for a colored dielectric polymer material comprising a dielectric polymer and the multimeric dye as otherwise described herein.

In various desirable aspects of the disclosure, the colored dielectric polymer material is in contact with a transparent conducting oxide film, such as an indium-tin oxide film.

In another aspect, the present disclosure provides for a device comprising the colored dielectric polymer material as otherwise described herein in contact with a transparent conducting oxide film.

In another aspect, the present disclosure provides for a method of making a device as otherwise described herein, comprising forming a film of the colored dielectric polymer material, depositing a transparent conducting oxide electrode thereon, and annealing at a temperature of at least 200° C. for a time of at least 10 minutes (e.g., in the range of 10 minutes to 24 hours).

Other aspects of the disclosure will be apparent to those skilled in the art in view of the description that follows.

### BRIEF DESCRIPTION OF THE DRAWINGS

FIG. 1A is a schematic cross sectional view of a liquid crystal cell according to an example embodiment.

FIG. 1B is a schematic cross sectional view of a display device according to an example embodiment.

FIG. 2 displays UV-Vis spectra of thin films (TFY-41-C and TFY-41) comprised of yellow monomeric dye (dye-59) and dimeric dye (dye-117), respectively, in polymer matrixes before and after thermal annealing according to example embodiments.

FIG. 3 displays TGA of monomeric dye (dye-59) and dimeric dye (dye-117) according to example embodiments.

FIG. 4 displays UV-Vis spectra of thin films (TFY-25-C and TFY-25) comprised of red monomeric dye (LB) and dimeric dye (dye-47), respectively, in polymer matrixes before and after thermal annealing according to example embodiments.

FIG. 5 displays TGA of monomeric dye (LB) and dimeric dye (dye-47) according to example embodiments.

FIG. 6A displays UV-Vis spectra of thin film comprised of dye-133 in polymer matrix before and after thermal annealing according to example embodiments. FIG. 6B displays TGA of dye-133 according to example embodiments. FIG. 6C displays UV-Vis spectra of thin film comprised of dye-MS165 in polymer matrix before and after thermal annealing according to example embodiments. FIG. 6D displays TGA of dye-165 according to example embodiments.

FIG. 7A displays UV-Vis spectra of a typical patterned film (Film TFY-37) comprised of dye-115 before and after AZ photolithographic process (PLP) via hole of patterned TFY-37 film with dimensions ˜5 um, according to example embodiments. FIG. 7B displays a photomicrograph of the hole-patterned film.

FIG. 8 displays UV-Vis spectra of a typical film (Film TFY-38 comprised of dye-115) before and after ITO fabrication process according to example embodiments.

FIG. 9A displays UV-Vis spectra of thin films TFY-35 comprised of Dye-51, before and after solar light exposure (SLE) according to example embodiments. FIG. 9B displays UV-Vis spectra of thin films TFY-25 comprised of Dye-47, before and after solar light exposure (SLE) according to example embodiments

FIG. 10 displays the leakage current measured from a typical thin film (TFY-37 comprised of dye-115), according to an example embodiment.

### DETAILED DESCRIPTION

The present inventors have noted that organic dyes may be advantageously utilized in the construction of devices for next generation displays. However, the present inventors have found that such dyes are often unstable to harsh process conditions associated with device manufacture. For example, the deposition of high quality indium tin oxide films requires high temperature annealing, which can lead to degradation of coloration. The present inventors have found that covalently linking chromophores together into a multimeric dye has the unexpected effect of stabilizing the dye molecules towards high temperature treatment without substantially altering coloration as compared to analogous single-chromophore monomeric dyes. Thus, the materials incorporating such multimeric dyes advantageously resist degradation throughout subsequent processing steps, such as oxide sputtering and/or annealing.

Accordingly, in one aspect, the present disclosure provides a multimeric dye having the structure of Formula (I):

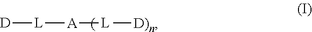

wherein
each D is independently an isolated chromophore;
each L is independently absent or is a linking group that is no more than 10 atoms in length as measured in the shortest path from A to D;
A is a bridging group; and
n is an integer in the range of 1 to 3.

As the person of ordinary skill in the art will appreciate, bridging group A has an appropriate valency for the number of -L-D groups present. For example, when n is 1, A is a divalent bridging group. When n is 2, A is trivalent. When n is 3, A is tetravalent (i.e., is connected to four -L-D groups as otherwise described herein).

A wide variety of “A” groups can be used, depending on the value of n. For example, in various embodiments of the multimeric dyes of Formula I as otherwise described herein,
n is 1 or 2, and
A is:
C₁-C₁₀ alkylene or C₂-C₁₀ alkenylene, wherein optionally 1-6 carbon atoms of the backbone of each alkylene and/or alkenylene are each independently replaced by O, N(R^{A})₀₋₁, or S;
   monocyclic or spiro and/or fused polycyclic ring system, wherein the ring system comprises cycloalkyl, heterocycloalkyl, aromatic, and/or heteroaromatic rings, and wherein the ring system comprises no more than five total rings, and wherein the ring system optionally comprises 1-3 internal bridge groups X, wherein each X is independently a bond, —C(R^{A})(R^{B})—, —O—, —N(R^{A})—, —Si(R^{A})₀₋₂—, —S(O)₀₋₂—, or —C(R^{A})(R^{B})—C(R^{A}R^{B})—; or
   polysiloxane, wherein each Si is optionally substituted with 1-2 methyl groups, or 1-2 phenyl groups, and comprises no more than 10 Si atoms,
wherein
   each alkylene, alkenylene, cycloalkyl, heterocycloalkyl, aryl, and heteroaryl is independently and substituted with 0-5 R^{A}
   R^{A} and R^{B} are each independently selected in each occurrence from H, methyl, ethyl, propyl, F, Cl, Br, oxo where chemically appropriate, —CF₃, or —OCH₃.

As another example, in various embodiments of the multimeric dyes of Formula I as otherwise described herein,
A is:
   C₄-C₄₀ alkylene or C₆-C₄₀ alkenylene, wherein optionally 1-6 carbon atoms on the backbone of each alkyl and/or alkenyl are each independently replaced by O, N(R^{A})₀₋₁, or S;
   a monocyclic or spiro and/or fused polycyclic ring system, wherein the ring system comprises cycloalkyl, heterocycloalkyl, aromatic, and/or heteroaromatic rings, and wherein the ring system comprises no more than six total rings, and wherein the ring system optionally comprises 1-3 internal bridge groups X, wherein each X is independently a bond, —C(R^{A})₀₋₁(R^{B})₀₋₁—, —O—, —N(R^{A})₀₋₁—, —S(O)₀₋₂—, or —C(R^{A})(R^{B})—C(R^{A})(R^{B})—;
wherein
   each alkylene, alkenylene, cycloalkyl, heterocycloalkyl, aryl, and heteroaryl is independently and substituted with 0-5 R^{A}
   R^{A} and R^{B} are each independently selected in each occurrence from H, methyl, ethyl, propyl, F, Cl, Br, oxo where chemically appropriate, —CF₃, or —OCH₃.

Similarly, A wide variety of “L” groups can be used. For example, in various embodiments of the multimeric dyes of Formula I as otherwise described herein,
L is absent, C₁-C₁₀ alkyl, C₂-C₁₀ alkenyl, —O—(C₁-C₈ alkyl)—O₀₋₁; —O—, —S—, —N(R^{A})—, —NHC(O)—, —C(O)O—, —C(O)—, —C(O)N(R^{A})(C₁₋C₄ alkyl)—, —(C₁-C₄ alkyl)N(R^{A})C(O)—; —(C₁-C₃ alkyl)—C(O)N(R^{A})(C₁-C₃ alkyl)—;
wherein
   each alkylene, alkenylene, cycloalkyl, heterocycloalkyl, aryl, and heteroaryl is independently and substituted with 0-5 R^{A}; and
   R^{A} and R^{B} are each independently selected in each occurrence from H, methyl, ethyl, propyl, F, Cl, Br, oxo where chemically appropriate, —CF₃, or —OCH₃.
      The multiple L groups may be the same or different from one another in any given molecule.

A wide variety of isolated chromophores D can be used. Each isolated chromophore is a radical fragment that can covalently bond to L or to A, as appropriate. As used herein, an isolated chromophore has an absorbance of at least 2,000 M⁻¹cm⁻¹ (i.e., for a molecule D-H) at a wavelength within the 380-750 nm wavelength range. In various embodiments, each isolated chromophore has an absorbance of at least 4,000 M⁻¹cm⁻¹, or even at least 6,000 M⁻¹cm⁻¹ at a wavelength within the 380-750 nm wavelength range. For example, isolated chromophore D may be derived from a known dye molecule considering the location of a hydrogen atom or an alkyl fragment (e.g., an ethyl fragment) as the site for bonding to the -L- (or the -A- when the -L- is absent). In various desirable embodiments, an isolated chromophore unit has an absorbance maximum in the range of 380-1000 nm. When the dye is not a black dye, it also has substantial transmittance at one or more other wavelengths within the 380-750 nm wavelength range. The person of ordinary skill in the art may select the suitable location for connectivity to the L or the A moiety based on chemical considerations and potential effects on the optoelectronic properties of the resulting isolated chromophore. The multiple isolated chromophores D in any given multimeric dye molecule can in some embodiments be the same as one another. In other embodiments, multiple isolated chromophores D in a multimeric dye molecule are different from one another.

The compounds can be defined generically as with respect to formula (I), or in various subgenera formulae in which A, L, and D are optionally independently selected from the groups (1a) et seq., (2a) et seq., and (3a) et seq., defined herein below (e.g., wherein the compound is of a structural formula as defined in any combination of the embodiments below):

In various embodiments of the compounds as otherwise described herein, A is independently selected from the following groups (1a)-(1bb):
(1a) n is 1, and A is C₁-C₈ alkylene, —(CH₂CH₂—O)ₚCH₂CH₂—, —(CH₂CH₂—Si(R^{A})₂)ₚCH₂CH₂—, or —(CH₂CH₂N(R^{A}))ₚCH₂CH₂—, wherein p is an integer in the range of 1-4;
(1b) n is 1, and A is unsubstituted C₁-C₆ alkyl, —(CH₂CH₂—O)ₚCH₂CH₂—, —(CH₂CH₂—Si(R^{A})₂)ₚCH₂CH₂—, or (CH₂CH₂—N(R^{A}))ₚCH₂CH₂—, wherein p is an integer in the range of 2-3;
(1c) n is 2, and A has the formula N[(C₁-C₆ alkyl)-]₃, CH[(C₁-C₆ alkyl)-]₃, or C(CH₃)[(C₁-C₆ alkyl)-]₃;
(1d) n is 1 or 2, and A is a ring system of formula Y—X—(—Y)ₙ, in which each Y is independently a monocyclic cycloalkyl, heterocycloalkyl, aromatic, or heteroaromatic ring;
(1e) A is Y—X—(—Y)ₙ, in which Y is cyclohexyl, phenyl, triazolyl, or pyridyl;
(1f) the embodiment of (1d) or (1e), wherein n is 1, and X is a bond, —CH₂—, —C(CH₃)₂—, —C(CF₃)₂—, —O—, —N(H)—, —Si(Me)₂-, —S(O)₀₋₂—, —CH₂—CH₂—, —C(CH₃)₂C(CH₃)₂—, or —C(CF₃)₂C(CF₃)₂—;
(1g) the embodiment of (1d) or (1e), wherein n is 2, and X is CH, C(CH₃), C(CF₃), or N;
(1h) n is 1, 2, or 3, A is a monocylic, fused bicyclic or fused tricyclic ring system;
(1i) the embodiment of (1h), wherein A is comprised of one or more aryl rings;
(1j) the embodiment of (1h), wherein A is cyclohexyl, phenyl, benzyl, pyridinyl, triazinyl, naphthyl, tetrahydonaphthyl, fluorenyl, or carbazolyl;
(1k) the embodiment of any of embodiments (1h)-(1j), wherein A is substituted by 1-3 R^{A}, where R^{A} is CH₃, —OCH₃, F, Cl, or —CF₃;
(1l) n is 1 or 2, and A is comprised of n+2 monocyclic rings joined by n+1 bridging groups X;
(1m) the embodiment of (1l), wherein the monocyclic rings are each independently cyclohexyl, phenyl, fluorenyl, or triazinyl (e.g., are phenyl);
(1n) the embodiment of (1l) or (1m), wherein the monocyclic rings are phenyl;
(1o) any of the embodiments (1l)-(1m), wherein n is 1, and A has the structure

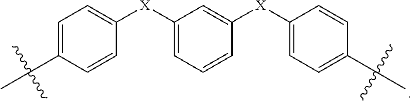

(1p) any of the embodiments (1l)-(1m), wherein n is 2, and A has the structure

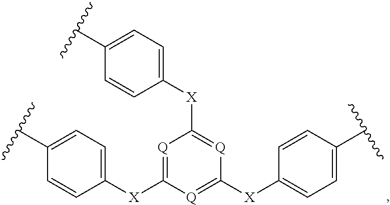

wherein each Q is independently CH or N (e.g., each Q is CH, or each Q is N);
(1q) any of the embodiments (1l)-(1p), wherein the n+1 bridging groups X are equivalent;
(1r) any of the embodiments (1l)-(1p), wherein the n+1 bridging groups X are inequivalent;
(1s) any of the embodiments (1l)-(1r), wherein each X is independently selected from the group consisting of a bond, —O—, CH₂, C(CH₃)₂, and C(CF₃)₂;
(1t) n is 1, and A comprises four monocyclic rings joined by three bridging groups X, wherein X is independently at each occurrence —O—, CH₂, C(CH₃)₂, —Si(Me)₂—, S(O)₀₋₂, or C(CF₃)₂;
(1u) the embodiment (1t), wherein the monocyclic rings are phenyl rings;
(1v) n is 1, and A comprises a polysiloxane, wherein each Si is optionally substituted with 2 methyl groups or 2 phenyl groups, and comprises no more than 6 Si atoms (e.g., comprises 2-6 Si atoms).
(1w) n is 3, and A is C—[(C₁-C₆ alkyl)-]₄ or [—(C₁-C₆ alkyl)-]₂N—(C₂-C₆ alkyl)-N—[(C₁-C₆ alkyl)-]₂.
(1x) the embodiment (1w), wherein each alkyl is linear or branched.
(1y) n is 3, and A is a ring system comprising:
   two monocyclic rings joined by one bridging group X or a moiety X—Z—X, wherein each monocyclic ring is bound to two L, if present, or, if L is absent, two D; or
   four monocyclic rings joined by one bridging group X or a moiety X—Z—X, wherein each monocyclic ring is bound to one L, if present, or, if L is absent, one D;
   wherein Z is a ring system comprising one monocyclic ring or a fused or spiro bicyclic ring.
(1z) the embodiment (1y), wherein the monocyclic rings are cyclohexyl, phenyl, pyridyl, triazinyl, dioxanyl, or triazolyl (e.g., phenyl);
(1aa) the embodiment (1y) or (1z), wherein each X is independently a bond, —O—, CH₂, C(CH₃)₂, S(O)₀₋₂, or C(CF₃)₂.
(1bb) the embodiment (1y) or (1z), wherein X is independently —O— or CH_{2.}

In various embodiments of the compounds as otherwise described herein (e.g., by any of embodiments 1a-1bb), each L is independently selected from one of the following groups (2a)-(2d):
(2a) absent, C₁-C₃ alkyl, —(C₁-C₄ alkyl)-O—, —O—, —S—, —Si(R^{A})₂—, —N(R^{A})—, —NHC(O)—, —C(O)O—, —C(O)—, or —(C₁-C₄ alkyl)N(R^{A})C(O)—;
(2b) absent, C₁-C₃ alkyl, or —O—;
(2c) —(C₁-C₄ alkyl)-O— or —(C₁-C₄ alkyl)N(R^{A})C(O)—;
(2d) absent.

Consistent with the above, the -L-A-(L-)ₙ moieties can be a wide variety of sizes, as long as they serve to isolate the chromophores (i.e., so that they cannot conjugate into a larger chromophore). For example, in various embodiments as otherwise described herein, —L-A-(L-)ₙ has at least 6 non-hydrogen atoms, e.g., at least 8 non-hydrogen atoms, or at least 10 non-hydrogen atoms. In various embodiments as otherwise descried herein, -L-A-(L-)ₙ has no more than 50 non-hydrogen atoms, e.g., no more than 40 non-hydrogen atoms.

Each of the -L- and -A- groups described above can be provided as described, i.e., not further substituted. However, in other embodiments, the -L- and -A- groups described above can be substituted. For example, in various embodiments, an -L- or -A- group as described above is substituted by one or more (for example, 1-6) substituents selected from (C₁-C₄ alkoxy), (C₁-C₄ alkyl)-C—, —Si(C₁-C₄ alkyl) and fluoro.

In various particular embodiments of multimeric dyes as otherwise described herein, the moiety -L-A-(L-)ₙ is one of the structures in Table 1, below. These structures can be further substituted as described above to provide other -L-A-(L-)ₙ moieties of the disclosure.

TABLE 1

| Structure No. | —L—A—(L—)ₙ |
|---|---|
| —L—A—(L—)ₙ 1 | 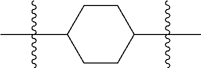 [SMILES: *C1CCC(*)CC1] |
| —L—A—(L—)ₙ 2 | 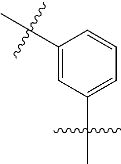 [SMILES: *c1cccc(*)c1] |
| —L—A—(L—)ₙ 3 | 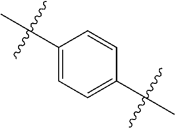 [SMILES: *c1ccc(*)cc1] |
| —L—A—(L—)ₙ 4 | 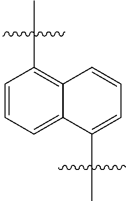 [SMILES: *c1cccc2c(*)cccc12] |
| —L—A—(L—)ₙ 5 | 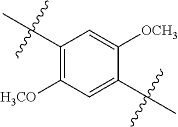 [SMILES: *c1cc(OC)c(*)cc1OC] |
| —L—A—(L—)ₙ 6 | 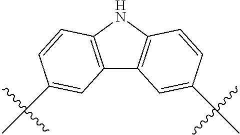 [SMILES: *c1ccc2[nH]c3ccc(*)cc3c2c1] |
| —L—A—(L—)ₙ 7 | 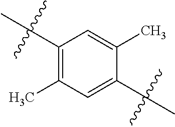 [SMILES: *c1cc(C)c(*)cc1C] |
| —L—A—(L—)ₙ 8 | 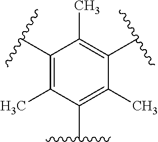 [SMILES: *c1c(C)c(*)c(C)c(*)c1C] |
| —L—A—(L—)ₙ 9 | 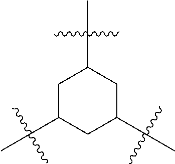 [SMILES: *C1CC(*)CC(*)C1] |
| —L—A—(L—)ₙ 10 | 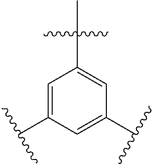 [SMILES: *c1cc(*)cc(*)c1] |
| —L—A—(L—)ₙ 11 | 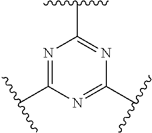 [SMILES: *c1nc(*)nc(*)n1] |
| —L—A—(L—)ₙ 12 | 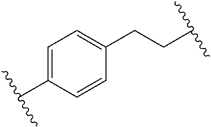 [SMILES: *CCc1ccc(*)cc1] |
| —L—A—(L—)ₙ 13 | 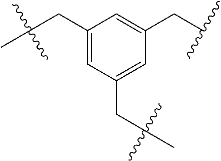 [SMILES: *Cc1cc(C*)cc(C*)c1] |
| —L—A—(L—)ₙ 14 | 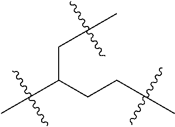 [SMILES: *CCC(*)C*] |
| —L—A—(L—)ₙ 15 | 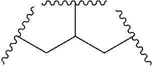 [SMILES: *CC(*)C*] |
| —L—A—(L—)ₙ 16 | 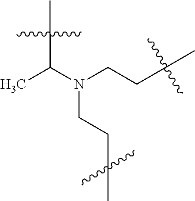 [SMILES: *CCN(CC*)C(*)C] |
| —L—A—(L—)ₙ 17 | 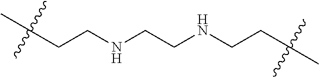 [SMILES: *CCNCCNCC*] |
| —L—A—(L—)ₙ 18 | 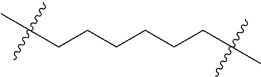 [SMILES: *CCCCCC*] |
| —L—A—(L—)ₙ 19 | 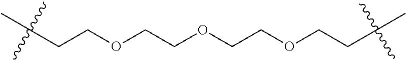 [SMILES: *CCOCCOCCOCC*] |
| —L—A—(L—)ₙ 20 | 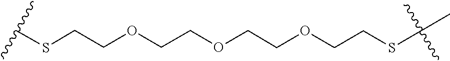 |
| —L—A—(L—)ₙ 21 | 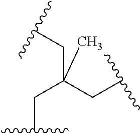 [SMILES: *CC(C)(C*)C*] |
| —L—A—(L—)ₙ 22 | 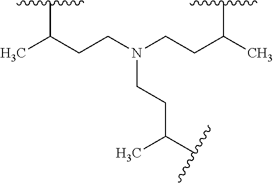 [SMILES: *C(C)CCN(CCC(*)C)CCC(*)C] |
| —L—A—(L—)ₙ 23 | 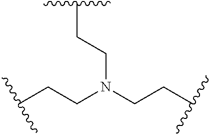 [SMILES: *CCN(CC*)CC*] |
| —L—A—(L—)ₙ 24 | 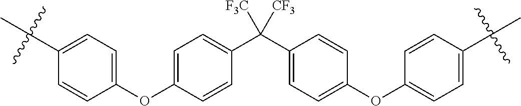 [SMILES: *c1ccc(Oc2ccc(C(c3ccc(Oc4ccc(*)cc4)cc3)(C(F)(F)F)C(F)(F)F)cc2)cc1] |
| —L—A—(L—)ₙ 25 | 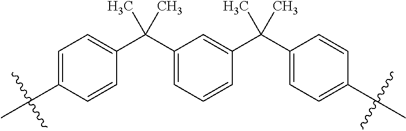 [SMILES: *c1ccc(C(C)(C)c2cccc(C(C)(C)c3ccc(*)cc3)c2)cc1] |
| —L—A—(L—)ₙ 26 | 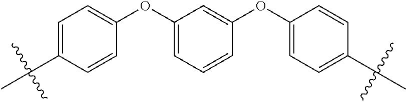 [SMILES: *c1ccc(Oc2cccc(Oc3ccc(*)cc3)c2)cc1] |
| —L—A—(L—)ₙ 27 | 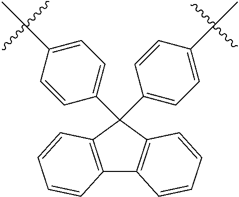 [SMILES: *c1ccc(C2(c3ccc(*)cc3)c3ccccc3-c3ccccc32)cc1] |
| —L—A—(L—)ₙ 28 | 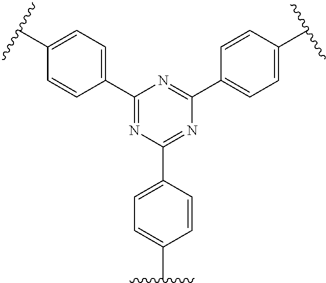 [SMILES: *c1ccc(-c2nc(-c3ccc(*)cc3)nc(-c3ccc(*)cc3)n2)cc1] |
| —L—A—(L—)ₙ 29 | 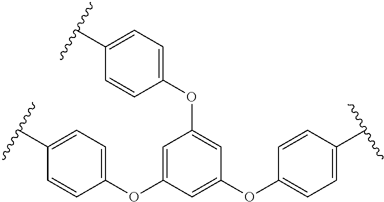 [SMILES: *c1ccc(Oc2cc(Oc3ccc(*)cc3)cc(Oc3ccc(*)cc3)c2)cc1] |
| —L—A—(L—)ₙ 30 | 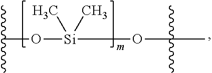 |
| | wherein m is up to 10, e.g., 2-6 |
| —L—A—(L—)ₙ 31 | 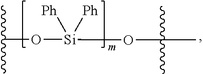 |
| | wherein m is up to 10, e.g., 2-6 |
| —L—A—(L—)ₙ 32 | 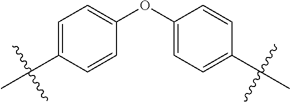 [SMILES: *c1ccc(Oc2ccc(*)cc2)cc1] |
| —L—A—(L—)ₙ 33 | 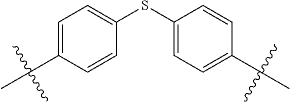 [SMILES: *c1ccc(Sc2ccc(*)cc2)cc1] |
| —L—A—(L—)ₙ 34 | 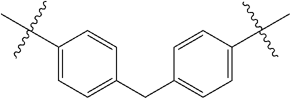 [SMILES: *c1ccc(Cc2ccc(*)cc2)cc1] |
| —L—A—(L—)ₙ 35 | 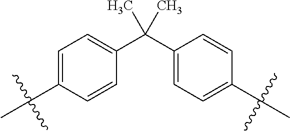 [SMILES: *c1ccc(C(C)(C)c2ccc(*)cc2)cc1] |
| —L—A—(L—)ₙ 36 | 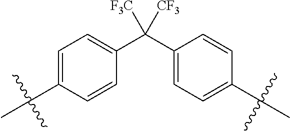 [SMILES: *c1ccc(C(c2ccc(*)cc2)(C(F)(F)F)C(F)(F)F)cc1] |
| —L—A—(L—)ₙ 37 | 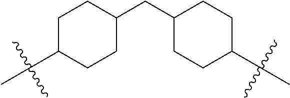 [SMILES: *C1CCC(CC2CCC(*)CC2)CC1] |
| —L—A—(L—)ₙ 38 | 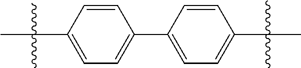 [SMILES: *c1ccc(-c2ccc(*)cc2)cc1] |
| —L—A—(L—)ₙ 39 | 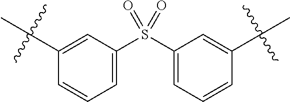 [SMILES: *c1cccc(S(=O)(=O)c2cccc(*)c2)c1] |
| —L—A—(L—)ₙ 40 | 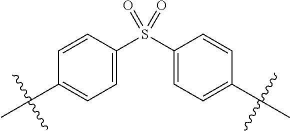 [SMILES: *c1ccc(S(=O)(=O)c2ccc(*)cc2)cc1] |
| —L—A—(L—)ₙ 41 | 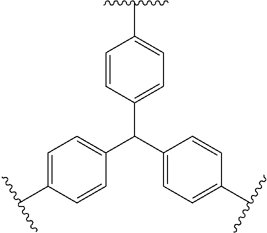 [SMILES: *c1ccc(C(c2ccc(*)cc2)c2ccc(*)cc2)cc1] |
| —L—A—(L—)ₙ 42 | 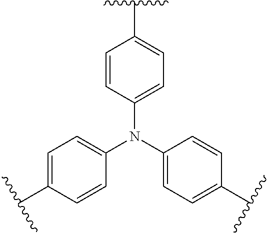 [SMILES: *c1ccc(N(c2ccc(*)cc2)c2ccc(*)cc2)cc1] |
| —L—A—(L—)ₙ 43 | 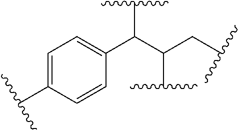 [SMILES: *CC(*)C(*)c1ccc(*)cc1] |
| —L—A—(L—)ₙ 44 | 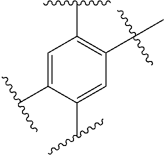 [SMILES: *c1cc(*)c(*)cc1*] |
| —L—A—(L—)ₙ 45 | 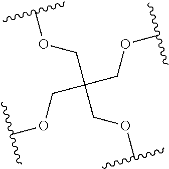 |
| —L—A—(L—)ₙ 46 | 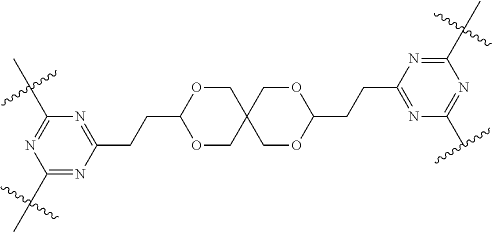 [SMILES: *c1nc(*)nc(CCC2OCC3(CO2)COC(CCc2nc(*)nc(*)n2)OC3)n1] |
| —L—A—(L—)ₙ 47 | 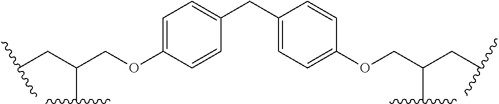 [SMILES: *CC(*)COc1ccc(Cc2ccc(OCC(*)C*)cc2)cc1] |
| —L—A—(L—)ₙ 48 | 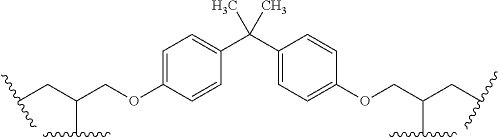 [SMILES: *CC(*)COc1ccc(C(C)(C)c2ccc(OCC(*)C*)cc2)cc1] |
| —L—A—(L—)ₙ 49 | 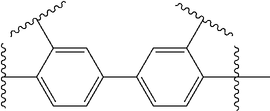 [SMILES: *c1ccc(-c2ccc(*)c(*)c2)cc1*] |
| —L—A—(L—)ₙ 50 | 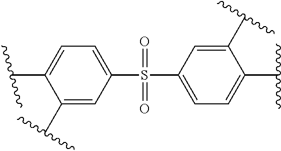 [SMILES: *c1ccc(S(=O)(=O)c2ccc(*)c(*)c2)cc1*] |
| —L—A—(L—)ₙ 51 | 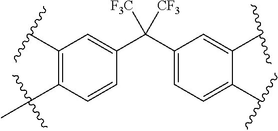 [SMILES: *c1ccc(C(c2ccc(*)c(*)c2)(C(F)(F)F)C(F)(F)F)cc1*] |
| —L—A—(L—)ₙ 52 | 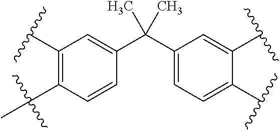 [SMILES: *c1ccc(C(C)(C)c2ccc(*)c(*)c2)cc1*] |
| —L—A—(L—)ₙ 53 | 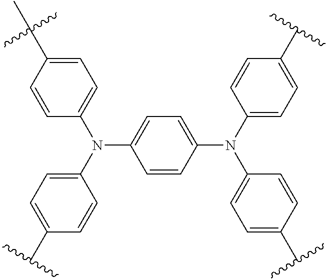 [SMILES: *c1ccc(N(c2ccc(*)cc2)c2ccc(N(c3ccc(*)cc3)c3ccc(*)cc3)cc2)cc1] |
| —L—A—(L—)ₙ 54 | 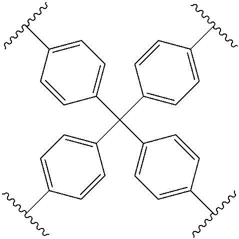 [SMILES: *c1ccc(C(c2ccc(*)cc2)(c2ccc(*)cc2)c2ccc(*)cc2)cc1] |
| —L—A—(L—)ₙ 55 | 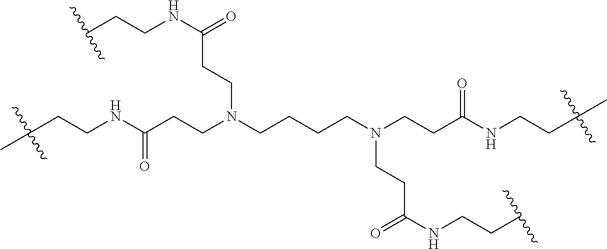 [SMILES: *CCNC(=O)CCN(CCCCN(CCC(=O)NCC*)CCC(=O)NCC*)CCC(=O)NCC*] |
| —L—A—(L—)ₙ 56 | 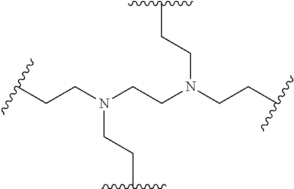 [SMILES: *CCN(CC*)CCN(CC*)CC*] |
| —L—A—(L—)ₙ 57 | 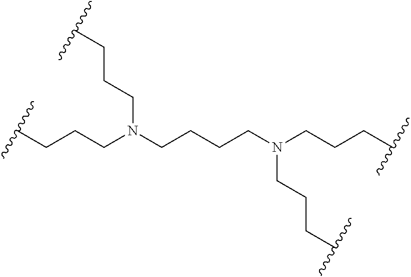 [SMILES: *CCCN(CCC*)CCCCN(CCC*)CCC*] |
| —L—A—(L—)ₙ 58 | 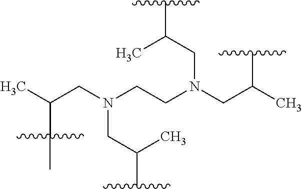 [SMILES: *C(C)CN(CCN(CC(*)C)CC(*)C)CC(*)C] |

In certain embodiments of the compounds as otherwise described herein, dye unit D is derived from one of the following dyes (3a)-(3d):
(3a) a perylene diimide chromophore, a naphthalene chromophore, a naphthalene monoimide chromophore, a perylene chromophore, an anthraquinone chromophore, a quinone chromophore, a phenazine chromophore, an azo dye chromophore, a triarylmethane chromophore, a transition metal (e.g., chromium) coordination complex chromophore, a cyanine chromophore (e.g., a phthalocyanine chromophore), a phenoxazine chromophore, an indole chromophore, a xanthene chromophore, a coumarin chromophore, a nitro dye chromophore, or an indene chromophore.
(3b) a perylene diimide chromophore, a naphthalene chromophore, a naphthalene monoimide chromophore, a perylene chromophore, an anthraquinone chromophore, a quinone chromophore, a phenazine chromophore, or an azo dye chromophore.
(3c) a perylene diimide chromophore or an anthraquinone chromophore.

In various embodiments, each isolated chromophore D has a transmittance maximum in the range of 380-450 nm (violet); 450-495 nm (blue); 495-570 nm (green); 570-590 nm (yellow); 590-620 nm (orange); or 620-750 nm (red).

Particular examples of suitable dyes which isolated chromophores D may be derived from include those of Table 2, below:

TABLE 2

| Common | |
|---|---|
| Name | Nominal Structure |
| Disperse red 60 | 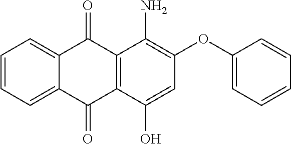 [SMILES: Nc1c(Oc2ccccc2)cc(O)c2c1C(=O)c1ccccc1C2=O] |
| Sudan Red 7B | 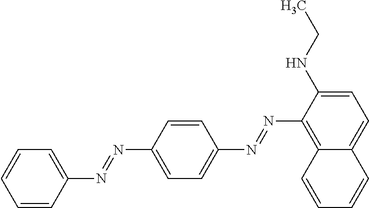 [SMILES: CCNc1ccc2ccccc2c1/N=N/c1ccc(/N=N/c2ccccc2)cc1] |
| Disperse red 11 | 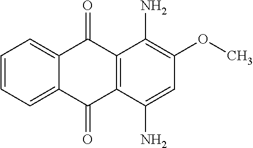 [SMILES: COc1cc(N)c2c(c1N)C(=O)c1ccccc1C2=O] |
| Sudan 3 | 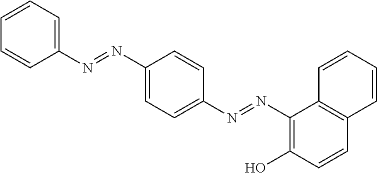 [SMILES: Oc1ccc2ccccc2c1/N=N/c1ccc(/N=N/c2ccccc2)cc1] |
| Disperse red 1 | 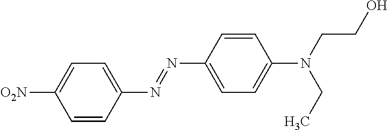 [SMILES: CCN(CCO)c1ccc(/N=N/c2ccc([N+](=O)[O-])cc2)cc1] |
| Sudan 1 | 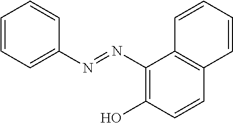 [SMILES: Oc1ccc2ccccc2c1/N=N/c1ccccc1] |
| Disperse red 72 | 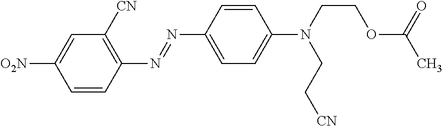 [SMILES: CC(=O)OCCN(CCC#N)c1ccc(/N=N/c2ccc([N+](=O)[O-])cc2C#N)cc1] |
| Sudan red G | 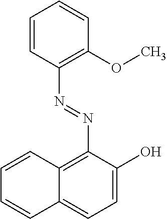 [SMILES: COc1ccccc1/N=N/c1c(O)ccc2ccccc12] |
| Disperse red 9 | 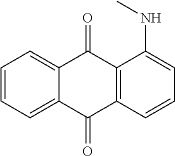 [SMILES: CNc1cccc2c1C(=O)c1ccccc1C2=O] |
| Mordant Red 11 | 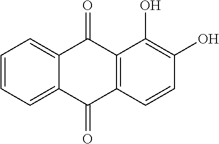 [SMILES: O=C1c2ccccc2C(=O)c2c1ccc(O)c2O] |
| Acid Red 27 | 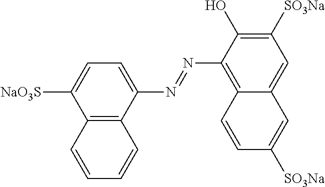 |
| Basic Red 9 | 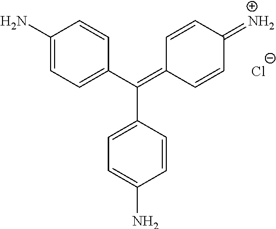 [SMILES: Nc1ccc(C(=C2C=CC(=[NH2+])C=C2)c2ccc(N)cc2)cc1 ; [Cl-]] |
| Acid Red 18 | 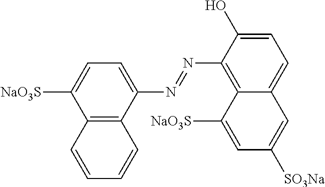 |
| Basic Red 14 | 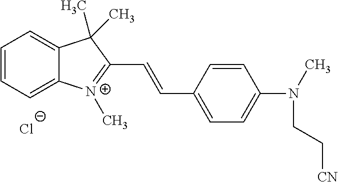 [SMILES: CN(CCC#N)c1ccc(/C=C/C2=[N+](C)c3ccccc3C2(C)C)cc1 ; [Cl-]] |
| Acid red 266 | 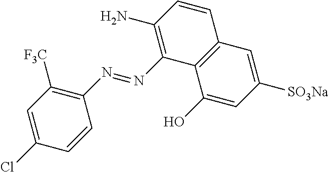 |
| Solvent red 26 | 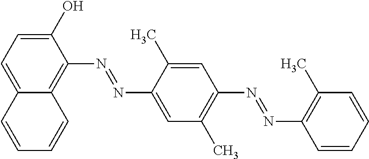 [SMILES: Cc1ccccc1/N=N/c1cc(C)c(/N=N/c2c(O)ccc3ccccc23)cc1C] |
| Nile red | 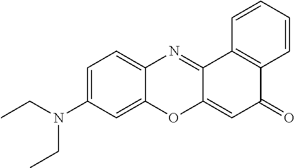 [SMILES: CCN(CC)c1ccc2nc3c4ccccc4c(=O)cc-3oc2c1] |
| Vat red 10 | 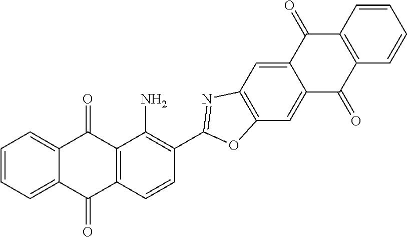 [SMILES: Nc1c(-c2nc3cc4c(cc3o2)C(=O)c2ccccc2C4=O)ccc2c1C(=O)c1ccccc1C2=O] |
| Toludine red | 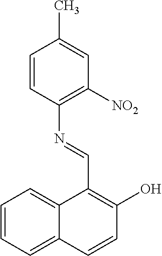 [SMILES: Cc1ccc(/N=C/c2c(O)ccc3ccccc23)c([N+](=O)[O-])c1] |
| Vat red 13 | 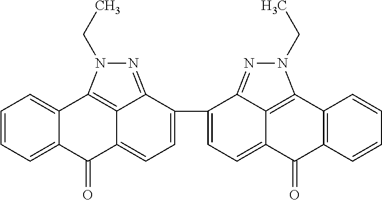 [SMILES: CCn1nc2c(-c3ccc4c5c(n(CC)nc35)-c3ccccc3C4=O)ccc3c2c1-c1ccccc1C3=O] |
| Direct red 80 | 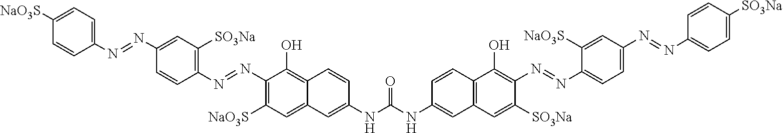 |
| Tartrazine | 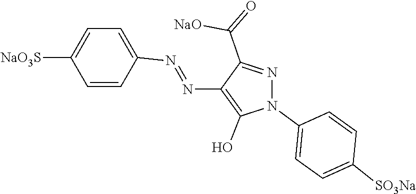 |
| Direct yellow 4 | 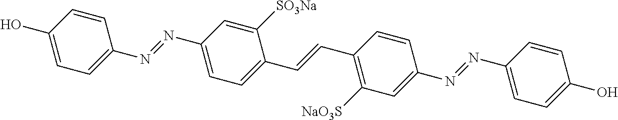 |
| Reactive yellow 4 | 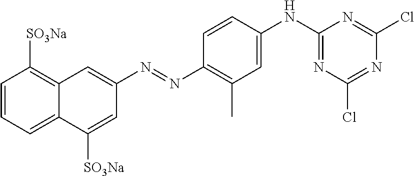 |
| Direct yellow 27 | 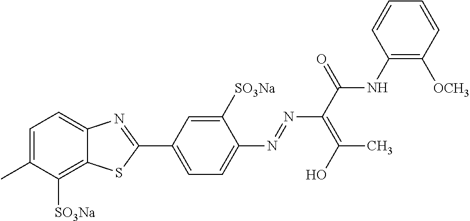 |
| Vat yellow 4 | 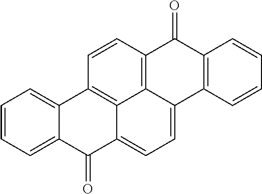 [SMILES: O=C1c2ccccc2-c2ccc3c4c(ccc1c24)-c1ccccc1C3=O] |
| Solvent yellow 94 | 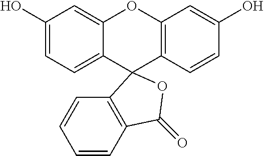 [SMILES: O=C1OC2(c3ccc(O)cc3Oc3cc(O)ccc32)c2ccccc21] |
| Vat yellow 1 | 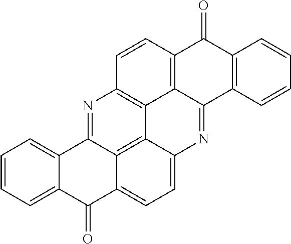 [SMILES: O=c1c2ccccc2c2nc3ccc4c(=O)c5ccccc5c5nc6ccc1c2c6c3c45] |
| Solvent yellow 44 | 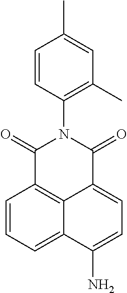 [SMILES: Cc1ccc(N2C(=O)c3cccc4c(N)ccc(c34)C2=O)c(C)c1] |
| C.I. Vat yellow 5 | 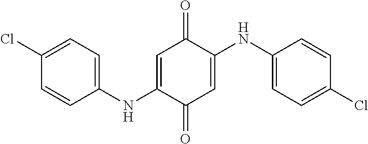 [SMILES: O=C1C=C(Nc2ccc(Cl)cc2)C(=O)C=C1Nc1ccc(Cl)cc1] |
| Solvent yellow 43 | 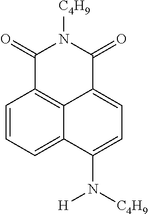 |
| Solvent yellow 33 | 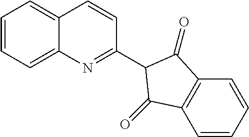 [SMILES: O=C1c2ccccc2C(=O)C1c1ccc2ccccc2n1] |
| Disperse yellow 42 | 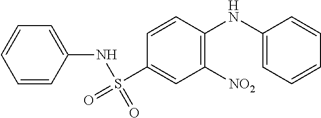 [SMILES: O=[N+]([O-])c1cc(S(=O)(=O)Nc2ccccc2)ccc1Nc1ccccc1] |
| Solvent yellow 124 | 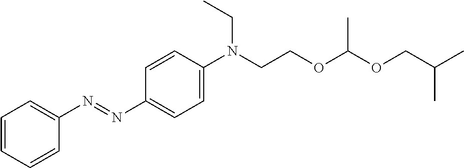 [SMILES: CCN(CCOC(C)OCC(C)C)c1ccc(/N=N/c2ccccc2)cc1] |
| Disperse yellow 31 | 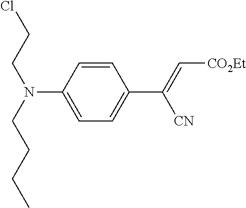 [SMILES: CCCCN(CCCl)c1ccc(/C(C#N)=C/C(=O)OCC)cc1] |
| Disperse yellow 26 | 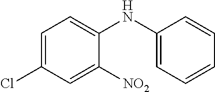 [SMILES: O=[N+]([O-])c1cc(Cl)ccc1Nc1ccccc1] |
| C.I. Basic Yellow 28 | 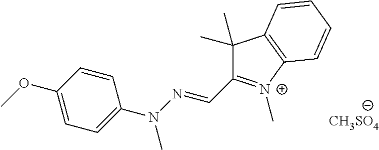 |
| Tartrazine | 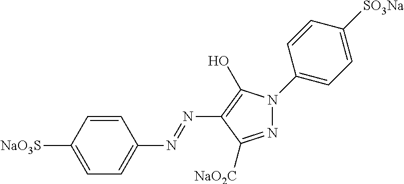 |
| Coumarin 153 | 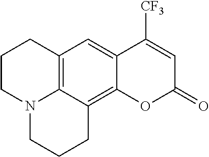 [SMILES: O=c1cc(C(F)(F)F)c2cc3c4c(c2o1)CCCN4CCC3] |
| Coumarin 30 | 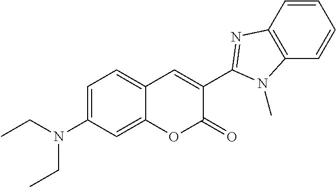 [SMILES: CCN(CC)c1ccc2cc(-c3nc4ccccc4n3C)c(=O)oc2c1] |
| Coumarin 102 | 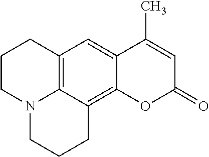 [SMILES: Cc1cc(=O)oc2c3c4c(cc12)CCCN4CCC3] |
| Coumarin 6 | 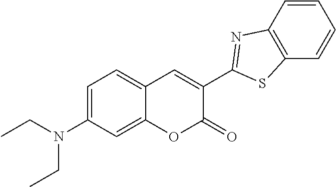 [SMILES: CCN(CC)c1ccc2cc(-c3nc4ccccc4s3)c(=O)oc2c1] |
| Malachite Green | 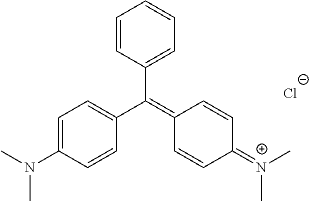 |
| Acid green 25 | 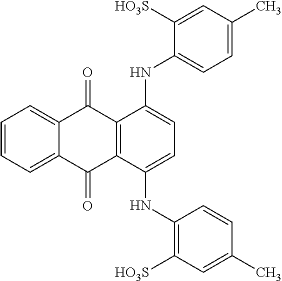 [SMILES: Cc1ccc(Nc2ccc(Nc3ccc(C)cc3S(=O)(=O)O)c3c2C(=O)c2ccccc2C3=O)c(S(=O)(=O)O)c1] |
| Solvent green 3 | 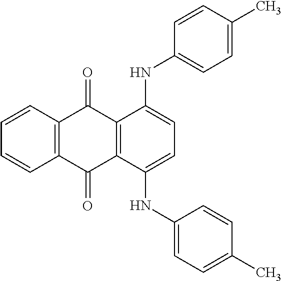 [SMILES: Cc1ccc(Nc2ccc(Nc3ccc(C)cc3)c3c2C(=O)c2ccccc2C3=O)cc1] |
| Naphthol Green B | 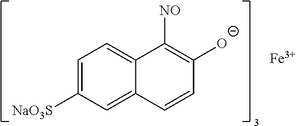 |
| Solvent green 28 | 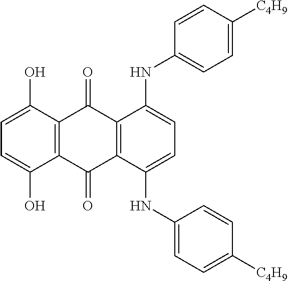 |
| Solvent green 5 | 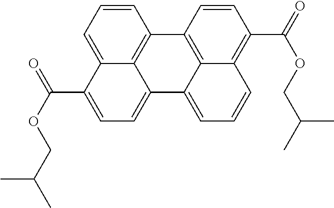 [SMILES: CC(C)COC(=O)c1ccc2c3cccc4c(C(=O)OCC(C)C)ccc(c5cccc1c52)c43] |
| Vat green 12 | 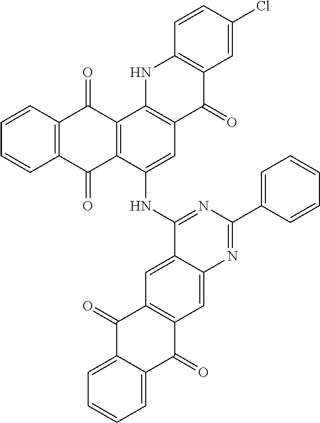 [SMILES: O=C1c2ccccc2C(=O)c2cc3c(Nc4cc5c(=O)c6cc(Cl)ccc6[nH]c5c5c4C(=O)c4ccccc4C5=O)nc(-c4ccccc4)nc3cc21] |
| Cardiogreen | 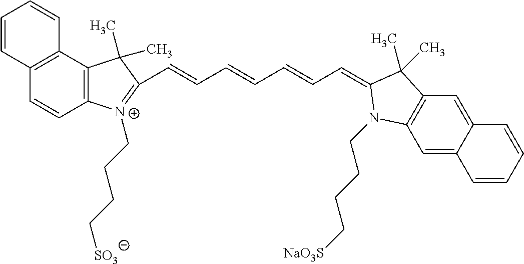 |
| Vat green 1 | 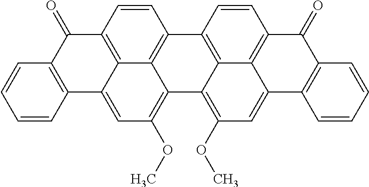 [SMILES: COc1cc2c3c(ccc4c5ccc6c7c(cc(OC)c(c1c34)c75)-c1ccccc1C6=O)C(=O)c1ccccc1-2] |
| Vat green 3 | 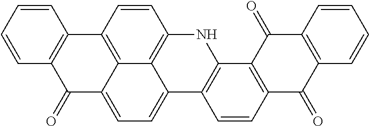 [SMILES: O=C1c2ccccc2C(=O)c2c1ccc1c2[nH]c2ccc3c4ccccc4c(=O)c4ccc1c2c43] |
| Vat green 9 | 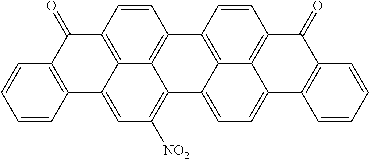 [SMILES: O=C1c2ccccc2-c2ccc3c4c([N+](=O)[O-])cc5c6c(ccc(c7ccc1c2c73)c64)C(=O)c1ccccc1-5] |
| Direct green 26 | 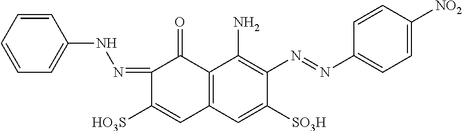 [SMILES: Nc1c(/N=N/c2ccc([N+](=O)[O-])cc2)c(S(=O)(=O)O)cc2c1C(=O)/C(=N\Nc1ccccc1)C(S(=O)(=O)O)=C2] |
| Acid green 50 | 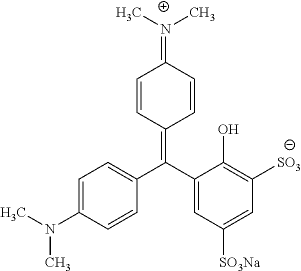 |
| Disperse green 9 | 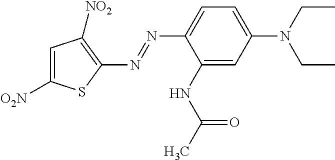 [SMILES: CCN(CC)c1ccc(/N=N/c2sc([N+](=O)[O-])cc2[N+](=O)[O-])c(NC(C)=O)c1] |
| Direct green 28 | 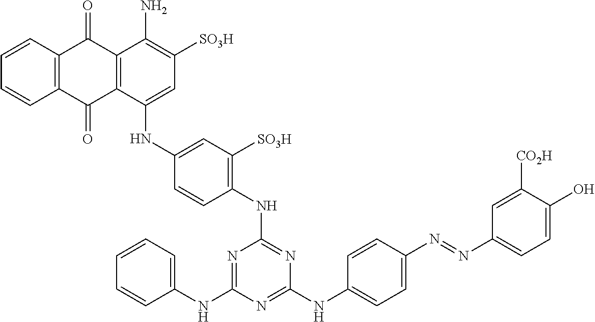 [SMILES: Nc1c(S(=O)(=O)O)cc(Nc2ccc(Nc3nc(Nc4ccccc4)nc(Nc4ccc(/N=N/c5ccc(O)c(C(=O)O)c5)cc4)n3)c(S(=O)(=O)O)c2)c2c1C(=O)c1ccccc1C2=O] |
| Vat green 11 | 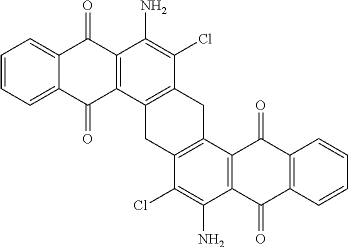 [SMILES: Nc1c(Cl)c2c(c3c1C(=O)c1ccccc1C3=O)Cc1c(Cl)c(N)c3c(c1C2)C(=O)c1ccccc1C3=O] |
| Food black 1 | 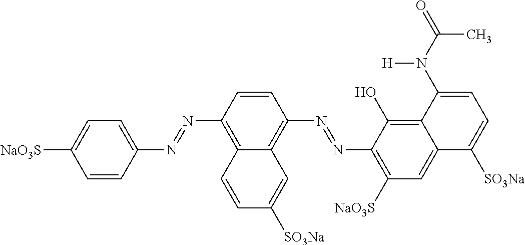 |
| Mordant Black 25 | 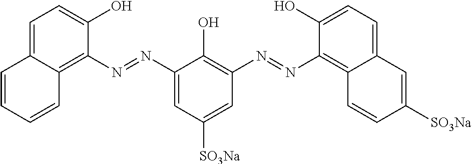 |
| Mordant Black 11 | 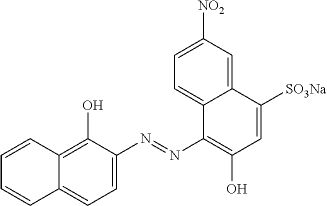 |
| Acid Black 48 | 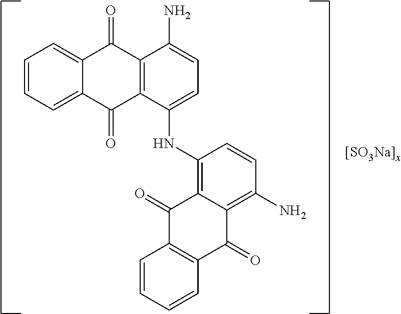 |
| Reactive Black 5 | 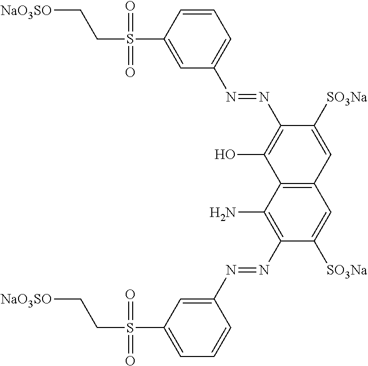 |
| Reactive Black 8 | 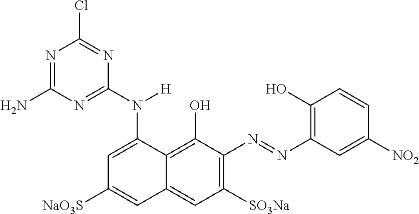 |
| -Vat Black 29 | 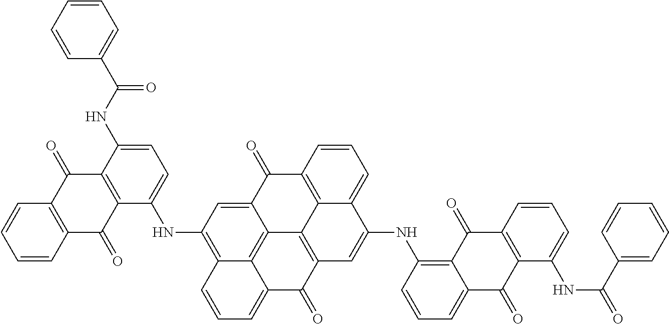 [SMILES: O=C(Nc1cccc2c1C(=O)c1cccc(Nc3cc4c5c6c3cccc6c(=O)c3cc(Nc6ccc(NC(=O)c7ccccc7)c7c6C(=O)c6ccccc6C7=O)c6cccc(c4=O)c6c3-5)c1C2=O)c1ccccc1] |
| Direct Black 19 | 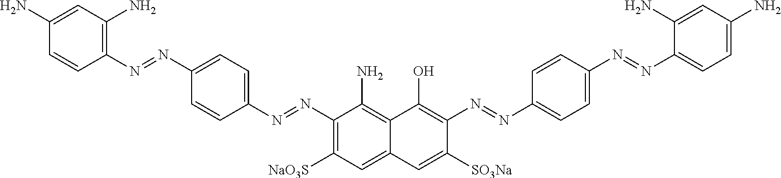 |
| Solvent Black 3 | 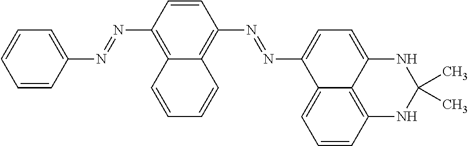 [SMILES: CC1(C)Nc2cccc3c(/N=N/c4ccc(/N=N/c5ccccc5)c5ccccc45)ccc(c23)N1] |
| Vat Black 27 | 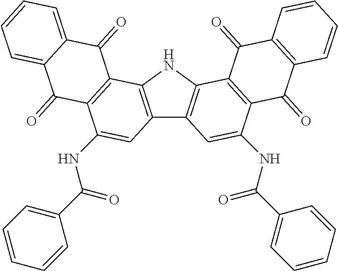 [SMILES: O=C(Nc1cc2c3cc(NC(=O)c4ccccc4)c4c(=O)c5ccccc5c(=O)c4c3[nH]c2c2c(=O)c3ccccc3c(=O)c12)c1ccccc1] |
| Vat Black 25 | 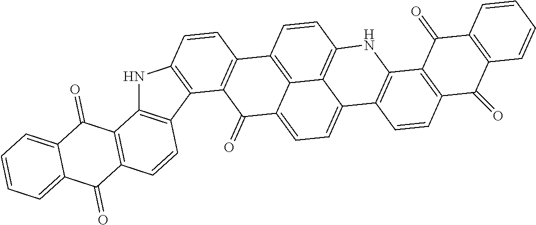 [SMILES: O=C1c2ccccc2C(=O)c2c1ccc1c2[nH]c2ccc3c4ccc5[nH]c6c7c(ccc6c5c4c(=O)c4ccc1c2c43)C(=O)c1ccccc1C7=O] |
| Direct Black 36 | |
| Solvent Black 27 | 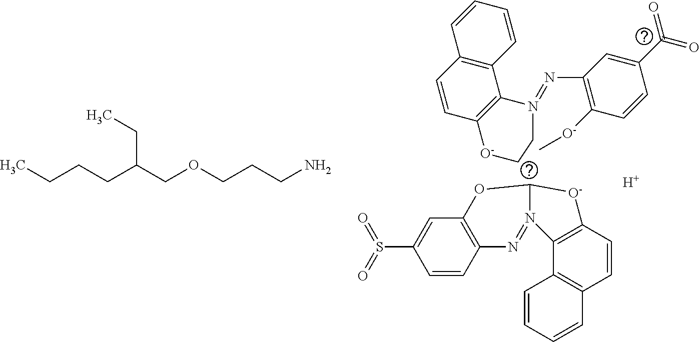 |
| Disperse | |
| Black | |
| 4 | |
| Disperse | |
| Black | |
| 9 | |
| Acid | |
| Black | |
| 1 | |
| Direct | |
| Black | |
| 195 | |
| Acid | |
| Black | |
| 180 | |
| Mordant | |
| black | |
| 9 | |
| Reactive | |
| Black | |
| 31 | |
| Azo | |

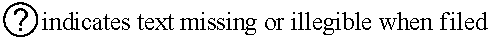

Of course, the person of ordinary skill will appreciate that a wide variety of chromophores can be used.

Each isolated chromophore D may be covalently bound to L, if present, or A at any chemically suitable site. In various embodiments, D is bound at substantially the same place in each occurrence. In other embodiments, D is bound to L, if present, or A at a mixture of locations. The attachment location or locations may be adjusted according to the selected synthetic methods for dye preparation, and/or the effect of L and/or A on the optical properties of D, with the understanding that the chromophores D are not conjugated to one another through the -L-A-(L-)ₙ moiety.

In particular embodiments as otherwise described herein, isolated chromophore D is equivalent in each occurrence. In other embodiments, the isolated chromophores D are equivalent or positional isomers. In other embodiments, different isolated chromophores D can be selected.

The multimeric dyes described herein can be provided with a variety of absorptivities in the visible range of the spectrum. For example, in certain embodiments, the multimeric dye has a has a molar absorptivity of at least 4000 M⁻¹cm⁻¹, e.g., at least 8000 M⁻¹cm⁻¹ or at least 15000 M⁻¹cm⁻¹ at a wavelength within the 380-750 nm wavelength range.

Advantageously, in an aspect of the present disclosure, the multimeric dye as otherwise described herein can be combined with a polymer to form a colored dielectric polymer material. In particular, in certain embodiments, the colored dielectric polymer material comprises a dielectric polymer, and a multimeric dye as otherwise described herein dispersed in the dielectric polymer. The colored dielectric polymer material can advantageously be provided in the form of a film.

In general, dielectric polymers are well known in the art and may be selected according to their electrical properties, processing properties (such as ability to form a thin film), and compatibility with the multimeric dyes as otherwise described herein.

A wide variety of dielectric polymers can be used. In various embodiments, suitable polymers include polyolefins (e.g., polystyrene, polyethylene, poly-alpha-methyl styrene, polyisobutene, polypropylene, polyvinyl chloride), polyesters (polymethylmethacrylate), poly (carbonates), polyimides, polynorbornene, polysulfone, and poly(p-phenyleneoxide). However, the person of ordinary skill in the art will appreciate that a wide variety of polymers can be used. As the compounds described herein are especially stable to sublimation during processing, in various embodiments the dielectric polymer is not substantially crosslinked.

However, in other the dielectric polymer is substantially crosslinked. Other suitable dielectric polymers can be prepared and processed as disclosed in International Patent Application Publication no. 2013/119717 and/or International Patent Application Publication no. 2010/057984, each of which is incorporated by reference herein in its entirety.

A variety of suitable dielectric polymers are described in the following: U.S. Pat. No. 10,551,745 B2 (polymers with cinnamoyl, extended cinnamoyl, coumarin, epoxide and bismaleimide, cols. 7-40); U.S. Pat. No. 9,929,345 B1 (dicyclopentadiene polymer curable at the backbone, cols. 9-20); U.S. Pat. No. 10,147,895 B2 (dicyclopentadiene polymer curable at the pendant groups, cols. 8-21); U.S. Pat. No. 9,923,158 B2 (coumarin as a pendant group for crosslinking, cols. 12-31); U.S. Pat. No. 9,704,997 B2 (polyoxadiazoles, cols. 9-49); U.S. Pat. No. 8,878,169 B2 (extended cinnamoyl-pendant polymers, cols. 9-40); U.S. Pat. No. 9,035,287 B2 (polysulfones, cols. 10-59); U.S. Pat. No. 7,981,989 B2 (free radical curable vinyls and acrylates, cols. 5-21); International Patent Application Publication no. 2016100983 A1 (fluorinated polymer, pages 6-16); U.S. Patent Application Publication no. 2011/0284849 (organic/inorganic hybrid, throughout), each of which is hereby incorporated herein by reference it its entirety.

In various embodiments as otherwise described herein, the polymer comprises at least one polymer comprising a repeating unit selected from:

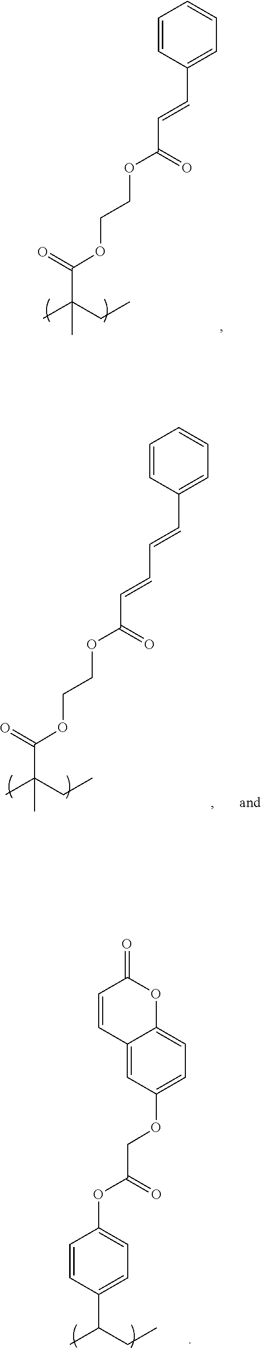

In various embodiments, the multimeric dye is dispersed within the dielectric polymer as a substantially homogenous distribution. The amount of multimeric dye may be selected according to the desired optical transmission and absorption characteristics of the colored dielectric polymer material, relative to the characteristics of the dye. In various embodiments, the colored dielectric polymer material may include more than one multimeric dye, and/or include a multimeric dye in admixture with a conventional dye. For example, in various embodiments as otherwise described herein, the dye is present in the colored dielectric polymer material in an amount of at least 1 wt %, e.g., at least 3 wt %, at least 10 wt %, or at least 50wt %. For example, in various embodiments as otherwise described herein, the dye is present in the polymer in an amount in the range of 1-80 wt % e.g., 1-80 wt %, or 1-50 wt %, or 1-20 wt %, or 3-80 wt %, or 3-50 wt %, or 3-20 wt %, or 3-10 wt %, or 5-80 wt %, or 5-50 wt %, or 5-30 wt %, or 5-20 wt %, or 10-80 wt %, or 10-50 wt %, or 20-80 wt %, or 20-50 wt %.

In various embodiments (e.g., embodiments that present a non-black color), the colored dielectric polymeric material as otherwise described herein advantageously allows a certain amount of light to pass through the material in a desired visible wavelength range. Accordingly, in various embodiments, the colored dielectric polymer material is provided as a body (e.g., a film) having a transmittance maximum intensity of at least 50% (e.g., at least 75%, at least 90%) at one or more wavelengths in the range of 380-450 nm (violet); 450-495 nm (blue); 495-570 nm (green); 570-590 nm (yellow); 590-620 nm (orange); or 620-750 nm (red). And in various such embodiments, the body of colored dielectric polymer material has a transmittance minimum intensity of no more than 20% (e.g., no more than 10%, no more than 5%) at one or more wavelengths in the range of 380-450 nm (violet); 450-495 nm (blue); 495-570 nm (green); 570-590 nm (yellow); 590-620 nm (orange); or 620-750 nm (red). In various such embodiments, the body is no more than 1 mm in thickness, e.g., no more than 100 microns, no more than 50 microns, or even no more than 10 microns in thickness. For example, in various such embodiments, the body has a thickness in the range of 0.05-100 microns, e.g., 0.05-50 microns, or 0.05-10 microns, or 0.05-5 microns, or 0.05-2 microns, or 0.05-1 micron, or 0.1-100 microns, or 0.1-50 microns, or 0.1-10 microns, or 0.1-5 microns, or 0.1-2 microns, or 0.1-1 micron, or 0.2-100 microns, or 0.2-50 microns, or 0.2-10 microns, or 0.2-5 microns, or 0.2-2 microns, or 0.2-1 micron. As the person of ordinary skill in the art, high transmittance in one part of the visible spectrum and low transmittance in another part of the visible spectrum will provide a perceived non-black color to the material.

In other embodiments, it may be advantageous for only small amounts of visible light to transmit through the colored dielectric polymer materials of the disclosure. For example, when the colored dielectric polymer material is black. Accordingly, in certain embodiments as otherwise described herein, the material is in the form of a body (e.g., a film) having a total transmittance of light in wavelength range 380-750 nm of no more than 20%, for example, no more than 10%, no more than 5%, or even no more than 1%. In various such embodiments, the body is no more than 1 mm in thickness, e.g., no more than 100 microns, no more than 50 microns, or even no more than 10 microns in thickness. For example, in various such embodiments, the body has a thickness in the range of 0.05-100 microns, e.g., 0.05-50 microns, or 0.05-10 microns, or 0.05-5 microns, or 0.05-2 microns, or 0.05-1 micron, or 0.1-100 microns, or 0.1-50 microns, or 0.1-10 microns, or 0.1-5 microns, or 0.1-2 microns, or 0.1-1 micron, or 0.2-100 microns, or 0.2-50 microns, or 0.2-10 microns, or 0.2-5 microns, or 0.2-2 microns, or 0.2-1 micron.

In particular embodiments, the colored dielectric polymer materials may be fabricated as thin films for use in devices. Accordingly, for adequate coloration in a thin film, the selected dye may be chosen to strongly absorb particular wavelengths. In various embodiments as otherwise described herein, the dye has a peak molar absorptivity of at least 4,000 M⁻¹cm⁻¹, e.g., at least 8,000 M⁻¹cm⁻¹ or at least 15,000 M⁻¹ cm⁻¹.

Polymers comprising pigments are well known in the art. As apparent to the person of ordinary skill in the art, pigments are provided as insoluble substances or particles. In various embodiments as described herein, the colored dielectric polymer material does not comprise a pigment.

Additionally, in various desirable embodiments the colored dielectric polymer material maintains desirable dielectric properties. One measure of a dielectric quality is the breakdown voltage (i.e., at a given electric field). Accordingly, in certain embodiments as otherwise described herein, the colored dielectric polymer material has a breakdown voltage of at least 50 V at 2 MV/cm (e.g., at least 60 V, or 70 V, or 80 V, or 90 V, or 100 V). Leakage current is another measure of dielectric quality; in various embodiments as otherwise described herein, the colored dielectric polymer material (e.g., in the form of a film having a thickness as described herein) has a leakage current density of no more than 1×10⁻⁸ A/cm² at an electric field of 1.0 MV/cm.

Another desired property of a colored dielectric polymer material is a relatively low dielectric constant to reduce capacitive coupling between the electrodes in the device. Accordingly, in various embodiments as otherwise described herein, the colored dielectric polymer material has dielectric constant of no more than 8, e.g., no more than 7, or no more than 6, or no more than 5, or no more than 4, or no more than 3, at 1 MHz. For example, in various embodiments, the colored dielectric polymer material has a dielectric constant in the range of 2 to 8, e.g., in the range of 2 to 7, or 2 to 6, or 2 to 5, or 2 to 4, or 2.5 to 8, or 2.5 to 7, or 2.5 to 6, or 2.5 to 5, or 3 to 8, or 3 to 7, or 3 to 6, or 3 to 5, at 1 MHz. In particular embodiments, the colored dielectric polymer material has a dielectric constant in the range of 2-6, e.g., 2-5, or 2-4, or 2-3 at 1 MHz.

Conventional techniques can be used to form films of the colored dielectric polymer materials of the disclosure. The dye can be dissolved in a solution of the polymer, which can then be cast into a film or other body. The film may be prepared through spin-coating, slit-coating, slot-die, or blade coating, among other techniques such as gravure, flexographic, or ink jet printing. Conventional negative or positive photolithographic techniques can be used to provide patterned radiation to crosslink the base polymers to provide the crosslinked material. Radiation, heat, or combination of both can be used to crosslink the film which then can be patterned using conventional photoresists. Synthetic processes for polymer materials can be found in the references cited.

Subsequent to film formation, the colored dielectric polymer material of the present teachings can be subject to further patterning and process steps, by which additional layers, including additional dielectric, semiconductor and/or conducting layers, can be formed on top of the dielectric material.

The transparent conduct oxide electrode as otherwise described herein may be prepared according to methods known to the person of ordinary skill in the art. For example, in certain embodiments, the transparent conducting oxide electrode is deposited on the colored dielectric polymer material film through spluttering followed by annealing.

Advantageously, in various embodiments the colored dielectric polymer material as otherwise described herein resists degradation due to the sputtering and/or annealing steps during deposition of a transparent conducting oxide. Accordingly, in certain embodiments as otherwise described herein, the intensity of the maximum transmittance of the colored dielectric polymer material after sputtering and annealing is within 20% of the intensity of the maximum transmittance prior to sputtering and annealing, and/or the intensity of the maximum absorbance in an absorbing region in the visible spectrum is within 20% of the intensity of the maximum absorbance prior to sputtering and annealing. In addition, the breakdown voltage and leakage current of the colored dielectric polymer material film is not significantly affected by sputtering and annealing of conductive oxide films. For example, in certain embodiments, the breakdown voltage is within 20% of the breakdown voltage prior to sputtering and annealing. In certain embodiments as otherwise described herein, the leakage current of the colored dielectric polymer material is within 20% of the leakage current prior to sputtering and annealing.

In various desirable aspects the colored dielectric polymer material of the disclosure is in contact with a transparent conducting oxide film, such as an indium-tin oxide film. As noted above and described below, the present inventors have determined that the colored dielectric polymer materials described herein can be especially stable, even under the rigorous conditions used to process transparent conducting oxides, and under other conditions used in patterning and processing of thin-film devices.

Thus, in another aspect, the present disclosure provides for a device incorporating a colored dielectric polymeric material as described herein. In various such embodiments as otherwise described herein, the device comprises the colored dielectric polymer material in contact with a transparent conducting oxide.

Transparent conducting oxides are generally known in the art. For example, the transparent conducting oxide may be indium tin oxide (ITO), zinc tin oxide (ZTO), cadmium tin oxide, (CTO), or fluoride-doped tin oxide (FTO).

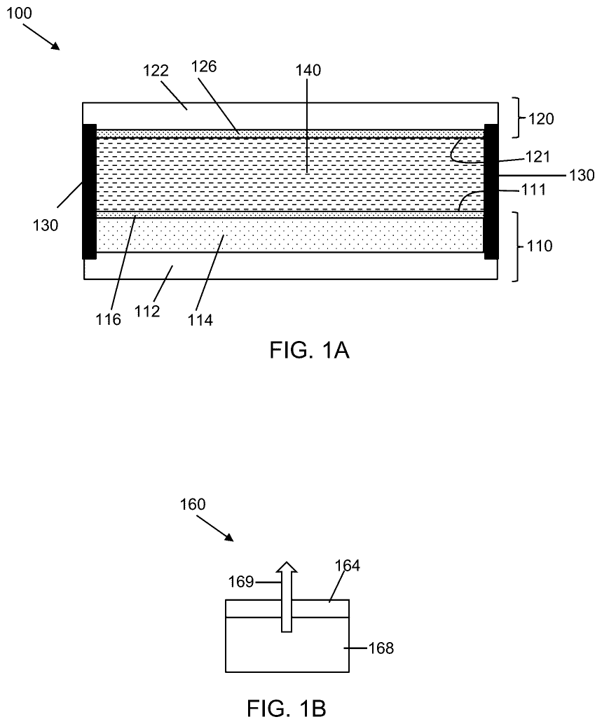

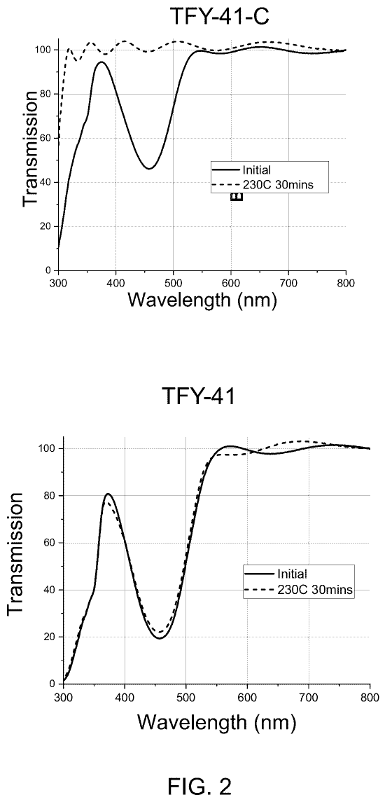

One example of a device is a liquid crystal cell as shown cross-sectional schematic view in FIG. 1A. Here, the liquid crystal cell 100 includes a first cell plate 110 having a top surface 111. The first cell plate includes a first transparent substrate 112 (e.g., glass); disposed on the first transparent substrate, the colored dielectric polymer material 114; and disposed on the colored dielectric polymer material, a first transparent conducting oxide film 116 (e.g., ITO), the transparent conducting oxide being within 100 nm of the top surface of the first cell plate. In this example, the first transparent conducting oxide film 116 forms the fop surface of the first cell plate, but the person or ordinary skill in the art will appreciate that one or more thin layers of other materials may be provided on the conducting oxide surface. The liquid crystal cell 100 also includes a second cell plate 120 having a top surface 121. The second cell plate includes a second transparent substrate 122 (e.g., glass); disposed on the second transparent substrate, a second transparent conducting oxide film 126 (e.g., ITO), the transparent conducting oxide being within 100 nm of the top surface of the second cell plate. Here, too, one or more thin layers of other materials may be provided on the conducting oxide surface. Moreover, a colored dielectric film can be provided in the second cell plate, in much the same way as in the first cell plate. One or more spacers 130 are disposed between the top surface of the first cell plate and the top surface of the second cell plate, the one or more spacers defining lateral edges of the liquid crystal cell. And a liquid crystal material 140 is disposed in a volume defined by the top surface of the first cell plate, the top surface of the second cell plate, and the one or more spacers.

As the person of ordinary skill in the art will appreciate, optical properties of the liquid crystal material can be adjusted by adjusting a potential between the first and second conductive oxide layers. The optical properties of the liquid crystal can, in turn, determine whether light passes through the system. As the person of ordinary skill in the art will appreciate, in a conventional liquid crystal display the polarization of the liquid crystal can be tuned by the potential applied between the first and second conductive oxide layers, thus controlling the transmittance of the light between two 90° aligned polarizer films. Other liquid crystal display types can be used as well, such as reflective type LCDs.

But the materials described herein can be used in a variety of other devices, for example, to provide colored light (e.g., as from a colored pixel). One embodiment of such a device is shown in schematic view in FIG. 1B. In device 160, a colored dielectric polymer material of the disclosure 164 is operatively coupled to a light source 168, configured to filter light emanating from the light source in a display direction 169. The light source can be, e.g., a relatively large light source, with a number of different colored film sections of colored dielectric polymer material, e.g., as part of a liquid crystal display, or can be configured as a single LED pixel (e.g., a single OLED) pixel, with a LED or OLED source and a section of a colored material of the disclosure. In various embodiments, the device can optionally include conductive oxide layer, as described above, formed on or adjacent the colored dielectric film. The materials described herein can also be adapted to provide a color filter for ambient light with respect to an observer.

In fact, the present disclosure provides for a variety of devices that include a colored dielectric polymer material in contact with a transparent conducting oxide. As demonstrated here, the materials of the disclosure are surprisingly robust to the deposition and annealing conditions used to make such conductive oxides.

In another aspect, the present disclosure provides methods for making a device as otherwise described herein. In certain embodiments, the method comprises: forming a film of the colored dielectric polymer material; depositing a transparent conducting oxide electrode through sputtering adjacent the film of the colored dielectric polymer material (e.g., on the film); and annealing at a temperature of at least 200° C. (e.g., at least 220° C.) for a time in the range of 10 minutes to 24 hours.

Photolithography is a process for patterning electronic components. Accordingly, in certain embodiments as otherwise described herein, the method of making a device further comprises a photolithography process. In particular embodiments, the photolithography process comprising: applying a photoresist layer; irradiation through a patterned photo mask, developing the patterned photoresist layer, dry-etching the exposed underneath film and stripping the remaining photoresist layer (positive photolithography process). Alternatively, the photolithography process can be accomplished by directly exposing the organic film under irradiation through a patterned photo mask, and develop the un-crosslinked film with an organic solvent (negative photolithography process). Either photolithography process can, in certain embodiments result in a pattern with a resolution of no more than 10 μm (e.g., no more than 8 μm), and wherein the intensity of the maximum transmittance changes by no more than 20% following the photolithography process.

### Definitions

Terms used herein may be preceded and/or followed by a single dash, “—”, or a double dash, “═”, to indicate the bond order of the bond between the named substituent and its parent moiety; a single dash indicates a single bond and a double dash indicates a double bond or a pair of single bonds in the case of a spiro-substituent. In the absence of a single or double dash it is understood that a single bond is formed between the substituent and its parent moiety; further, substituents are intended to be read “left to right” with reference to the chemical structure referred to unless a dash indicates otherwise. For example, arylalkyl, arylalkyl-, and -alkylaryl indicate the same functionality.

For simplicity, chemical moieties are defined and referred to throughout primarily as univalent chemical moieties (e.g., alkyl, aryl, etc.) or divalent chemical moieties (e.g., alkylene, alkenylene). Nevertheless, such terms are also used to convey corresponding multivalent moieties under the appropriate structural circumstances clear to those skilled in the art. For example, while an “alkyl” moiety can refer to a monovalent radical (e.g., CH₃—CH₂—), in some circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene). All atoms are understood to have their normal number of valences for bond formation (i.e., 4 for carbon, 3 for N, 2 for O, and 2, 4, or 6 for S, depending on the oxidation state of the S). Nitrogens in the presently disclosed compounds can be hypervalent, e.g., an N-oxide or tetrasubstituted ammonium salt. On occasion a moiety may be defined, for example, as —B-(A)ₐ, wherein a is 0 or 1. In such instances, when a is 0 the moiety is —B and when a is 1 the moiety is —B-A.

As used herein, a “polymer” or “polymeric compound” refers to a molecule (e.g., a macromolecule) including a plurality of repeating units connected by covalent chemical bonds. A polymer can be represented by the general formula:

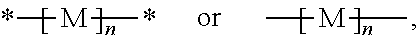

wherein M is the repeating unit or monomer, and n is the number of M's in the polymer. The polymer or polymeric compound can have only one type of repeating unit as well as two or more types of different repeating units. In the former case, the polymer can be referred to as a homopolymer. In the latter case, the term “copolymer” or “copolymeric compound” can be used instead, especially when the polymer includes chemically significantly different repeating units. The polymer or polymeric compound can be linear or branched. Branched polymers can include dendritic polymers, such as dendronized polymers, hyperbranched polymers, brush polymers, and the like. Unless specified otherwise, the assembly of the repeating units in the copolymer can be head to tail, head to head, or tail to tail. In addition, unless specified otherwise, the copolymer can be a random copolymer, an alternating copolymer, or a block copolymer. In some embodiments, formulae similar to the one below can be used to represent a copolymer, and such formula should be interpreted to embrace a copolymer having any repeating pattern consisting of x⁰ % of M¹, y⁰ % of M², and z⁰ % of M³, where M¹, M², and M³ are different repeating units:

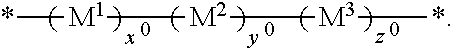

That is, the order and orientation of M¹, M² and M³ are not intended to be specific, and the formula is intended to encompass alternating, random, and block copolymers of M¹, M², and M³.

As used herein, a “pendant group” or “side group” is part of a repeating unit of a polymer and refers to a moiety that is attached covalently to the backbone of the polymer. As used herein, a “photopolymer” refers to a polymer that can be cured, for example, crosslinked by exposure to light, often using light in the ultraviolet region of the spectrum, or other types of radiation.

As used herein, “solution-processable” refers to polymers, materials, or composition that can be used in various solution-phase processes including spin-coating, printing (e.g., inkjet printing, screen printing, pad printing, offset printing, gravure printing, flexographic printing, lithographic printing, mass-printing and the like), spray coating, electrospray coating, drop casting, slot-die coating, dip coating, and blade coating. “Solution processable” also includes dispersion of polymers, materials, or compositions as long as they can be processes by the processes mentioned above.

As used herein, “halo” or “halogen” refers to fluoro, choro, bromo, or iodo. As used herein, “oxo” refers to a double-bonded oxygen (i.e., ═O).

As used herein, “alkyl” refers to a straight-chain or branched saturated hydrocarbon group. Examples of alkyl groups include methyl, ethyl, propoyl (e.g., n-propyl or iso-propyl), butyl (e.g., n-butyl, iso-butyl, sec-butyl, tert-butyl), pentyl groups (e.g., n-pentyl, iso-pentyl, neopentyl), hexyl groups, and the like. In various embodiments, an alkyl group can have 1 to 40 carbon atoms (i.e., C₁₋₄₀ alkyl group), for example, 1-20 carbon atoms (i.e., C₁₋₂₀ alkyl group). In some embodiments, an alkyl group can have 1 to 6 carbon atoms, and can be referred to as a “lower alkyl group.” Examples of lower alkyl groups include methyl, ethyl, propyl (e.g., n-propyl and iso-propyl), butyl (e.g., n-butyl, iso-butyl, sec-butyl, tert-butyl), pentyl groups (e.g., n-pentyl, iso-pentyl, neopentyl), and hexyl groups. In some embodiments, alkyl groups can be substituted as otherwise described herein.

As used herein, “haloalkyl” refers to an alkyl group having one or more halogen substituents. At various embodiments, a haloalkyl group can have 1 to 40 carbon atoms (i.e., C₁₋₄₀ haloalkyl group), for example, 1 to 20 carbon atoms (i.e., C1-₂₀ haloalkyl group). Examples of haloalkyl groups include CF₃, C₂F₅, CHF₂, CH₂F, CC1₃, CHC1₂, CH₂C1, C2Cl5, and the like. Perhaloalkyl groups, i.e., alkyl groups where all of the hydrogen atoms are replaced with halogen atoms (e.g., CF₃ and C₂F₅), are included within the definition of “haloalkyl.” For example, a C₁₋₄₀ haloalkyl group can have the formula —C₂H_{2z+1-t}X⁰ₜ, where X⁰, at each occurrence, is F, Cl, Br or I, z is an integer in the range of 1 to 40, and t is an integer in the range of 1 to 81, provided that t is less than or equal to 2z+1. Haloalkyl groups that are not perhaloalkyl groups can be substituted as described herein.

As used herein, “alkoxy” refers to —O-alkyl group. Examples of alkoxy groups include, but are not limited to, methoxy, ethoxy, propoxy (e.g., n-propoxy and isopropoxy), t-butoxy, pentoxyl, hexoxyl groups, and the like. The alkyl group in the —O-alkyl group can be substituted as described herein.

As used herein, “alkylthio” refers to an —S-alkyl group. Examples of alkylthio groups include, but are not limited to, methylthio, ethylthio, propylthio (e.g., n-propylthio and isopropylthio), t-butylthio, pentylthio, hexylthio groups, and the like. The alkyl group in the —S— alkyl group can be substituted as described herein.

As used herein, “alkenyl” refers to a straight-chain or branched alkyl group having one or more carbon-carbon double bonds. Examples of alkenyl groups include ethenyl, propenyl, butenyl, pentenyl, hexenyl, butadienyl, pentadienyl, hexadienyl groups, and the like. The one or more carbon-carbon double bonds can be internal (such as in 2-butene) or terminal (such as in 1-butene). In various embodiments, an alkenyl group can have 2 to 40carbon atoms (i.e., C₂-4o alkenyl group), for example, 2 to 20 carbon atoms (i.e., C2-20alkenyl group). In some embodiments, alkenyl groups can be substituted as described herein. An alkenyl group is generally not substituted with another alkenyl group, an alkyl group, or an alkynyl group.

As used herein, “alkynyl” refers to a straight-chain or branched alkyl group having one or more triple carbon-carbon bonds. Examples of alkynyl groups include ethynyl, propynyl, butynyl, pentynyl, hexynyl, and the like. The one or more triple carbon-carbon bonds can be internal (such as in 2-butyne) or terminal (such as in 1-butyne). In various embodiments, an alkynyl group can have 2 to 40 carbon atoms (i.e., C₂₋₄₀ alkynyl group), for example, 2 to 20 carbon atoms (i.e., C₂₋₂₀ alkynyl group). In some embodiments, alkynyl groups can be substituted as described herein. An alkynyl group is generally not substituted with another alkynyl group, an alkyl group, or an alkenyl group.

As used herein, “cyclic” refers to an organic closed-ring group including cycloalkyl groups, aryl groups, cycloheteroalkyl groups, and heteroaryl groups as defined herein.

As used herein, “cycloalkyl” refers to a non-aromatic carbocyclic group including cyclized alkyl, cyclized alkenyl, and cyclized alkynyl groups. In various embodiments, a cycloalkyl group can have 3 to 40 carbon atoms (i.e., C3-40 cycloalkyl group), for example, 3to 20 carbon atoms. A cycloalkyl group can be monocyclic (e.g., cyclohexyl) or polycyclic (e.g., containing fused, bridged, and/or spiro ring systems), where the carbon atoms are located inside the ring system. Any suitable ring position of the cycloalkyl group can be covalently linked to the defined chemical structure. Examples of cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcaryl, adamantyl, and spiro[4.5]decanyl groups, as well as their homologs, isomers, and the like. In some embodiments, cycloalkyl groups can be substituted as described herein.

As used herein, “heteroatom” refers to an atom of any element other than carbon or hydrogen and includes, for example, nitrogen, oxygen, silicon, sulfur, phosphorus, and selenium.

As used herein, “cycloheteroalkyl” refers to a non-aromatic cycloalkyl group that contains at least one ring heteroatom selected from O, S, Se, N, P, and Si (e.g., O, S, and N), and optionally contains one or more double or triple bonds. A cycloheteroalkyl group can have 3 to 40 ring atoms (i.e., 3-40 membered cycloheteroalkyl group), for example, 3 to 20ring atoms. One or more N, P, S, or Se atoms (e.g., N or S) in a cycloheteroalkyl ring may be oxidized (e.g., morpholine N-oxide, thiomorpholine S-oxide, thiomorpholine S,S-dioxide). In some embodiments, nitrogen or phosphorus atoms of cycloheteroalkyl groups can bear a substituent, for example, a hydrogen atom, an alkyl group, or other substituents as described herein. Cycloheteroalkyl groups can also contain one or more oxo groups, such as oxopiperidyl, oxooxazolidyl, dioxo-(IH,3H)-pyrimidyl, oxo-2(IH)-pyridyl, and the like. Examples of cycloheteroalkyl groups include, among others, morpholinyl, thiomorpholinyl, pyranyl, imidazolidinyl, imidazolinyl, oxazolidinyl, pyrazolidinyl, pyrazolinyl, pyrrolidinyl, pyrrolinyl, tetrahydrofuranyl, tetrahydrothiophenyl, piperidinyl, piperazinyl, and the like. In some embodiments, cycloheteroalkyl groups can be substituted as described herein.

As used herein, “aryl” refers to an aromatic monocyclic hydrocarbon ring system or a polycyclic ring system in which two or more aromatic hydrocarbon rings are fused (i.e., having a bond in common with) together or at least one aromatic monocyclic hydrocarbon ring is fused to one or more cycloalkyl and/or cycloheteroalkyl rings. An aryl group can have 6 to 40 carbon atoms in its ring system, which can include multiple fused rings. In some embodiments, a polycyclic aryl group can have from 8 to 40 carbon atoms. Any suitable ring position of the aryl group can be covalently linked to the defined chemical structure. Examples of aryl groups having only aromatic carbocyclic ring(s) include phenyl, 1-naphthyl (bicyclic), 2-naphthyl (bicyclic), anthracenyl (tricyclic), phenanthrenyl (tricyclic), and like groups. Examples of polycyclic ring systems in which at least one aromatic carbocyclic ring is fused to one or more cycloalkyl and/or cycloheteroalkyl rings include, among others, benzo derivatives of cyclopentane (i.e., an indanyl group, which is a 5,6-bicyclic cycloalkyl/aromatic ring system), cyclohexane (i.e., a tetrahydronaphthyl group, which is a 6,6-bicyclic cycloalkyl/aromatic ring system), imidazoline (i.e., a benzimidazolinyl group, which is a 5,6-bicyclic cycloheteroalkyl/aromatic ring system), and pyran (i.e., a chromenyl group, which is a 6,6-bicyclic cycloheteroalkyl/aromatic ring system). Other examples of aryl groups include benzodioxanyl, benzodioxolyl, chromanyl, indolinyl groups, and the like. In some embodiments, aryl groups can be substituted as described herein. In some embodiments, an aryl group can have one or more halogen substituents, and can be referred to as a “haloaryl” group. Perhaloaryl groups, i.e., aryl groups where all of the hydrogen atoms are replaced with halogen atoms (e.g., —C₆F₅), are included within the definition of “haloaryl.” In certain embodiments, an aryl group is substituted with another aryl group and can be referred to as a biaryl group. Each of the aryl groups in the biaryl group can be substituted as disclosed herein.

As used herein, “heteroaryl” refers to an aromatic monocyclic ring system containing at least one ring heteroatom selected from oxygen (O), nitrogen (N), sulfur (S), silicon (Si), and selenium (Se) or a polycyclic ring system where at least one of the rings present in the ring system is aromatic and contains at least one ring heteroatom. Polycyclic heteroaryl groups include two or more heteroaryl rings fused together and monocyclic heteroaryl rings fused to one or more aromatic carbocyclic rings, non-aromatic carbocyclic rings, and/or non-aromatic cycloheteroalkyl rings. A heteroaryl group, as a whole, can have, for example, 5 to 40 ring atoms and contain 1-5 ring heteroatoms. The heteroaryl group can be attached to the defined chemical structure at any heteroatom or carbon atom that results in a stable structure. Generally, heteroaryl rings do not contain O—O, S—S, or S—O bond. However, one or more N or S atoms in a heteroaryl group can be oxidized (e.g., pyridine N-oxide, thiophene S-oxide, thiophene S,S-dioxide).

### EXAMPLES

The Examples that follow are illustrative of specific embodiments of the methods of the disclosure, and various uses thereof. They are set forth for explanatory purposes only, and are not to be taken as limiting the scope of the disclosure.

Chemical reagent palladium (II) acetate (Pd(OAc)₂), tri-tertbutyl phosphine (t-Bu₃P), sodium tert-butoxide (t-BuONa), potassium carbonate, hexamethylenediamine (4), 4,7,10-trioxa-1,13-tridecanediamine (8), diphenylamine, eugenol (11), 1-chloroanthraquinone (13), 4-tert-butylphenol (28), bisphenol M (23), benzoyl chloride, and thiophenol were purchased from Sigma Aldrich (Milwaukee, Wis., USA) and used as is without additional purification. PHEMA was purchased from Scientific Polymer Products Inc (Ontario, New York, USA). Coumaric acid (44) was purchased from Oakwood Products Inc (Estill, SC, USA). Anhydrous solvent dimethylformamide (DMF), N-methyl-2-pyrrolidone (NMP), and toluene were purchased from Sigma Aldrich (Milwaukee, Wis., USA). Solvent dichloromethane, methanol, hexane, and ethyl acetate were purchased from VWR (Radnor, PA, USA). Compound 6-bromobenzo [de] isochromene-1,3-dione (3), 4-chloro-1,8-naphthalic anhydride (20), and bis (4-(tert-butyl) phenyl) amine (32) were purchased from Ambeed Inc. (Arlington Heights, IL, USA). Reagents 1, 10, 27, and 42 were synthesized according to procedures that were reported in the literature (See (a) Tang, G. et al. J. Phys. Chem. C 2019, 123, 30171-30186; (b). Chao, C. C. et al. J. Org. Chem. 2005, 70, 4323-4331. (c) Schmidt, C. D. et al. Chem. Eur. J. 2011, 17, 5289-5299. (d) Tanaka, H. et al. J. Polym. Sci. Part A-1, 1972, 10, 1729-1743.). Conventional Schlenk techniques were used, and reactions were carried out under nitrogen or argon unless otherwise noted. Characterization data are provided in some cases by ¹H NMR, and optical absorption spectroscopy. NMR spectra were recorded on an Inova 500 NMR spectrometer (1H, 500 MHz). UV-vis spectra were recorded on a Cary 50 UV-vis spectrophotometer.

### Example 1: Synthesis of Dye-117

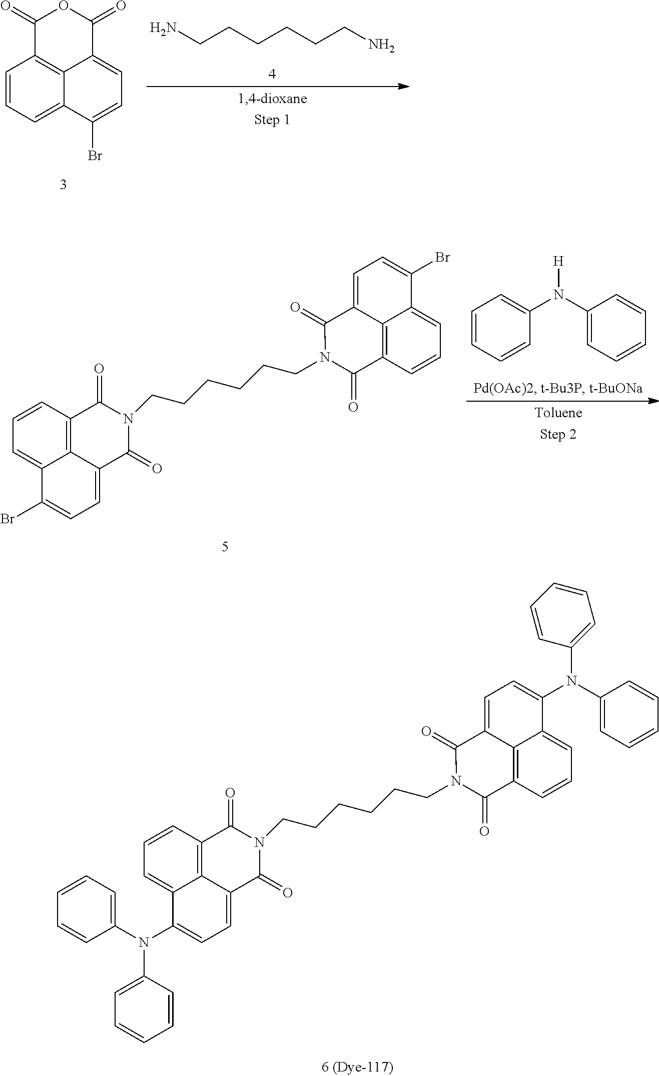

### Step 1

Under nitrogen, a mixture of compound 3 (6.82 g, 24.6 mmol), diamine compound 4 (1.43 g, 12.3 mmol) in 1,4-dioxane (50 mL) was stirred at refluxing for about 20 hours. Upon cooling to rt, the precipitates were collected by vacuum filtration, rinsed with 1,4-dioxane and methanol, and dried in vacuum, leading a pale-yellow solid as the product, which was used directly for next step without further purification (Compound 5, 6.85 g, 87.7%). ¹H NMR, (500 MHz, CDCl₃), δ(ppm): 8.63 (dd, J=7.5 Hz, J=1.0 Hz, 2H), 8.56 (dd, J=8.5 Hz, J=1.0 Hz, 2H), 8.39 (d, J=8.0 Hz, 2H), 8.03 (d, J=7.5 Hz, 2H), 7.84 (dd, J=8.5 Hz, J=7.0 Hz, 2H), 4.17 (tr, J=7.5 Hz, 4H), 1.75 (m, br, 4H), 1.50 (m, br, 4H).

### Step 2

Under Ar, a mixture of compound 5 (0.54 g, 0.85 mmol), diphenylamine (0.36 g, 2.13 mmol), Pd(OAc)₂ (67.3 mg, 0.30 mmol), t-Bu₃P (96.0 mg, 0.60 mmol), and t-BuONa (1.12 g, 11.7 mmol) in anhydrous toluene (40 mL) was heated to about 120° C. and maintained at this temperature for about 5 hours. Upon cooling to rt, water (˜40 mL) was added, followed by addition of ethyl acetate (˜100 mL). The mixture was stirred and separated. The water layer was extracted with ethyl acetate (˜100 mL). Organic layers were combined, washed with water and brine, dried over anhydrous sodium sulfate, and concentrated in vacuo. The residue was treated with methanol, and the solid product was collected by vacuum filtration, rinsed with methanol, and dried in vacuum, leading to a brownish-yellow solid as the product (Compound 6, Dye-117) (0.3 g, 43.5%). ¹H NMR, (500 MHz, CDCl₃), δ(ppm): 8.47-8.52 (m, br, 4H), 8.16 (d, J=8.5 Hz, 2H), 7.45-7.50 (dd, J=8.5 Hz, J=7.5 Hz, 2H), 7.36 (d, J=8.0 Hz, 2H), 7.23-7.30 (m, br, 10 Hz), 7.00-7.10 (m, br, 10H), 4.18 (tr, J=7.5 Hz, 4H), 1.76 (m, br, 4H), 1.50 (m, br, 4H).

### Example 2: Synthesis of Dye-115

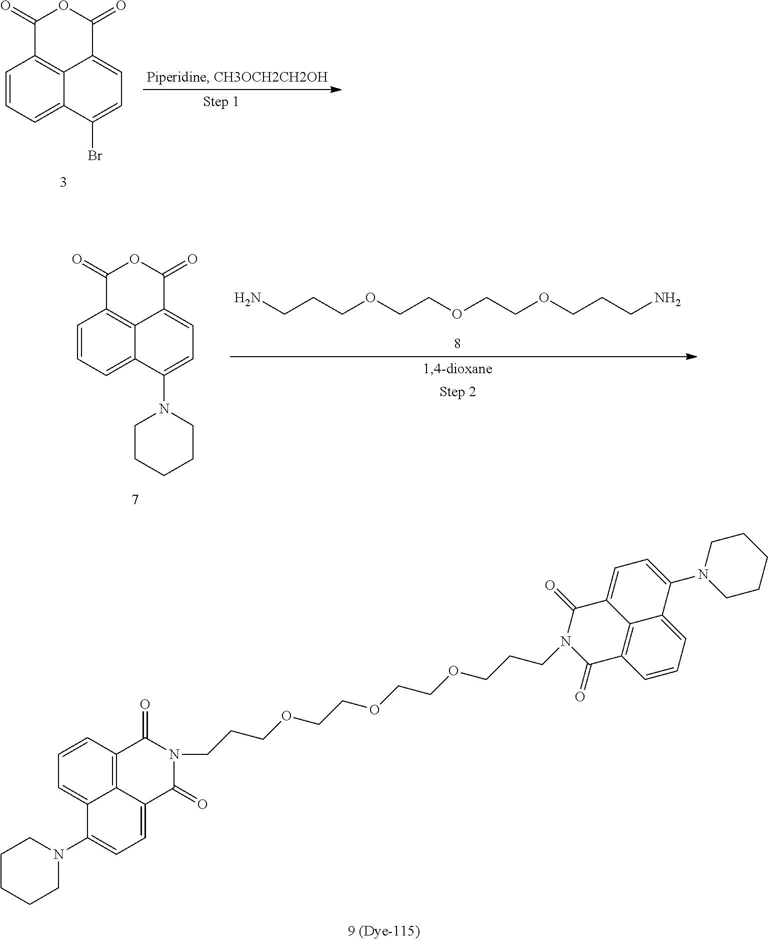 [SMILES: O=C1OC(=O)c2ccc(Br)c3cccc1c23 ; O=C1OC(=O)c2ccc(N3CCCCC3)c3cccc1c23 ; NCCCOCCOCCOCCCN ; O=C1c2cccc3c(N4CCCCC4)ccc(c23)C(=O)N1CCCOCCOCCOCCCN1C(=O)c2cccc3c(N4CCCCC4)ccc(c23)C1=O]

### Step 1

A mixture anhydride compound 3 (10.26 g, 37.0 mmol) and piperidine (7.5 mL, 75.9 mmol) in methoxyethanol (80 mL) was stirred under argon for about 7 hours. Upon cooling to rt, the yellow/orange precipitates were collected by vacuum filtration, rinsed with methanol, and dried in vacuum, leading to a yellow/orange solid as the product (7), which was used directly for next step without further purification (8.3 g, 79.7%). ¹H NMR, (500 MHz, CDCl₃), δ(ppm): 8.57 (dd, J=7.5 Hz, J=1.0 Hz, 1H), 8.50 (d, J=8.5 Hz, 1H), 8.42 (dd, J=8.5 Hz, J=1.0 Hz, 1H) 7.71 (dd, J=8.5 Hz, J=7.5 Hz, 1H), 7.20 (d, J=8.5 Hz, 1H), 3.29 (t, J=5.0 Hz, 4H,), 1.89 (m, br, 4H), 1.76 (m, br, 2H).

### Step 2

Under Ar, a mixture of compound 7 (3.26 g, 11.6 mmol), diamine compound 8 (1.22 g, 5.6 mmol) in 1,4-dioxane (25 mL) was stirred at refluxing for about 5 hours. Upon cooling to rt, the volatile was removed in vacuo and the residue was recrystallized from a mixture of ethyl acetate and methanol, leading to a yellow solid as the product (Compound 9 (Dye-115), 3.8 g, 91.8%). ¹H NMR, (500 MHz, CDCl₃), δ(ppm): 8.35-8.50 (m, br, 6H), 7.60 (m, br, 2H), 7.14 (m, br, 2H), 4.18 (tr, J=7.0 Hz, 24H), 3.40-3.54 (m, br, 12H), 3.19 (s, br, 8H), 1.94 (m, br, 4H), 1.80-1.90 (m, br, 8H), 1.66 (m, br, 4H).

### Example 3: Synthesis of Dye-27

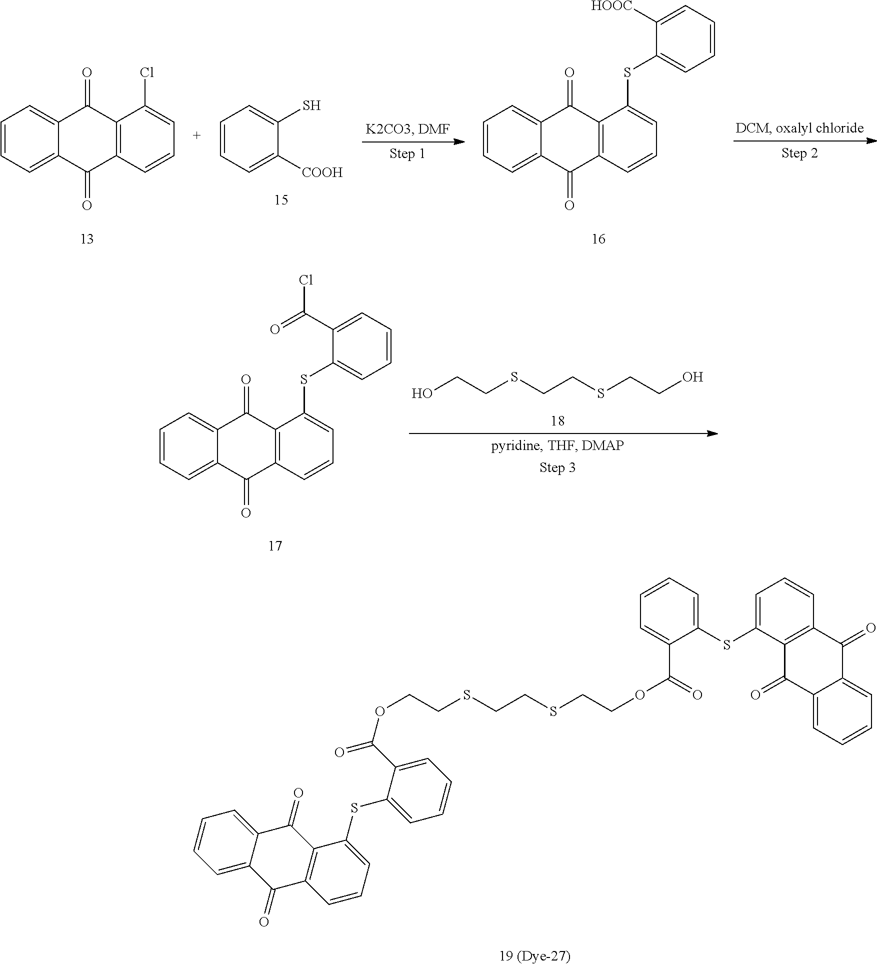

### Step 1

Under argon, a mixture of 1-chloroanthraquinone (compound 13) (12.1 g, 49.9 mmol), thiosalicylic acid 15 (7.7 g, 49.9 mmol), and potassium carbonate (7.0 g, 50.7 mmol) in DMF (100 mL) was stirred at 125° C. for about 6 hours. Upon cooling to rt, the reaction mixture was poured into water (˜200 mL), and the resulting mixture was stirred at rt for about 10 mins, before it was acidified by addition of acetic acid carefully (until pH ˜5). The precipitates were collected by filtration, washed with warm water, and dried in vacuum, leading to a greenish-yellow solid as the product (compound 16) (16.8 g, 93.5%). ¹H NMR, 500 MHz, (DMSO-D6), δ(ppm): 8.25 (d, J=7.5 Hz, 1H), 8.20 (d, J=7.0 Hz, 1H), 7.90-8.05 (m, br, 3H), 7.81 (d, J=6.0 Hz, 1H), 7.55-7.70 (m, br, 4H), 7.06 (d, J=8.0 Hz, 1H).

### Step 2

A mixture of compound 16 (2.3 g, 6.4 mmol) and oxalyl chloride (4 mL) in anhydrous DCM (100 mL) was stirred at rt for about 17 h, before all the volatiles were removed in vacuo. The residue was dried in vacuum, and it was then used directly for next step without further purification (Compound 17) (2.4 g, 99.2%). ¹H NMR, 500 MHz, (CD₂Cl₂), δ(ppm): 8.32 (dd, J=7.0 Hz, J=1.5 Hz, 1H), 8.26 (dd, J=7.5 Hz, J=1.5 Hz, 1H), 8.16 (m, br, 1H), 8.11 (dd, J=7.5 Hz, J=1.0 Hz, 1H), 7.78-7.86 (m, br, 2H), 7.74 (m, br, 1H), 7.63-7.70 (m, br, 2H), 7.48 (m, br, 1H), 7.02 (dd, J=8.0 Hz, J=10. Hz, 1H).

### Step 3

Under nitrogen, diol 18 (0.46 g, 2.51 mmol) and DMAP (41.3 mg, 0.34 mmol) were placed in an oven-dried flask. Dry pyridine (8 mL) was then added, followed by addition of a mixture of acyl chloride 17 (2.4 g, 6.34 mmol) in dry THF (18 mL). The resulting mixture was stirred at rt for 16 hours, before it was quenched by addition of methanol (3 mL). The reaction was stirred at rt for additional 45 mins, before it was concentrated in vacuo. The residue was taken with ethyl acetate (200 mL), and the resulting mixture was washed with water (150 mL×2), dried over anhydrous sodium sulfate, and concentrated to about 20 mL in vacuo. This residue was precipitated in methanol (˜200 mL). The precipitates were collected by vacuum filtration, relished with methanol, and dried in vacuum, leading to brownish-yellow solid as the product (Compound 19, Dye-27) (1.6 g, 73.4%). ¹H NMR, 500 MHz, (CDCl₃), δ(ppm): 8.35 (d, J=7.0 Hz, 2H), 8.27 (d, J=7.0 Hz, 2H), 8.09 (d, J=7.5 Hz, 2H), 7.92 (m, br, 2H), 7.80 (m, br, 4H), 7.70 (m, br, 2H), 7.58 (m, br, 4H), 7.45 (m, br, 2H), 7.05 (d, J=8.0 Hz, 2H), 4.25 (t, J=7.0 Hz, 4H), 2.61 (t, J=7.0 Hz, 4 H), 2.57 (s, 4H).

### Example 4: Synthesis of Dye-51

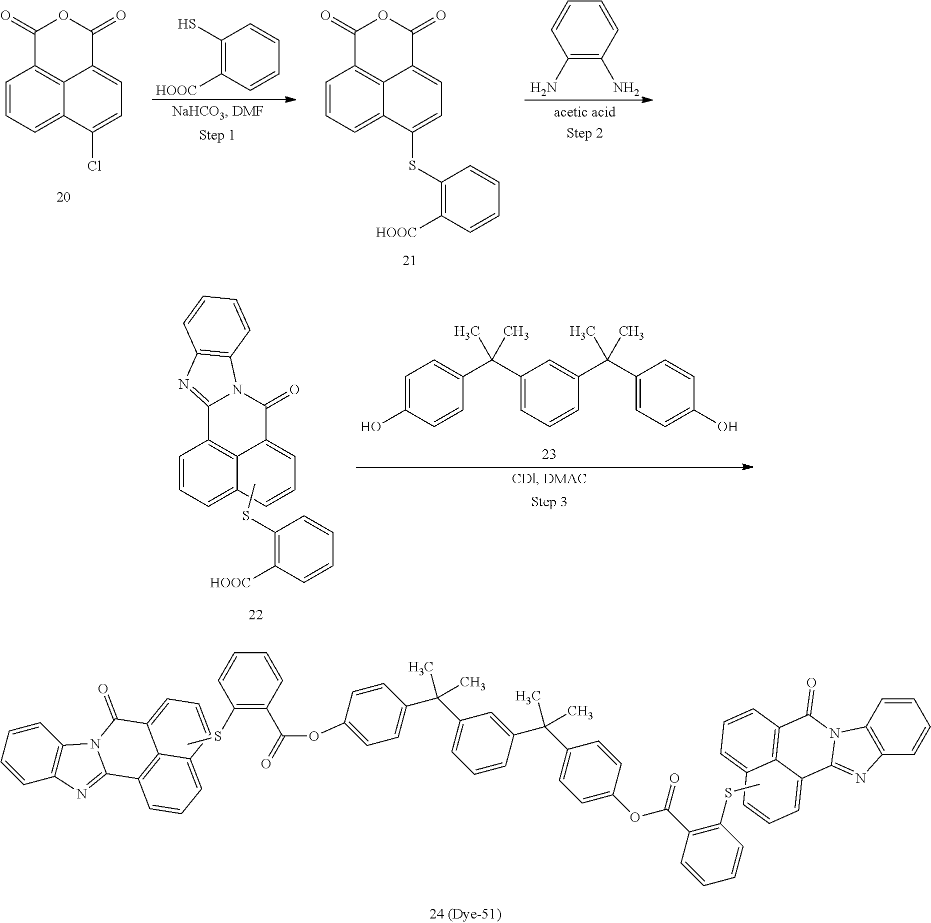

### Step 1

Under argon, a mixture of compound 20 (12.7 g, 54.5 mmol), thiosalicylic acid 15 (12.6 g, 81.9 mmol), and sodium bicarbonate (4.92 g, 58.6 mmol) in DMF (150 mL) was stirred at 150-152° C. for about 6.5 hours. Upon cooling to rt, the reaction mixture was carefully acidified by adding 5% HCl solution. The resulting precipitates were collected by filtration, washed with water, and dried in vacuum, leading to a pale-yellow solid as the product (compound 21) (18.7 g, 97.9%). ¹H NMR, 500 MHz, (DMSO-D6), δ(ppm): 8.62 (dd, J=8.5 Hz, J=1.0 Hz, 1H), 8.58 (dd, J=7.0 Hz, J=1.0 Hz, 1H), 8.50 (d, J=7.5 Hz, 1H), 7.90-8.05 (m, br, 3H), 7.35 (m, br, 2H), 6.75 (m, br, 1H).

### Step 2

A mixture of compound 21 (18.7 g, 53.4 mmol) and o-diaminobenzene (6.4 g, 58.7 mmol) in acetic acid (250 mL) was refluxed for about 7 h. Upon cooling to rt, the yellow solid was collected by vacuum filtration, rinsed with small portion of acetic acid and methanol, and dried in vacuum, leading to a yellow solid as the product (Compound 22 (two isomers not separated)) (18.7 g, 82.9%). ¹H NMR (mixture of two isomers), 500 MHz, (DMSO-D6), δ(ppm): 8.62-8.83 (m, br, 3H), 8.43-8.54 (m, br, 1H), 7.88-8.20 (m, br, 4H), 7.24-7.58 (m, br, 4H), 6.61-6.82 (m, br, 1H).

### Step 3

Under nitrogen, a mixture of compound 22 (1.0 g, 2.4 mmol) and CDI (0.39 g, 2.4 mmol) in dry DMAc was stirred at 70° C. for about 3.5 h. Compound bisphenol M (23) (0.40 g, 1.15 mmol) was added via dry DMAc (6 mL). The resulting mixture was stirred at 70° C. for about 16 hours. Upon cooling to rt, the reaction mixture was precipitated in a mixture of methanol (400 mL) and water (50 mL). The precipitates were collected by vacuum filtration, rinsed with water and methanol, and dried in vacuum, leading a yellow solid the product (Compound 24, Dye-51) (0.18 g, 13.5%).

### Example 5: Synthesis of Dye-82

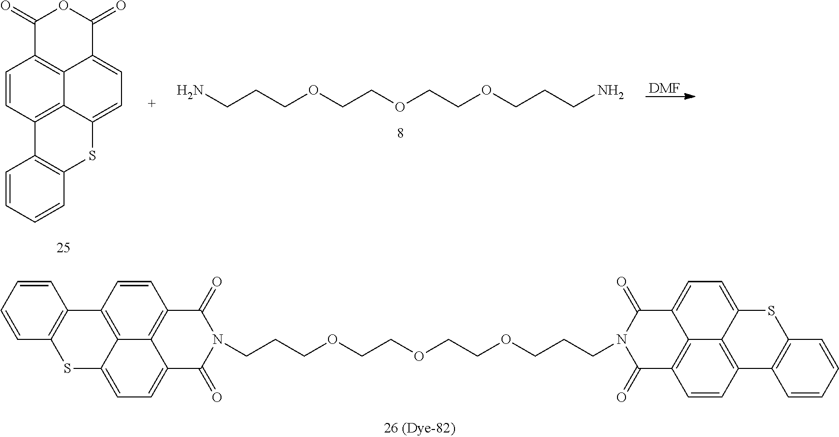

A solution of compound 25 (350 mg, 1.2 mmol) and 8 (121 mg, 0.5 mmol) in DMF (6 mL) was stirred at 100° C. under N₂ for 4 hours. The mixture was cooled to room temperature, precipitated into MeOH (30 mL), and collected by filtration. The solid was washed with MeOH (4×20 mL), EtOAc (4×20 mL) and then recrystallized from CHCl₃/EtOAc to give an orange solid as the product (Compound 26 (Dye-82)) (200 mg, 51%). ¹H NMR (500 MHz, CDCl₃), δ(ppm) 8.34 (d, 2H, J=8.18 Hz), 8.19 (d, 2H, J=8.0 Hz), 7.97 (m, 2H), 7.89 (d, 2H, J=8.33 Hz), 7.29 (m, 6H), 7.21 (m, 2H), 4.20 (t, 4H, J=7.35 Hz), 3.64 (m, 12H), 2.01 (m, 4H).

### Example 6: Synthesis of Dye-49

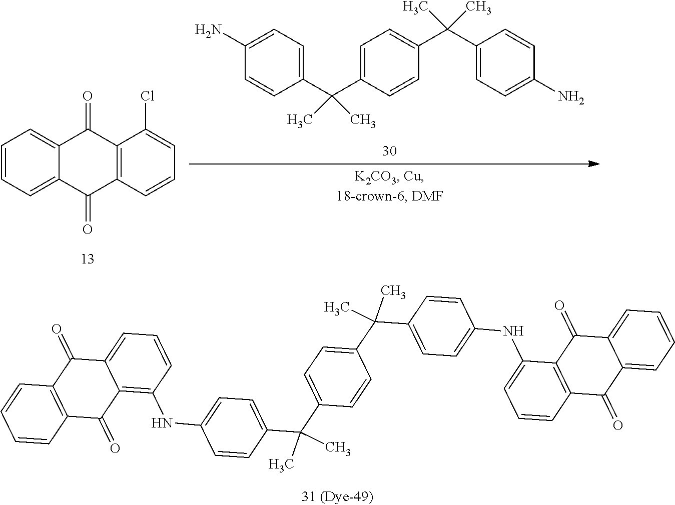 [SMILES: O=C1c2ccccc2C(=O)c2c(Cl)cccc21 ; CC(C)(c1ccc(N)cc1)c1ccc(C(C)(C)c2ccc(N)cc2)cc1 ; CC(C)(c1ccc(Nc2cccc3c2C(=O)c2ccccc2C3=O)cc1)c1ccc(C(C)(C)c2ccc(Nc3cccc4c3C(=O)c3ccccc3C4=O)cc2)cc1]

Under nitrogen, a mixture of compound 13 (12.5 g, 51.5 mmol), diamine compound 30 (4.45 g, 12.9 mmol), potassium carbonate (5.4 g, 39.1 mmol), copper (2.5 g, 39.3 mmol), 18-crown-6 (0.34 g, 1.3 mmol) in DMF (60 mL) was refluxed for about 20 hours. Upon cooling to rt, the insoluble material was filtered, and the filter cake was rinsed with small portion of DMF. The combined filtrate was precipitated in methanol (˜400 mL). The precipitates were collected by filtration, rinsed with water and methanol, dried in vacuum, leading to a red-purple solid as the product (Compound 31, (Dye-49)) (6.3 g, 64.4%). ¹H NMR, 500 MHz, (CDCl₃), δ(ppm): 11.4 (s, 2H), 8.39 (dd, J=8.5 Hz, J=2.0 Hz, 2H), 8.34 (dd, J=8.0 Hz, J=1.0 Hz, 2H), 7.76-7.88 (m, br, 6H), 7.58 (m, br, 4H), 7.27-7.39 (m, br, 12H), 1.78 (s, 12H).

### Example 7: Synthesis of Dye-200

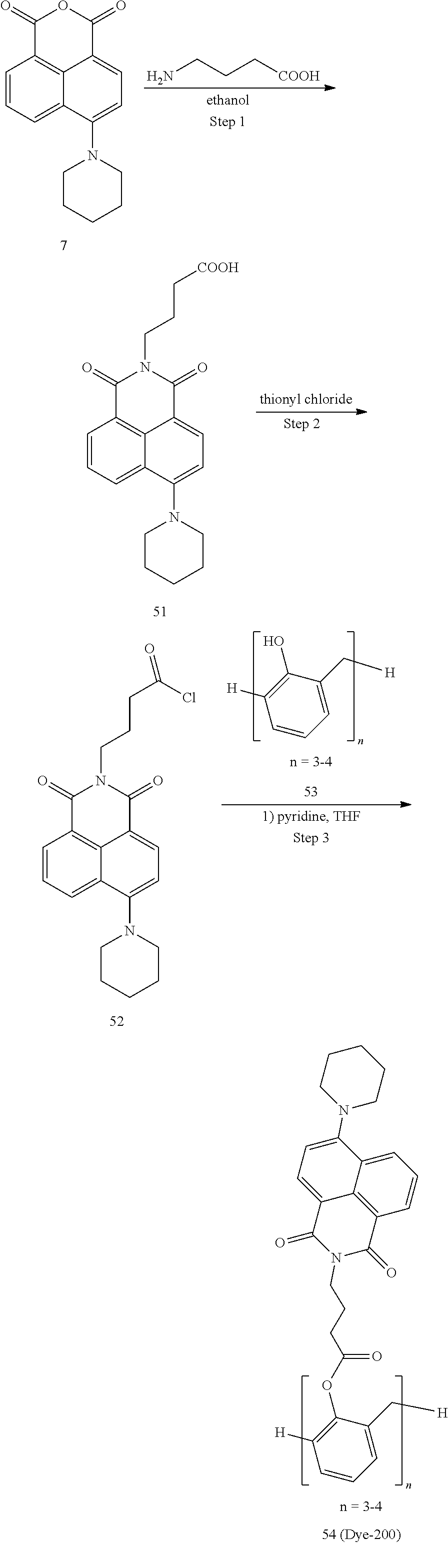

### Step 1

Under argon, a mixture of compound 7 (1.40 g, 4.98 mmol), and γ-aminobutyric acid (1.03 g, 9.99 mmol), in ethanol (50 mL) was stirred at refluxing for about 20 hours. Upon cooling to rt, most of solvent was removed in vacuo, and the residue was treated with DCM (40 mL). The insoluble material was filtered off, and filter cake was rinsed with DCM. The combined filtrate was purified by column chromatography on silica gel with a mixture of DCM: methanol=9:1 (v/v) as eluent, leading to a yellow solid as the product 51 (1.30 g, 71.4%). ¹H NMR, 500 MHz, (CDCl₃), (ppm): 8.50-8.72 (m, br, 3H), 7.76 (m, br, 1H), 7.36 (m, br, 1H), 4.26 (m, br, 2H), 3.37 (s, br, 4H), 2.48 (m, br, 2H), 1.85-2.18 (m, br 6H), 1.78 (m, br, 2H).

### Step 2

Under argon, a mixture of compound 51 (662.1 mg, 1.81 mmol) in thionyl chloride (20 mL) was stirred at rt for 4 h. Most of the volatiles were removed in vacuo, and the residue was dried in vacuum. This crude (52) was directly used for next step without further purification (701.3 mg).

### Step 3

Under nitrogen, phenolic reside 53 (153.7 mg, 1.45 mmol) was dissolved in anhydrous pyridine (10 mL), followed by addition of DMAP (5.3 mg, 0.043 mmol). Acyl chloride 52 (701.3 mg) in dry THF (10 mL) was then added via syringe. The resulting mixture was stirred at rt for about 5 hours, before it was poured into methanol (100 mL). The precipitates were collected by vacuum filtration, rinsed with methanol, and dried in vacuum, leading a yellow solid the product (54, Dye-138) (0.39 g, 59.2%). ¹H NMR, 500 MHz, (CDCl₃), δ(ppm): 8.00-8.65 (m, br, 3H), 7.64 (m, br, 1H), 6.30-7.40 (m, br, 4H), 3.00-4.40 (m, br, 6H), 1.40-2.70 (m, br, 12H).

### Example 8: Other Multimeric Dyes

The compounds shown below in Table 3 can be prepared essentially according to procedures known to those of skill in the art in view of above Examples 1-6.

TABLE 3

| Dye Name | Structure |
|---|---|
| Dye 145 | 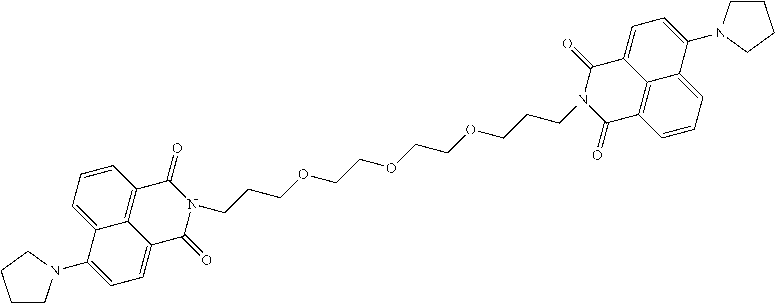 [SMILES: O=C1c2cccc3c(N4CCCC4)ccc(c23)C(=O)N1CCCOCCOCCOCCCN1C(=O)c2cccc3c(N4CCCC4)ccc(c23)C1=O] |
| Dye 121 | 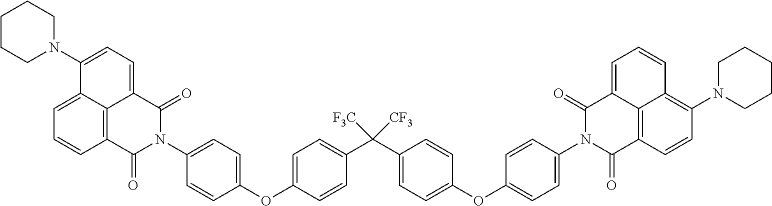 [SMILES: O=C1c2cccc3c(N4CCCCC4)ccc(c23)C(=O)N1c1ccc(Oc2ccc(C(c3ccc(Oc4ccc(N5C(=O)c6cccc7c(N8CCCCC8)ccc(c67)C5=O)cc4)cc3)(C(F)(F)F)C(F)(F)F)cc2)cc1] |
| Dye 41 | 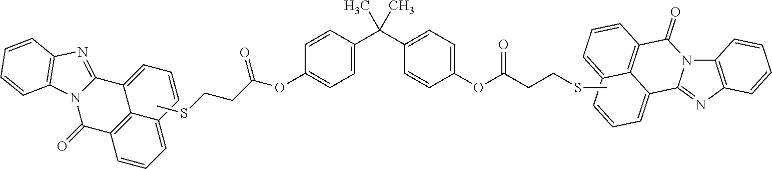 |
| Dye 3 | 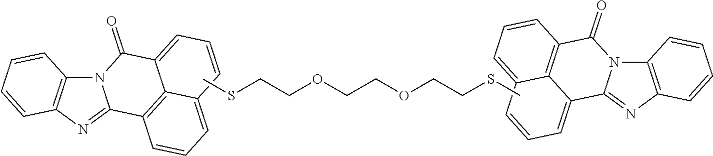 |
| Dye 35 | 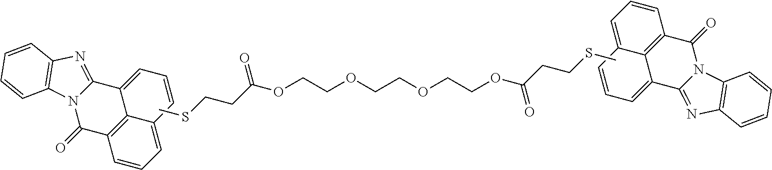 |
| Dye 86 | 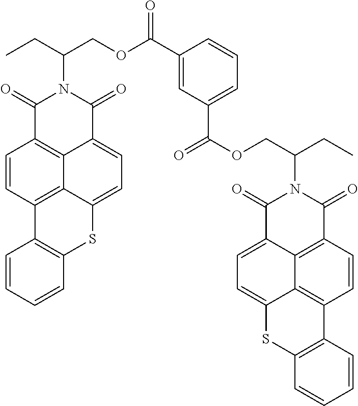 [SMILES: CCC(COC(=O)c1cccc(C(=O)OCC(CC)n2c(=O)c3ccc4sc5ccccc5c5ccc(c2=O)c3c45)c1)n1c(=O)c2ccc3sc4ccccc4c4ccc(c1=O)c2c34] |
| Dye 94 | 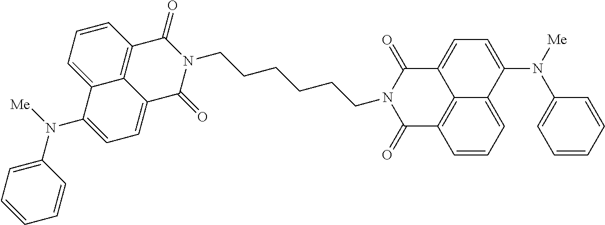 [SMILES: CN(c1ccccc1)c1ccc2c3c(cccc13)C(=O)N(CCCCCCN1C(=O)c3cccc4c(N(C)c5ccccc5)ccc(c34)C1=O)C2=O] |
| Dye 47 | 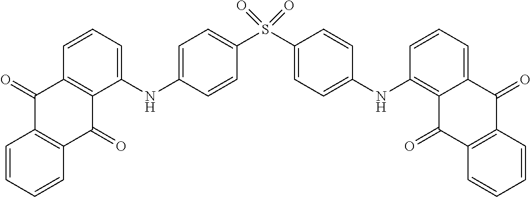 [SMILES: O=C1c2ccccc2C(=O)c2c(Nc3ccc(S(=O)(=O)c4ccc(Nc5cccc6c5C(=O)c5ccccc5C6=O)cc4)cc3)cccc21] |
| Dye 53 | 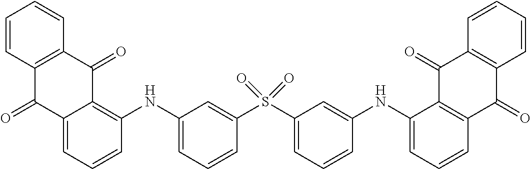 [SMILES: O=C1c2ccccc2C(=O)c2c(Nc3cccc(S(=O)(=O)c4cccc(Nc5cccc6c5C(=O)c5ccccc5C6=O)c4)c3)cccc21] |
| Dye 37 | 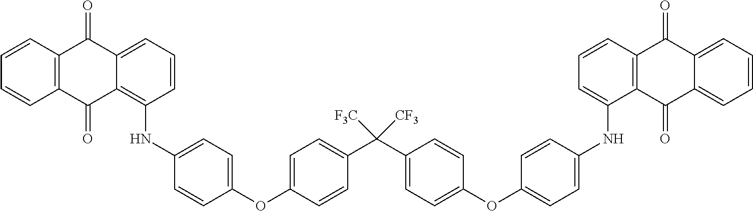 [SMILES: O=C1c2ccccc2C(=O)c2c(Nc3ccc(Oc4ccc(C(c5ccc(Oc6ccc(Nc7cccc8c7C(=O)c7ccccc7C8=O)cc6)cc5)(C(F)(F)F)C(F)(F)F)cc4)cc3)cccc21] |

### Example 9: Preparation of Colored dielectric Polymer Materials

Various dyes were synthesis as described in the above Examples, or purchased from commercial sources. Polymers were purchased (e.g., polysulfone) or can be prepared as described above or in International Patent Application publication nos. WO2013/199717 and WO2010/057984. M36 is a polymer of Formula 1a and B2000 is polymer of Formula b, having the structures:

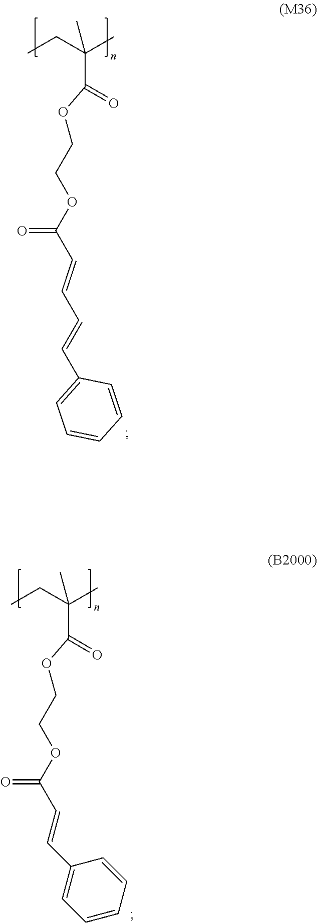

and described in the references cited herein.

The dye formulations (F's) were prepared by dissolving the polymer and the dye in a solvent with vigorous stirring at room temperature for about 4 hours. After dissolution, the formulation was filtered through a 0.2-1 um filter before use.

Formulations are described in Tables 4-6, below. FY, FR are formulations with yellow and red coloration, respectively. PGMEA is propylene glycol methyl ether acetate. CHN is cyclohexanone. TPO is triphenylphosphine oxide, TGE is trimethylolpropane triglycidyl ether. NIT is N-Hydroxynaphthalimide triflate.

TABLE 4
Formulation of Yellow Dyes

| | Polymer | | Solvent | |
|---|---|---|---|---|
| Name | (mg) | Dye (mg) | (mg) | Additive (mg) |
| FY-20 | PS (67) | Dye-115 (15) | CHN (947) | none |
| FY-21 | PS (67) | Dye-117 (52.5) | CHN (947) | none |
| FY-23 | M36 (130) | Dye-115 (40) | CHN (947) | none |
| FY-24 | PS (67) | Dye-126 (37) | Anisole (995) | none |
| FY-27 | PS (67) | Dye-145 (40) | CHN (947) | TGE (58) |
| | | | | NIT (2.5) |
| FY-34 | M36 (130) | Dye-27 (60) | CHN (947) | none |
| FY-35 | M36 (130) | Dye-51 (60) | Anisole (995) | none |
| FY-36 | M36 (67) | Dye-115 (34) | CHN (947) | none |
| FY-37 | M36 (200) | Dye-115 (50) | Anisole (995) | none |
| FY-38 | PS (67) | Dye-117 (50) | CHN (947) | none |

TABLE 5
Formulation of red dyes

| | Polymer | | | |
|---|---|---|---|---|
| Name | (mg) | Dye (mg) | Solvent (mg) | Additive (mg) |
| FR-20 | M36 (130) | Dye-37 (40) | Anisole (995) | none |
| FR-25 | M36 (130) | Dye-47 (40) | Anisole (995) | none |
| FR-26 | M36 (130) | Dye-53 (40) | Anisole (995) | none |
| FR-27 | M36 (130) | Dye-49 (50) | Anisole (995) | none |

TABLE 6
Dye materials used in Tables 4-5

| Dye | Chemical |
|---|---|
| name/ID | Structure |
| Dye-47 | 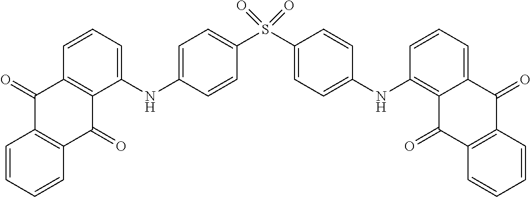 [SMILES: O=C1c2ccccc2C(=O)c2c(Nc3ccc(S(=O)(=O)c4ccc(Nc5cccc6c5C(=O)c5ccccc5C6=O)cc4)cc3)cccc21] |
| Dye-115 | 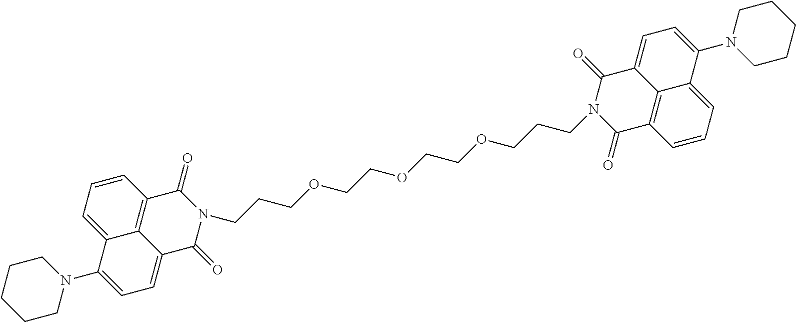 [SMILES: O=C1c2cccc3c(N4CCCCC4)ccc(c23)C(=O)N1CCCOCCOCCOCCCN1C(=O)c2cccc3c(N4CCCCC4)ccc(c23)C1=O] |
| Dye-117 | 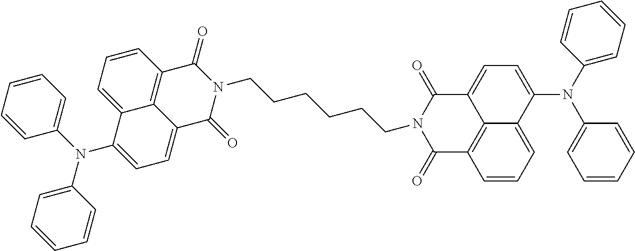 [SMILES: O=C1c2cccc3c(N(c4ccccc4)c4ccccc4)ccc(c23)C(=O)N1CCCCCCN1C(=O)c2cccc3c(N(c4ccccc4)c4ccccc4)ccc(c23)C1=O] |
| Dye-51 | 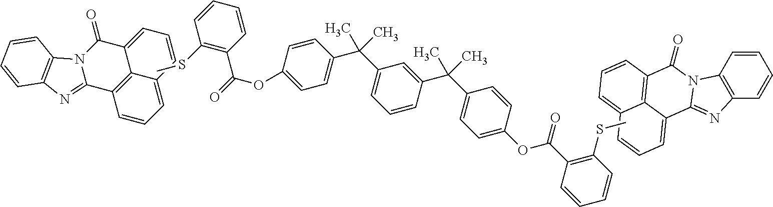 |
| Dye-126 | 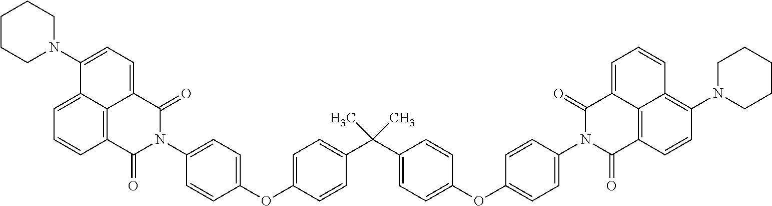 [SMILES: CC(C)(c1ccc(Oc2ccc(N3C(=O)c4cccc5c(N6CCCCC6)ccc(c45)C3=O)cc2)cc1)c1ccc(Oc2ccc(N3C(=O)c4cccc5c(N6CCCCC6)ccc(c45)C3=O)cc2)cc1] |
| Dye-53 | 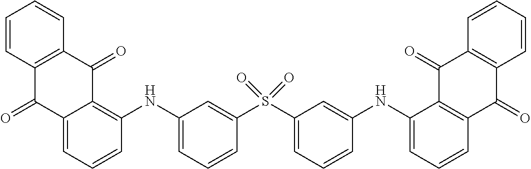 [SMILES: O=C1c2ccccc2C(=O)c2c(Nc3cccc(S(=O)(=O)c4cccc(Nc5cccc6c5C(=O)c5ccccc5C6=O)c4)c3)cccc21] |
| Dye-145 | 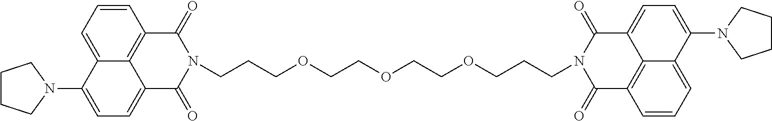 [SMILES: O=C1c2cccc3c(N4CCCC4)ccc(c23)C(=O)N1CCCOCCOCCOCCCN1C(=O)c2cccc3c(N4CCCC4)ccc(c23)C1=O] |
| Dye-49 | 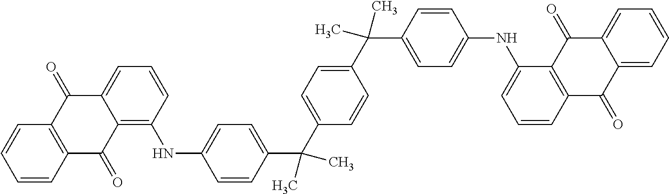 [SMILES: CC(C)(c1ccc(Nc2cccc3c2C(=O)c2ccccc2C3=O)cc1)c1ccc(C(C)(C)c2ccc(Nc3cccc4c3C(=O)c3ccccc3C4=O)cc2)cc1] |
| Dye-37 | 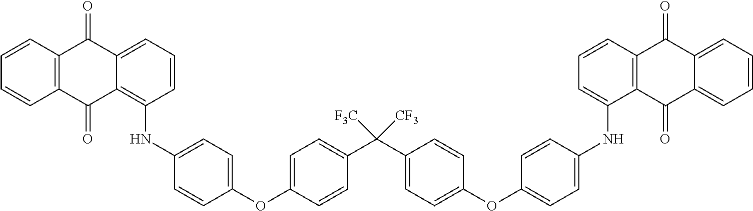 [SMILES: O=C1c2ccccc2C(=O)c2c(Nc3ccc(Oc4ccc(C(c5ccc(Oc6ccc(Nc7cccc8c7C(=O)c7ccccc7C8=O)cc6)cc5)(C(F)(F)F)C(F)(F)F)cc4)cc3)cccc21] |
| Dye-27 | 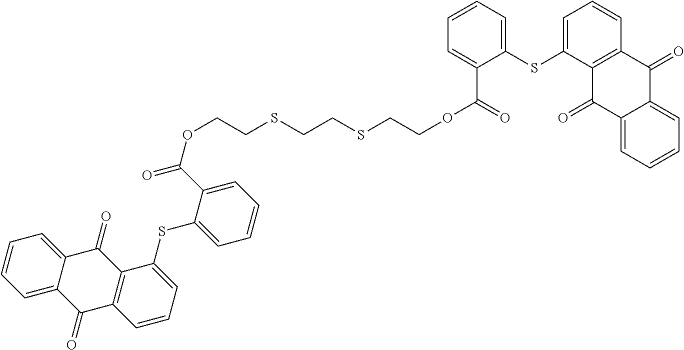 [SMILES: O=C(OCCSCCSCCOC(=O)c1ccccc1Sc1cccc2c1C(=O)c1ccccc1C2=O)c1ccccc1Sc1cccc2c1C(=O)c1ccccc1C2=O] |

### Example 10: Thin Film Preparation

Spin-coating: Dye thin films (TF's) were fabricated on Corning EAGLE glass or plastic (PEN) substrates. The substate was cleaned with acetone, soap water and IPA and exposed to air plasma prior to deposition. The formulation is spun-coated (400-2000 rpm) onto the substrate to afford, after a soft bake (80˜120° C., 1˜5 mins) on a hotplate, ˜500-3500 nm-thick films.

Slot-die coating: Dye thin films were fabricated on Corning EAGLE glass or plastic (PEN) substrates. The substate is cleaned with acetone, soap water and IPA and exposed to air plasma prior to deposition. The formulation was coated using an Ossilia slot-die coater (coating gap 100˜500 um, coating rate 1˜500 mm/s) onto the substrate to afford, after a soft bake (80˜120° C., 1˜5 mins) on a hotplate, ˜500-3500 nm-thick films.

Thickness of the dye thin films were measured using Dektek 150 profilometer. UV-Vis spectrum of the dye thin films were measured in air using Cary 50 UV-vis spectrophotometer.

Detail of the thin films (TF's) are collected in Tables 7-8:

TABLE 7
Thin films of yellow dyes on glass substrates

| | | | Formu- | | | Transmittance | |
|---|---|---|---|---|---|---|---|
| Name | Substrate | Method | lation | Thickness | | (@ xx nm) | |
| TFY-20 | glass | spin | FY-20 | 542 | nm | 48% | (408 nm) |
| TFY-21 | glass | spin | FY-21 | 685 | nm | 17% | (454 nm) |
| TFY-23 | glass | spin | FY-23 | 1.05 | um | 4% | (408 nm) |
| TFY-24 | glass | spin | FY-24 | 762 | nm | 20% | (410 nm) |
| TFY-27 | glass | spin | FY-27 | 1.04 | um | 3% | (447 nm) |
| TFY-34 | glass | spin | FY-34 | 1.88 | um | 22% | (437 nm) |
| TFY-35 | glass | spin | FY-35 | 1.18 | um | 8% | (417 nm) |
| TFY-36 | glass | spin | FY-36 | 560 | nm | 40% | (410 nm) |
| TFY-37 | glass | spin | FY-37 | 1.20 | um | 22% | (414 nm) |
| TFY-38 | glass | spin | FY-23 | 980 | nm | 9% | (410 nm) |
| TFY-39 | glass | spin | FY-23 | 740 | nm | 18% | (410 nm) |
| TFY-41 | glass | spin | FY-38 | 675 | nm | 19% | (452 nm) |

TABLE 8
Thin films of red dyes on glass substrate

| | | | | | Transmittance |
|---|---|---|---|---|---|
| Name | Substrate | Method | Formulation | Thickness | (@ xx nm) |
| TFR-20 | glass | spin | FR-20 | 1.08 um | 44% (511 nm) |
| TFR-25 | glass | spin | FR-25 | 1.02 um | 36% (504 nm) |
| TFR-26 | glass | spin | FR-26 | 1.02 um | 45% (503 nm) |
| TFR-27 | glass | spin | FR-27 | 1.24 um | 27% (523 nm) |

### Example 11: Thermal Stability Comparison

To assess the thermal stability of a film containing a multimeric dye as compared to a film containing a monomeric analogue, polysulfone films including monomeric dye 59 and dimeric dye 117 (see Table 9, below, for structures) were subjected to thermal annealing conditions, and their optical properties compared before and afterwards. The film including dye 59 (TFY-41-C) was 812 nm in thickness and was cast from a solution including 15 mg dye 59, 67 mg polysulfone, and 947 mg cyclohexanone (CHN). The film including dye 117 (TFY-41) was 675 nm in thickness and was cast from a solution FY-38 (50 mg dye 117, 67 mg polysulfone and 947 mg CHN). Both films were annealed on a hotplate at 230° C. for 30 minutes in ambient atmosphere.

TABLE 9

| Dye Name | Structure |
|---|---|
| Dye-59 | 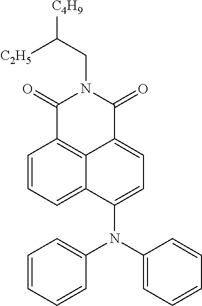 |
| Dye-117 | |

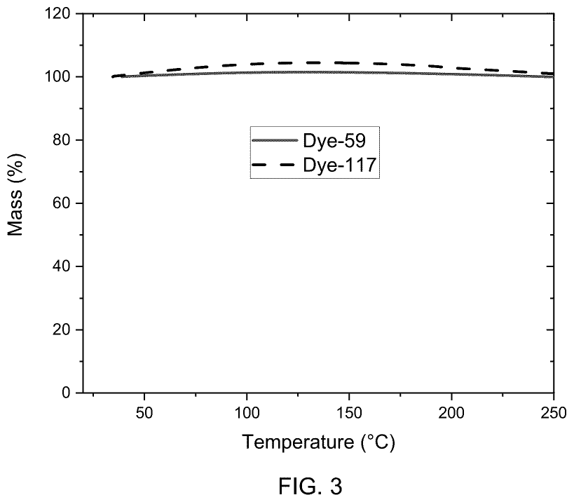

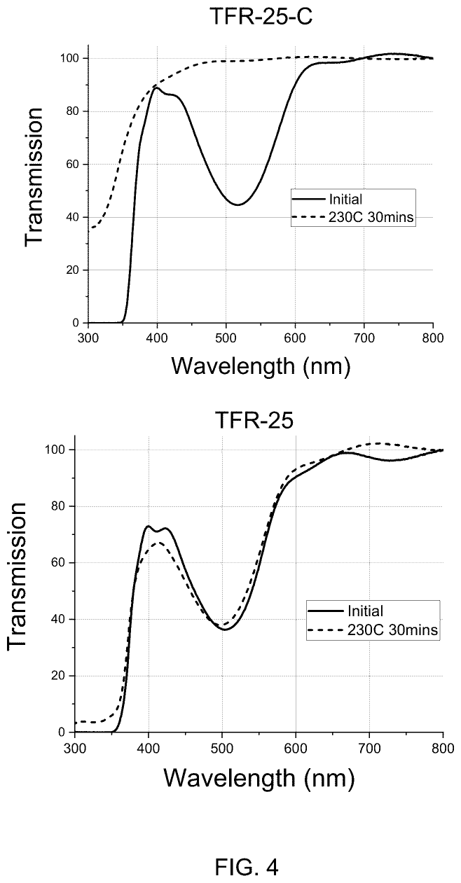

FIG. 2 displays the transmission spectra of the films before and after thermal treatment. It can be seen that the film including dye 59 displays a marked variation of the optical transmission properties, whereby the transmission minimum at ˜460 nm is greatly reduced, demonstrating a fading of color after thermal treatment. In contrast, the transmittance of the color film based on the dye 117 is stable to thermal treatment. FIG. 3 provides the thermogravimetric analysis (TGA) traces of dyes 59 and 117 as pure solid samples, demonstrating that both dyes themselves have substantially similar thermal properties and sublimation behavior in the same temperature range. Thus, it is surprising that when mixed in the polymer the two dyes behave so differently. Without intending to be bound by theory, it is believed that the different behavior is mainly due to entanglement of the connector between the two chromophores with the polymer chains that stabilizes the film at high temperature rather than an obvious increase of the molecular mass.

Similarly M36 polymer films containing monomeric dye LB (1-(phenylamino)anthracene-9,10-dione) and dimeric dye-47 (see Table 10, below, for structures) were compared. The film containing LB (TFR-25-C) was 922 nm in thickness and was cast from a solution of LB (25 mg) and M36 polymer (130 mg) in CHN (947 mg). The film containing Dye-47 (TFR-25) was 1023 nm in thickness and was cast from solution FR25 (Dye-47 40 mg and M36 polymer 130 mg in anisole 995 mg).

TABLE 10

| Dye Name | Structure |
|---|---|
| LB | 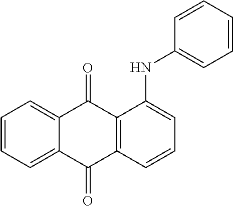 [SMILES: O=C1c2ccccc2C(=O)c2c(Nc3ccccc3)cccc21] |
| Dye-47 | 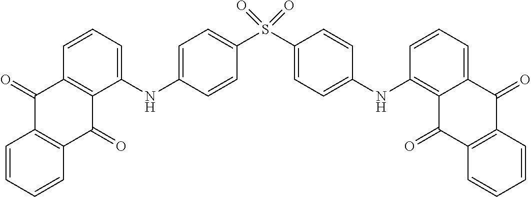 [SMILES: O=C1c2ccccc2C(=O)c2c(Nc3ccc(S(=O)(=O)c4ccc(Nc5cccc6c5C(=O)c5ccccc5C6=O)cc4)cc3)cccc21] |

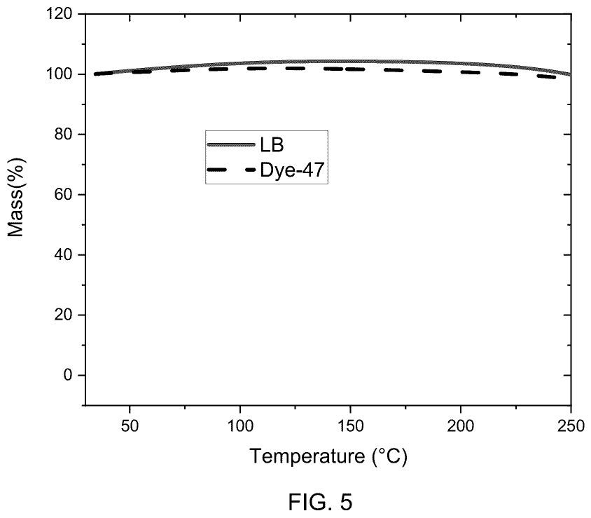

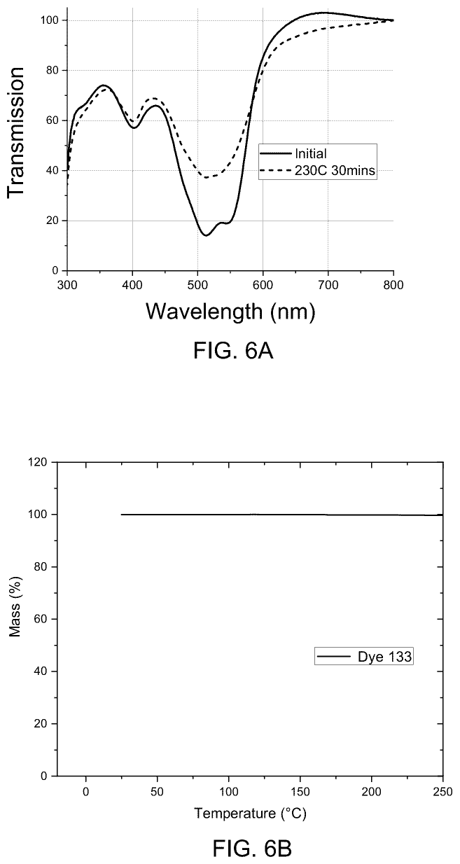

FIG. 4 displays the transmission spectra of the films before and after thermal treatment, and FIG. 5 provides a TGA trace of the dyes. As can be seen, the film including dye LB displays a marked shift in transmission properties, whereby the local transmission minimum at ˜515 nm is greatly reduced, indicating a fading of color, whereas the transmittance of the color film based on the dye-47 is stable. FIG. 5 provides the TGA traces of LB and dye-47 as pure solid samples, demonstrating that both dyes have identical thermal properties and sublimation behavior in the same temperature range. Thus, it is surprising that when mixed in the polymer the two dyes behave so differently.

In addition, films containing monomeric dye-133 (molecular weight ˜827 g/mole) and dye-MS165 (molecular weight ˜865 g/mole) were prepared. The film containing dye-133 (TFR-C2) was 605 nm in thickness and was cast from a solution of dye-133 (40 mg) and polysulphone (67 mg) in CHN (947 mg). The film containing dye-MS147 (TFG-C3) was 1650 nm in thickness and was cast from a solution of dye-MS165 (80 mg) and M36 polymer (120 mg) in CHN (947 mg).

TABLE 11

| | | Molecular |
|---|---|---|
| | | Weight |
| Dye Name | Structure | (g/mole) |
| Dye-117 | 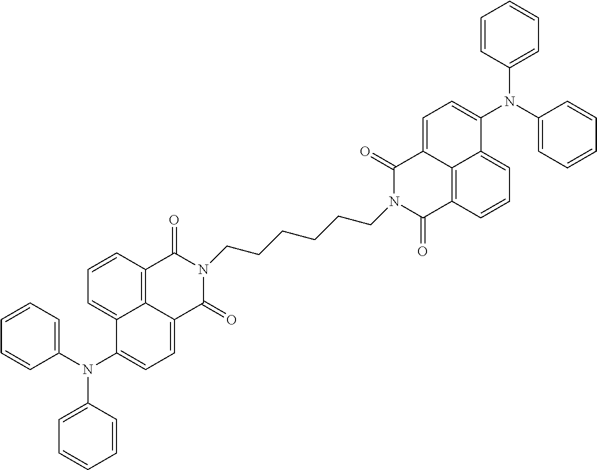 [SMILES: O=C1c2cccc3c(N(c4ccccc4)c4ccccc4)ccc(c23)C(=O)N1CCCCCCN1C(=O)c2cccc3c(N(c4ccccc4)c4ccccc4)ccc(c23)C1=O] | 811 |
| Dye-133 | 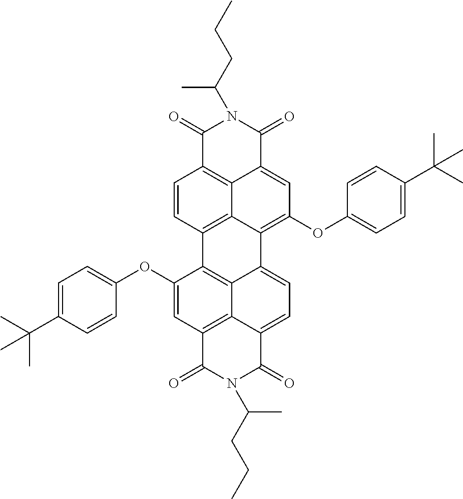 [SMILES: CCCC(C)N1C(=O)c2ccc3c4c(Oc5ccc(C(C)(C)C)cc5)cc5c6c(ccc(c7c(Oc8ccc(C(C)(C)C)cc8)cc(c2c37)C1=O)c64)C(=O)N(C(C)CCC)C5=O] | 827 |
| Dye-MS165 | 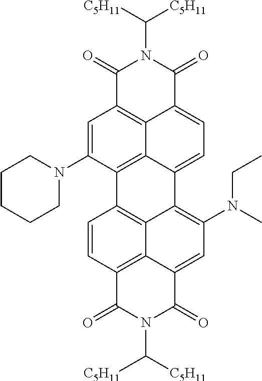 | 865 |

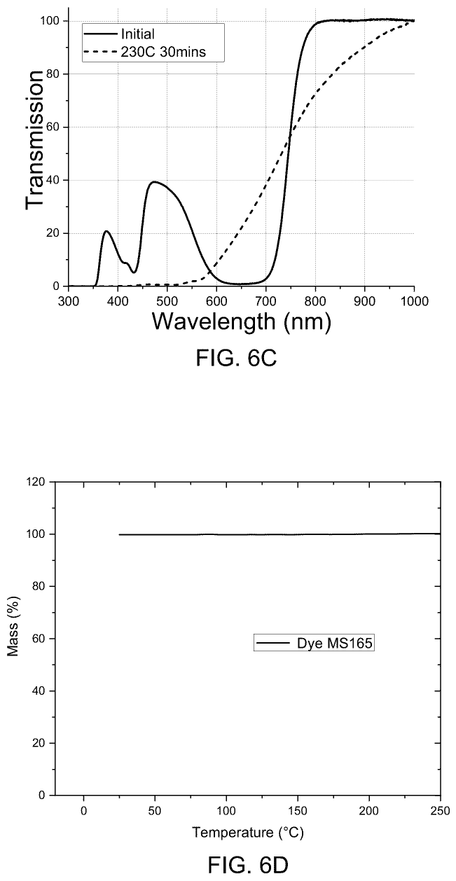

FIG. 6A displays the transmission spectra of TFR-25-C2 films before and after thermal treatment. FIG. 6B provides a TGA trace of the dye-133. FIG. 6C displays the transmission spectra of TFR-25-C3 films before and after thermal treatment, and FIG. 6D provides a TGA trace of the dye-MS165. As can be seen, the film including dye-133 displays a marked shift in transmission properties, whereby the local transmission minimum at ˜510 nm is greatly reduced, indicating a fading of color. Similarly, the film including dye-MS165 displays a marked shift in transmission properties, whereby the local transmission minimum at ˜650 nm is greatly reduced, indicating a fading of color. This contrasts with thin film including dimeric dye-117 with similar molecular weight (TFY-41) which is stable upon annealing (FIG. 4), although the TGA traces indicate the three dyes have identical thermal properties and sublimation behavior in the same temperature range.

### Example 12: Characterization of Thin Films, Thermal Resistance

Thermal resistance: The thin films were annealed at 230° C. for 30 min. The transmittance spectra were measured and compared before and after thermal annealing. Table 11 collects representative data. Note that TFY-41-C and TFR-25-C represents monomeric dyes as described in Example 11 for comparative purposes

TABLE 12
Thermal stability of selected dyes on glass substrate

| | | |
|---|---|---|
| | Transmittance (%) | Transmittance (%) |
| Film | before annealing | after annealing |

| | | | | |
|---|---|---|---|---|
| TFY-21 | 17% | (@454 nm) | 21% | (@454 nm) |
| TFY-23 | 4% | (@ 408 nm) | 5% | (@ 408 nm) |
| TFY-34 | 22% | (@437 nm) | 25% | (@438 nm) |
| TFY-35 | 8% | (@417 nm) | 12% | (@415 nm) |
| TFY-41 | 19% | (@455 nm) | 22% | (@455 nm) |
| TFY-41-C | 46% | (@457 nm) | 99% | (@457 nm) |
| TFR-20 | 44% | (@511 nm) | 45% | (@511 nm) |
| TFR-25 | 36% | (@504 nm) | 38% | (@499 nm) |
| TFR-25-C | 54% | (@514 nm) | 100% | (@514 nm) |
| TFR-26 | 45% | (@503 nm) | 47% | (@498 nm) |
| TFR-27 | 27% | (@523 nm) | 28% | (@521 nm) |

### Example 13: Characterization of Thin Films, Photolithography Resistance

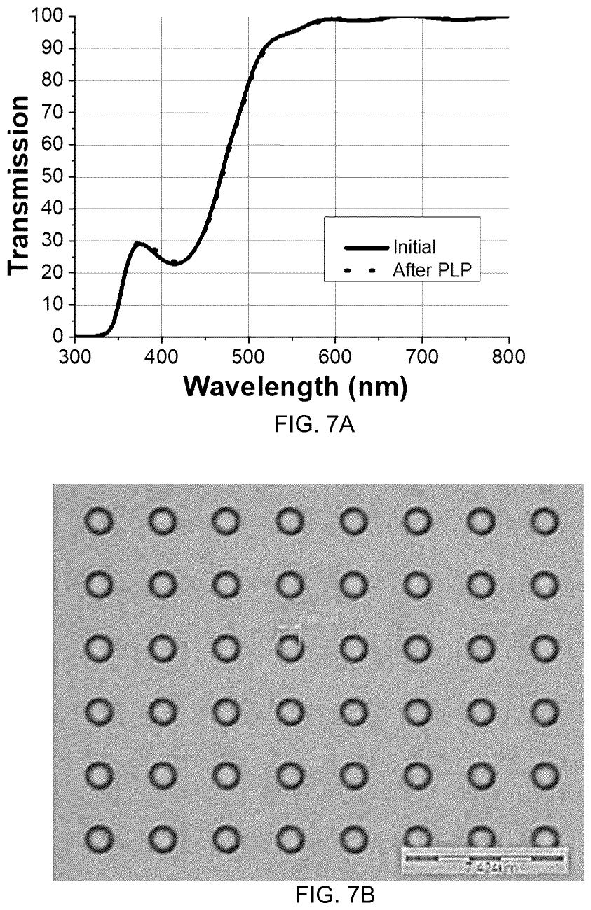

Photolithography resistance: The thin films were annealed at 230° C. for 30 min. The AZ650 photoresist solution was spun coated (2000 rpm, 120 mins) on the thin film of the dye and soft baked at 80 C 1 mins, followed by standard light exposure (GH-line, 30 mJ/cm2), development (TMAH 2.38%, 1 min), Ar and O₂ dry etching and stripping (N300 at 80° C. for 2 min) processes to define via holes in the color films. The transmittance spectra were measured and compared before and after the photolithographic process. Table 13 collects representative data. FIG. 7A displays UV-Vis spectra of a typical patterned film (Film TFY-37) before and after AZ photolithographic process (PLP) via hole of patterned TFY-37 film with dimensions ˜5 um, according to example embodiments. FIG. 7B provides a photomicrograph of the hole-patterned film.

TABLE 13
Stability to the AZ photolithographic process (PLP)
of the indicated films dyes on glass substrate.

| | Transmittance (%) | Transmittance (%) |
|---|---|---|
| Film | before PLP | after PLP |
| TFY-37 | 22% (@414 nm) | 22% (@414 nm) |
| TFR-26 | 47% (@498 nm) | 48% (@500 nm) |

### Example 14: Characterization of Thin Films, ITO Fabrication Stability

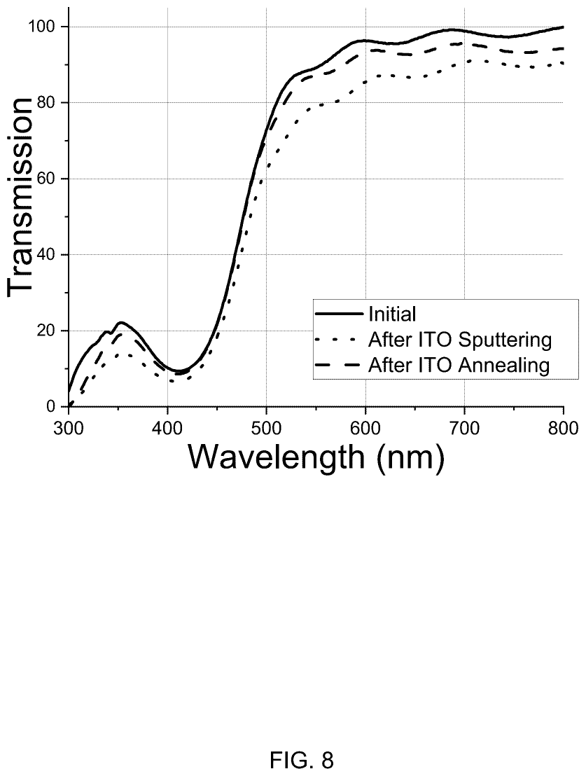

Stability to ITO fabrication process. The thin films were annealed at 230° C. for 30 min. The ITO film was deposited by sputtering (thickness 20˜120 nm) followed by annealing in a N2 oven at 230° C. for 30 mins. The transmittance spectra were measured and compared before and after the ITO fabrication process. Table 14 collects representative data. FIG. 8 displays UV-Vis spectra of a typical film (Film TFY-38) before and after ITO fabrication process according to example embodiments.

TABLE 14
Stability to the ITO fabrication process (ITO-FP)
of the indicated films dyes on glass substrate.

| | | |
|---|---|---|
| | Transmittance (%) | Transmittance (%) |
| Film | before ITO-FP | after ITO-FP |

| | | | |
|---|---|---|---|
| TFY-38 | 9% | (@410 nm) | 10% (@410 nm) |
| TFR-27 | 28% | (@521 nm) | 29% (@519 nm) |

### Example 14: Characterization of Thin Films, Stability to Light Exposure

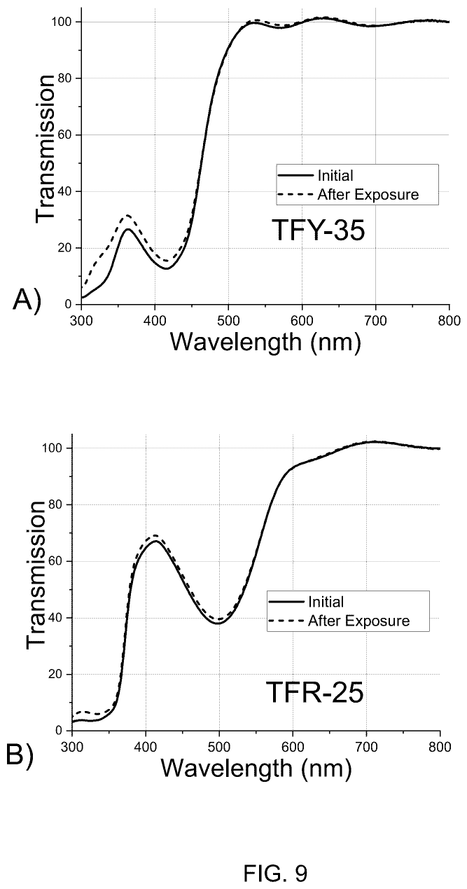

Light fastness: The thin films were annealed at 230° C. for 30 min and exposed to artificial sun light (Newport Solar Simulator 91160, 274 W) for 2 hr. The transmittance spectra were recorded and compared before and after light irradiation. Table 15 collects representative data. FIGS. 9A and 9B display UV-Vis spectra of thin films TFY-35 (9A) and TFR-25 (9B) before and after solar light exposure (SLE) according to example embodiments.

TABLE 15
Stability to solar light exposure (SLE) of the
indicated films dyes on glass substrate.

| | Transmittance (%) | Transmittance (%) |
|---|---|---|
| Film | before SLE | after SLE |
| TFY-35 | 12% (@415 nm) | 15% (@415 nm) |
| TFR-25 | 38% (@499 nm) | 40% (@500 nm) |

### Example 15: Characterization of Thin Films Dielectric Strength

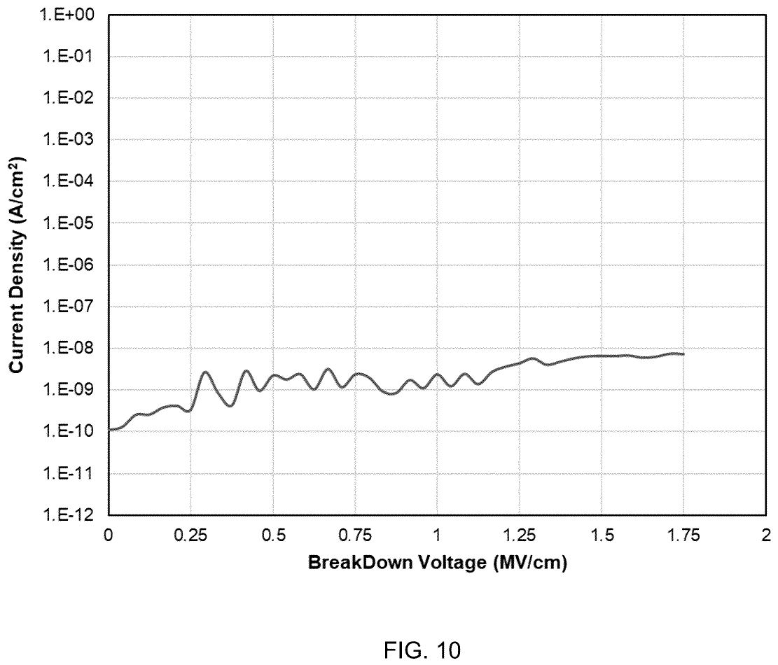

Dielectric strength measurements. Metal-Insulator-Metal (MIM) devices were used to investigate the dielectric strength of the thin films. The MIM bottom electrodes were fabricated by sputtering of Ag (100 nm) on a glass substrate and patterned by photolithography to yield circle-shaped electrodes with diameters of 100˜500 um. Thin films were deposited as describe in Example 10 affording ˜500-3500 nm films. The top electrodes were fabricated by sputtering of Ag (100 nm) on the thin film and patterned by photolithography to yield circle-shaped electrodes with diameters of 100˜500 um. Leakage current and breakdown voltages were measured using a probe station and a Keithley 4200electrometer. Table 16 collects representative data. FIG. 10 displays the leakage current measured from a typical thin film (TFY-37), according to an example embodiment.

TABLE 16
Dielectric strength of the indicated films dyes on glass substrate.

| | Leakage current (A/cm², | Breakdown Voltage | Dielectric |
|---|---|---|---|
| Film | @ 1 MV/cm) | (MV/cm) | Constant |
| TFY-37 | <10⁻⁸ | >1.5 | 3.0 |
| TFR-27 | <10⁻⁸ | >1.5 | 3.1 |

As can be seen by the foregoing Examples, the thin films fabricated according to the present disclosure possess excellent process stability, especially thermal stability, while maintaining good coloration characteristics.

Various exemplary embodiments of the disclosure include, but are not limited to the enumerated embodiments of the claims as listed below, which can be combined in any number and in any combination that is not technically or logically inconsistent.

The particulars shown herein are by way of example and for purposes of illustrative discussion of certain embodiments of the present disclosure only and are presented in the cause of providing what is believed to be the most useful and readily understood description of the principles and conceptual aspects of various embodiments of the disclosure. In this regard, no attempt is made to show details associated with the methods of the disclosure in more detail than is necessary for the fundamental understanding of the methods described herein, the description taken with the examples making apparent to those skilled in the art how the several forms of the methods of the disclosure may be embodied in practice. Thus, before the disclosed processes and devices are described, it is to be understood that the aspects described herein are not limited to specific embodiments, apparatus, or configurations, and as such can, of course, vary. It is also to be understood that the terminology used herein is for the purpose of describing particular aspects only and, unless specifically defined herein, is not intended to be limiting.

The terms “a,” “an,” “the” and similar referents used in the context of describing the methods of the disclosure (especially in the context of the following embodiments and claims) are to be construed to cover both the singular and the plural, unless otherwise indicated herein or clearly contradicted by context.

All methods described herein can be performed in any suitable order of steps unless otherwise indicated herein or otherwise clearly contradicted by context. The use of any and all examples, or exemplary language (e.g., “such as”) provided herein is intended merely to better illuminate the methods of the disclosure and does not pose a limitation on the scope of the disclosure. No language in the specification should be construed as indicating any non-claimed element essential to the practice of the methods of the disclosure.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

As will be understood by one of ordinary skill in the art, each embodiment disclosed herein can comprise, consist essentially of or consist of its particular stated element, step, ingredient or component. As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment.

All percentages, ratios and proportions herein are by weight, unless otherwise specified.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

Groupings of alternative elements or embodiments of the disclosure are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Some embodiments of various aspects of the disclosure are described herein, including the best mode known to the inventors for carrying out the methods described herein. Of course, variations on these described embodiments will become apparent to those of ordinary skill in the art upon reading the foregoing description. The skilled artisan will employ such variations as appropriate, and as such the methods of the disclosure can be practiced otherwise than specifically described herein. Accordingly, the scope of the disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

The phrase “at least a portion” as used herein is used to signify that, at least, a fractional amount is required, up to the entire possible amount.

In closing, it is to be understood that the various embodiments herein are illustrative of the methods of the disclosures. Other modifications that may be employed are within the scope of the disclosure. Thus, by way of example, but not of limitation, alternative configurations of the methods may be utilized in accordance with the teachings herein. Accordingly, the methods of the present disclosure are not limited to that precisely as shown and described.

### Embodiments

Embodiment 1. A multimeric dye having the structure:

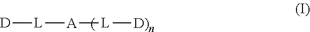

wherein:
each D is independently in isolated chromophore;
each L is independently absent or is a linking group that is no more than 10 atoms in length as measured in the shortest path from D to A;
A is a bridging group; and
n is an integer in the range of 1 to 3.

Embodiment 2. The multimeric dye of embodiment 1, wherein n is 1.

Embodiment 3. The multimeric dye of embodiment 1, wherein n is 2.

Embodiment 4. The multimeric dye of embodiment 1, wherein n is 3.

Embodiment 5. The multimeric dye of embodiment 2 or embodiment 3, wherein
A is:
C₁-C₁₀ alkylene or C₂-C₁₀ alkenylene, wherein optionally 1-6 carbon atoms of the backbone of each alkylene and/or alkenylene are each independently replaced by O, N(R^{A})₀₋₁, Si(R^{A})₀₋₂, or S;
monocyclic or spiro and/or fused polycyclic ring system, wherein the ring system comprises cycloalkyl, heterocycloalkyl, aromatic, and/or heteroaromatic rings, and wherein the ring system comprises no more than five total rings, and wherein the ring system optionally comprises 1-3 internal bridge groups X, wherein each X is independently a bond, —C(R^{A})(R^{B})—, —O—, —N(R^{A})₀₋₁—, —Si(R^{A})₀₋₂—, —S(O)₀₋₂—, or —C(R^{A})(R^{B})—C(R^{A}R^{B})—; or
polysiloxane, wherein each Si is optionally substituted with 1-2 methyl groups, or 1-2 phenyl groups, and comprises no more than 10 Si atoms;
wherein
each alkylene, alkenylene, cycloalkyl, heterocycloalkyl, aryl, and heteroaryl is independently and substituted with 0-5 R^{A}
R^{A} and R^{B} are each independently selected in each occurrence from H, methyl, ethyl, propyl, F, Cl, Br, oxo where chemically appropriate, —CF₃, or —OCH₃.

Embodiment 6. The multimeric dye of embodiment 4, wherein A is:
C₄-C₄₀ alkylene or C₆-C₄₀ alkenylene, wherein optionally 1-6 carbon atoms on the backbone of each alkyl and/or alkenyl are each independently replaced by O, N(R^{A})₀₋₁, or S; or
a monocyclic or spiro and/or fused polycyclic ring system, wherein the ring system comprises cycloalkyl, heterocycloalkyl, aromatic, and/or heteroaromatic rings, and wherein the ring system comprises no more than six total rings, and wherein the ring system optionally comprises 1-3 internal bridge groups X, wherein each X is independently a bond, —C(R^{A})₀₋₁(R^{B})₀₋₁—, —O—, —N(R^{A})₀₋₁—, —Si(R^{A})₀₋₂—, —S(O)₀₋₂—, or —C(R^{A})(R^{B})—C(R^{A})(R^{B})—; and
each alkylene, alkenylene, cycloalkyl, heterocycloalkyl, aryl, and heteroaryl is independently and substituted with 0-5 R^{A}.
wherein
each alkylene, alkenylene, cycloalkyl, heterocycloalkyl, aryl, and heteroaryl is independently and substituted with 0-5 R^{A}
R^{A} and R^{B} are each independently selected in each occurrence from H, methyl, ethyl, propyl, F, Cl, Br, oxo where chemically appropriate, —CF₃, or —OCH₃.

Embodiment 7. The multimeric dye of embodiment 1, wherein n is 1, and A is C₁-C₈ alkylene, —(CH₂CH₂—O)ₚCH₂CH₂—, —(CH₂CH₂Si(R^{A})₂)ₚCH₂CH₂—, or —(CH₂CH₂—N(R^{A}))ₚCH₂CH₂—, wherein p is an integer in the range of 1-4.

Embodiment 8. The multimeric dye of embodiment 1, wherein n is 1, and A is unsubstituted C₁-C₆ alkyl, —(CH₂CH₂—O)ₚCH₂CH₂—, or —(CH₂CH₂Si(R^{A})₂)ₚCH₂CH₂—, or (CH₂CH₂N (R^{A}))ₚCH₂CH₂—, wherein p is an integer in the range of 2-3.

Embodiment 9. The multimeric dye of embodiment 1, wherein n is 2, A has the formula N[(C₁-C₆ alkyl)-]₃, CH[(C₁-C₆ alkyl)-]₃, CH[(C₁-C₆ alkyl)-]₃, or C(CH₃)[(C₁-C₆ alkyl)-]₃.

Embodiment 10. The multimeric dye of any of embodiments 2-3, wherein A is a ring system of formula Y—X—(—Y)ₙ, in which each Y is independently a monocyclic cycloalkyl, heterocycloalkyl, aromatic, or heteroaromatic ring.

Embodiment 11. The multimeric dye of embodiment 10, wherein Y is cyclohexyl, phenyl, triazolyl, or pyridyl.

Embodiment 12. The multimeric dye of embodiment 10 or embodiment 11, wherein n is 1, and X is a bond, —CH₂—, —C(CH₃)₂—, —C(CF₃)₂—, —O—, —N(H)—, —Si(Me)₂—, —S(O)₀₋₂—, —CH₂—CH₂—, —C(CH₃)₂C(CH₃)₂—, or —C(CF₃)₂C(CF₃)₂—.

Embodiment 13. The multimeric dye of embodiment 10 or embodiment 11, wherein, n is 2, and X is CH, C(CH₃), C(CF₃), or N.

Embodiment 14. The multimeric dye of any of embodiments 1-4, wherein, A is monocylic, a fused bicyclic or a fused tricyclic ring system.

Embodiment 15. The multimeric dye of embodiment 14, wherein A is comprised of one or more aryl rings.

Embodiment 16. The multimeric dye of embodiment 14, wherein A is cyclohexyl, phenyl, benzyl, pyridinyl, triazinyl, naphthyl, tetrahydonaphthyl, fluorenyl, or carbazolyl.

Embodiment 17. The multimeric dye of any of embodiments 14-16, wherein A is substituted by 1-3 R^{A}, where R^{A} is —CH₃, —OCH₃, —F, —Cl, or —CF₃.

Embodiment 18. The multimeric dye of any of embodiments 1-3, wherein, A is comprised of n+2 monocyclic rings joined by n+1 bridging groups X.

Embodiment 19. The multimeric dye of embodiment 18, wherein the monocyclic rings are each independently cyclohexyl, phenyl, fluorenyl, or triazinyl (e.g., are phenyl).

Embodiment 20. The multimeric dye of embodiment 18 or embodiment 19, wherein the monocyclic rings are phenyl.

Embodiment 21. The multimeric dye of embodiment 1, wherein n is 1, and A has the structure

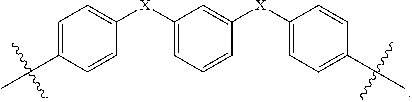

Embodiment 22. The multimeric dye of embodiment 1, wherein n is 2, and A has the structure

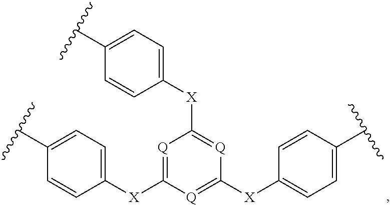

wherein each Q is independently CH or N.

Embodiment 23. The multimeric dye of any of embodiments 18-22, wherein the n+1 bridging groups X are equivalent.

Embodiment 24. The multimeric dye of any of embodiments 18-22, wherein the n+1 bridging groups X are inequivalent.

Embodiment 25. The multimeric dye of any of embodiments 18-24, wherein each X is independently selected from the group consisting of a bond, —O—, —CH₂—, —C(CH₃)₂—, and —C(CF₃)₂—.

Embodiment 26. The multimeric dye of embodiment 1, wherein n is 1, and A comprises four monocyclic rings joined by three bridging groups X, wherein each X is independently —O—, —CH₂—, —C(CH₃)₂—, —Si(Me)₂—, —S(O)₀₋₂—, or —C(CF₃)₂—.

Embodiment 27. The multimeric dye of embodiment 27, wherein the monocyclic rings are phenyl rings.

Embodiment 28. The multimeric dye of embodiment 1, wherein when n is 1, and A comprises a polysiloxane, wherein each Si is optionally substituted with either 2 methyl groups or 2 phenyl groups, and comprises no more than 6 Si atoms (e.g., comprises 2-6 Si atoms).

Embodiment 29. The multimeric dye of embodiment 1, wherein n is 3, and A is C—[(C₁-C₆ alkyl)-]₄ or [—(C₁-C₆ alkyl)₂]N—(C₂-C₆ alkyl)-N—(C₁-C₆ alkyl)-]₂.

Embodiment 30. The multimeric dye of embodiment 29, wherein each alkyl is linear or branched.

Embodiment 31. The multimeric dye of embodiment 1, wherein n is 3, and A is a ring system comprising:
two monocyclic rings joined by one bridging group X or a moiety X—Z—X, wherein each monocyclic ring is bound to two L, if present, or, if L is absent, two D; or
four monocyclic rings joined by one bridging group X or a moiety X—Z—X, wherein each monocyclic ring is bound to one L, if present, or, if L is absent, one D;
wherein Z is a ring system comprising one monocyclic ring or a fused or spiro bicyclic ring.

Embodiment 32. The multimeric dye of embodiment 31, wherein the monocyclic rings are cyclohexyl, phenyl, pyridyl, triazinyl, dioxanyl, or triazolyl (e.g., phenyl).

Embodiment 33. The multimeric dye of embodiment 31 or embodiment 32, wherein each X is independently a bond, —O—, —CH₂—, —C(CH₃)₂—, —S(O)₀₋₂—, or —C(CF₃)₂—.

Embodiment 34. The multimeric dye of embodiment 31 or embodiment 32, wherein each X is independently-O— or —CH₂—.

Embodiment 35. The multimeric dye of any of embodiments 1-34, wherein each L is independently absent, C₁-C₁₀ alkyl, C₂-C₁₀ alkenyl, —O—(C₁-C₈ alkyl)-O₀₋₁; —O—, —S—, —N(R^{A})—, —NHC(O)—, —C(O)O—, —C(O)—, —C(O)N(R^{A})(C₁₋C₄ alkyl)-, —(C₁-C₄ alkyl)N(R^{A})C(O)—; —(C₁-C₃ alkyl)-C(O)N(R^{A})(C₁-C₃ alkyl);
wherein
each alkylene, alkenylene, cycloalkyl, heterocycloalkyl, aryl, and heteroaryl is independently and substituted with 0-5 R^{A}
R^{A} and R^{B} are each independently selected in each occurrence from H, methyl, ethyl, propyl, F, Cl, Br, oxo where chemically appropriate, —CF₃, or —OCH₃.

Embodiment 36. The multimeric dye of any of embodiments 1-34, wherein each L is independently absent, C₁-C₃ alkyl, —(C₁-C₄ alkyl)-O—, —O—, —S—, —N(R^{A})—, —Si(R^{A})₂—, —NHC(O)—, —C(O)O—, —C(O)—, or —(C₁-C₄ alkyl)N(R^{A})C(O)—.

Embodiment 37. The multimeric dye of any of embodiments 1-34, wherein each L is independently absent, C₁-C₃ alkyl, or —O—.

Embodiment 38. The multimeric dye of any of embodiments 1-37, wherein -L-A-(L-)ₙ comprises at least 6 non-hydrogen atoms.

Embodiment 39. The multimeric dye of any of embodiments 1-38 wherein -L-A-(L-)ₙ comprises at least 6 non-hydrogen atoms (e.g., no more than 50 non-hydrogen atoms).

Embodiment 40. The multimeric dye of any of embodiments 1-39, wherein each isolated chromophore D is independently a perylene diimide chromophore, a naphthalene chromophore, a naphthalene monoimide chromophore, a perylene chromophore, an anthraquinone chromophore, a quinone chromophore, a phenazine chromophore, an azo dye chromophore, a triarylmethane chromophore, a transition metal (e.g., chromium) coordination complex chromophore, a cyanine chromophore (e.g., a phthalocyanine chromophore), a phenoxazine chromophore, an indole chromophore, a xanthene chromophore, a coumarin chromophore, a nitro dye chromophore, or an indene chromophore.

Embodiment 41. The multimeric dye of any of embodiments 1-39, wherein each isolated chromophore D is independently a perylene diimide chromophore, a naphthalene chromophore, a naphthalene monoimide chromophore, a perylene chromophore, an anthraquinone chromophore, a quinone chromophore, a phenazine chromophore, or an azo dye chromophore.

Embodiment 42. The multimeric dye of any of embodiments 1-39, wherein each isolated chromophore D is independently a perylene diimide chromophore, or an anthraquinone chromophore.

Embodiment 43. The multimeric dye of any of embodiments 1-42, wherein each isolated chromophore D has a transmittance maximum in the range of 380-450 nm (violet); 450-495 nm (blue); 495-570 nm (green); 570-590 nm (yellow); 590-620 nm (orange); or 620-750 nm (red).

Embodiment 44. The multimeric dye of any of embodiments 1-43, wherein each isolated chromophore D has a molar absorptivity of at least 4000 M⁻¹cm⁻¹, e.g., at least 6000 M⁻¹cm⁻¹ at a wavelength within the 380-750 nm wavelength range.

Embodiment 45. The multimeric dye of any of embodiments 1-44, wherein the multimeric dye has a has a molar absorptivity of at least 4000 M⁻¹cm⁻¹, e.g., at least 8000 M⁻¹cm⁻¹ or at least 15000 M⁻¹cm⁻¹ at a wavelength within the 380-750 nm wavelength range.

Embodiment 46. A colored dielectric polymer material comprising a dielectric polymer and the multimeric dye of any of embodiments 1-45 dispersed in the dielectric polymer.

Embodiment 47. The colored dielectric polymer material of embodiment 46, wherein the multimeric dye is present within in the colored dielectric polymer material in an amount of at least 1 wt %, e.g., at least 3 wt %, at least 10 wt %, or at least 50 wt %.

Embodiment 48. The colored dielectric polymer material of embodiment 46,wherein the multimeric dye is present within in the colored dielectric polymer material in an amount in the range of Jan. 1, 1980 wt % e.g., 1-80 wt %, or 1-50 wt %, or 1-20 wt %, or 3-80 wt %, or 3-50 wt %, or 3-20 wt %, or 3-10 wt %, or 5-80 wt %, or 5-50 wt %, or 5-30 wt %, or 5-20 wt %, or 10-80 wt %, or 10-50 wt %, or 20-80 wt %, or 20-50 wt %.

Embodiment 49. The colored dielectric polymer material of any of embodiments 1-48 wherein the material is provided as a body (e.g., a film) having a transmittance maximum of at least 50% (e.g., at least 75%, at least 90%) at one or more wavelengths in the range of 380-450 nm (violet); 450-495 nm (blue); 495-570 nm (green); 570-590 nm (yellow); 590-620 nm (orange); or 620-750 nm (red).

Embodiment 50. The colored dielectric polymer material of embodiment 49, wherein the body has a transmittance minimum of no more than 20% (e.g., no more than 10%, or no more than 5%) at one or more wavelengths in the range of 380-450 nm (violet); 450-495 nm (blue); 495-570 nm (green); 570-590 nm (yellow); 590-620 nm (orange); or 620-750 nm (red).

Embodiment 51. The colored dielectric polymer material of any of embodiments 1-48, wherein the material is provided as a body (e.g., a film) having a total transmittance of light in wavelength range 380-750 nm of no more than 20%, for example, no more than 10%, no more than 5%, or even no more than 1%.

Embodiment 52. The colored dielectric polymer material of any of embodiments 49-51, wherein the body is no more than 1 mm in thickness, e.g., no more than 100 microns, no more than 50 microns, or even no more than 10 microns in thickness.

Embodiment 53. The colored dielectric polymer material of any of embodiments 49-51, wherein the body has a thickness in the range of 0.05-100 microns, e.g., 0.05-50 microns, or 0.05-10 microns, or 0.05-5 microns, or 0.05-2 microns, or 0.05-1 micron, or 0.1-100 microns, or 0.1-50 microns, or 0.1-10 microns, or 0.1-5 microns, or 0.1-2 microns, or 0.1-1 micron, or 0.2-100 microns, or 0.2-50 microns, or 0.2-10 microns, or 0.2-5 microns, or 0.2-2 microns, or 0.2-1 micron.

Embodiment 54. The colored dielectric polymer material of any of embodiments 1-53, wherein the colored dielectric polymer material does not comprise a pigment.

Embodiment 55. The colored dielectric polymer material of any of embodiments 1-54, having a dielectric constant of 6 or less, e.g., 5 or less, or 4 or less, or 3 or less, or in the range of 2-6, or 2-5, or 2-4, or 2-3 at 1 MHz.

Embodiment 56. The colored dielectric polymer material of any of embodiments 1-54, having a dielectric constant in the range of 2-6, e.g., 2-5, or 2-4, or 2-3 at 1 MHz.

Embodiment 57. The colored dielectric polymer material of any of embodiments 1-56, wherein the colored dielectric polymer material has a dielectric strength (breakdown field) no less than 1 MV/cm (e.g., at least 1 MV/cm, or 1.5 MV/cm, or 2 MV/cm, or 2.5 MV/cm, or 3 MV/cm).

Embodiment 58. The colored dielectric polymer material of any of embodiments 1-57, in the form of a film having a thickness of no more than 4 μm (e.g., no more than 3.5 μm, or no more than 3 μm, or no more than 2.5 μm, or no more than 2 μm, or no more than 1.5 μm).

Embodiment 59. The colored dielectric polymer material of any of embodiments 1-58, in the form of a film having a thickness of at least 50 nm (e.g., at least 100 nm, at least 200 nm, or at least 500 nm).

Embodiment 60. The colored dielectric polymer material of any of embodiments 1-57, in the form of a film having a thickness in the range of 0.05-100 microns, e.g., 0.05-50 microns, or 0.05-10 microns, or 0.05-5 microns, or 0.05-2 microns, or 0.05-1 micron, or 0.1-100 microns, or 0.1-50 microns, or 0.1-10 microns, or 0.1-5 microns, or 0.1-2 microns, or 0.1-1 micron, or 0.2-100 microns, or 0.2-50 microns, or 0.2-10 microns, or 0.2-5 microns, or 0.2-2 microns, or 0.2-1 micron.

Embodiment 61. A colored dielectric polymer material according to any of embodiments 1-60, wherein the crosslinked polymer has a leakage current density of no more than 1×10⁻⁸ A/cm² at an electric field of 1.0 MV/cm.

Embodiment 62. A device comprising a film of the colored dielectric polymer material of any of embodiments 1-61, optionally in contact with a transparent conducting oxide film.

Embodiment 63. The device of embodiment 62, wherein the colored dielectric polymer material is present as a film having a thickness of no more than 4 μm (e.g., no more than 3.5 μm, or no more than 3 μm, or no more than 2.5 μm, or no more than 2 μm, or no more than 1.5 μm).

Embodiment 64. The device of embodiment 62 or embodiment 63, wherein the colored dielectric polymer material is present as a film having a thickness of at least 50 nm (e.g., at least 100 nm, or at least 200 nm, or at least 500 nm).

Embodiment 65. The device of any of embodiments 62-64, wherein the film of the colored dielectric polymer material is prepared through spin-coating, slit-coating, slot-die, or blade coating followed by crosslinking with radiation or thermal exposure.

Embodiment 66. The device of any of embodiments 62-65, wherein the transparent conducting oxide electrode is deposited on the colored dielectric polymer material film through spluttering followed by annealing.

Embodiment 67. The device of embodiment 65, wherein the maximum transmittance of the colored dielectric polymer material after sputtering and annealing is within 20% of the maximum transmittance prior to sputtering and annealing.

Embodiment 68. The device of any of embodiments 62-67, in the form of a liquid crystal cell, the liquid crystal cell comprising:
a first cell plate having a top surface, the first cell plate comprising
a first transparent substrate,
disposed on the first transparent substrate, the colored dielectric polymer material; and
disposed on the colored dielectric polymer material, a first transparent conducting oxide film, the transparent conducing oxide being within 100 nm of the top surface of the first cell plate;
a second cell plate having a top surface, the second cell plate comprising a second transparent substrate,
disposed on the second transparent substrate, a second transparent conducting oxide film, the transparent conducing oxide being within 100 nm of the top surface of the second cell plate;
one or more spacers disposed between the top surface of the first cell plate and the top surface of the second cell plate, the one or more spacers defining lateral edges of the liquid crystal cell; and
a liquid crystal material disposed in a volume defined by the top surface of the first cell plate, the top surface of the second cell plate, and the one or more spacers.

Embodiment 69. The device of any of embodiments 62-67, in the form of a device configured to provide colored light, the device comprising the colored dielectric polymer material operatively coupled to a light source, configured to filter light emanating from the light source in a display direction.

Embodiment 70. A method of making a device according of any of embodiments 62-68, comprising:
forming a film of the colored dielectric polymer material;
depositing a transparent conducting oxide electrode adjacent the film through sputtering; and
annealing at a temperature of at least 200° C. for a time of at least 10 minutes (e.g., up to 24 hours).

1. A multimeric dye having the structure: 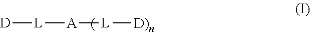
wherein:
each D is independently in isolated chromophore;
each L is independently absent or is a linking group that is no more than 10 atoms in length as measured in the shortest path from D to A;
A is a bridging group; and
n is an integer in the range of 1 to 3.

2. The multimeric dye of claim 1, wherein
A is:
C₁-C₁₀ alkylene or C₂-C₁₀ alkenylene, wherein optionally 1-6 carbon atoms of the backbone of each alkylene and/or alkenylene are each independently replaced by O, N(R^{A})₀₋₁, Si(R^{A})₀₋₂, or S;
monocyclic or spiro and/or fused polycyclic ring system, wherein the ring system comprises cycloalkyl, heterocycloalkyl, aromatic, and/or heteroaromatic rings, and wherein the ring system comprises no more than five total rings, and wherein the ring system optionally comprises 1-3 internal bridge groups X, wherein each X is independently a bond, —C(R^{A})(R^{B})—, —O—, —N(R^{A})₀₋₁—, —Si(R^{A})₀₋₂—, —S(O)₀₋₂—, or —C(R^{A})(R^{B})—C(R^{A}R^{B})—; or
polysiloxane, wherein each Si is optionally substituted with 1-2 methyl groups, or 1-2 phenyl groups, and comprises no more than 10 Si atoms;
wherein
each alkylene, alkenylene, cycloalkyl, heterocycloalkyl, aryl, and heteroaryl is independently and substituted with 0-5 R^{A}; and
R^{A} and R^{B} are each independently selected in each occurrence from H, methyl, ethyl, propyl, F, Cl, Br, oxo where chemically appropriate, —CF₃, or —OCH₃.

3. The multimeric dye of claim 1, wherein n is 1, and A is C₁-C₈ alkylene, —(CH₂CH₂—O)ₚCH₂CH₂—, —(CH₂CH₂Si(R^{A})₂)ₚCH₂CH₂—, or —(CH₂CH₂N(R^{A}))ₚCH₂CH₂—, wherein p is an integer in the range of 1-4.

4. The multimeric dye of claim 1, wherein n is 1, and A has the structure

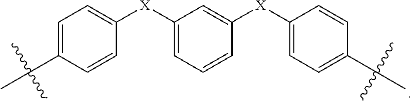

5. The multimeric dye of claim 1, wherein n is 2, and A has the structure 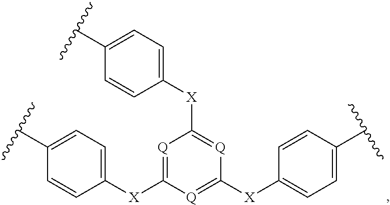
wherein each Q is independently CH or N.

6. The multimeric dye of claim 1, wherein each L is independently absent, C₁-C₁₀ alkyl, C₂-C₁₀ alkenyl, —O—(C₁-C₈ alkyl)-O₀₋₁; —O—, —S—, —N(R^{A})—, —NHC(O)—, —C(O)O—, —C(O)—, —C(O)N(R^{A})(C₁-C₄ alkyl)-, —(C₁-C₄ alkyl)N(R^{A})C(O)—; —(C₁-C₃ alkyl)-C(O)N(R^{A})(C₁-C₃ alkyl);
wherein
each alkylene, alkenylene, cycloalkyl, heterocycloalkyl, aryl, and heteroaryl is independently and substituted with 0-5 R^{A}; and
R^{A} and R^{B} are each independently selected in each occurrence from H, methyl, ethyl, propyl, F, Cl, Br, oxo where chemically appropriate, —CF₃, or —OCH₃.

7. The multimeric dye of claim 1, wherein each isolated chromophore D is independently a perylene diimide chromophore, a naphthalene chromophore, a naphthalene monoimide chromophore, a perylene chromophore, an anthraquinone chromophore, a quinone chromophore, a phenazine chromophore, an azo dye chromophore, a triarylmethane chromophore, a transition metal coordination complex chromophore, a cyanine chromophore, a phenoxazine chromophore, an indole chromophore, a xanthene chromophore, a coumarin chromophore, a nitro dye chromophore, or an indene chromophore.

8. The multimeric dye of claim 1, wherein each isolated chromophore D has a transmittance maximum in the range of 380-450 nm (violet); 450-495 nm (blue); 495-570 nm (green); 570-590 nm (yellow); 590-620 nm (orange); or 620-750 nm (red).

9. The multimeric dye of claim 1, wherein each isolated chromophore D has a molar absorptivity of at least 4000 M⁻¹cm⁻¹ at a wavelength within the 380-750 nm wavelength range.

10. A colored dielectric polymer material comprising the multimeric dye of claim 1, wherein the multimeric dye is present within in the colored dielectric polymer material in an amount of at least 1 wt %.

11. The colored dielectric polymer material of claim 10, wherein the colored dielectric polymer material is provided as a body having a transmittance maximum of at least 50% at one or more wavelengths in the range of 380-450 nm (violet); 450-495 nm (blue); 495-570 nm (green); 570-590 nm (yellow); 590-620 nm (orange); or 620-750 nm (red).

12. The colored dielectric polymer material of claim 11, wherein the body has a transmittance minimum of no more than 20% at one or more wavelengths in the range of 380-450 nm (violet); 450-495 nm (blue); 495-570 nm (green); 570-590 nm (yellow); 590-620 nm (orange); or 620-750 nm (red).

13. The colored dielectric polymer material of claim 10, having a dielectric constant of 6 or less.

14. The colored dielectric polymer material of claim 10, wherein the colored dielectric polymer material has a dielectric strength (breakdown field) no less than 1 MV/cm.

15. The colored dielectric polymer material of claim 10, in the form of a film having a thickness of no more than 4 μm.

16. A device comprising a film of the colored dielectric polymer material of claim 10.

17. The device of claim 16, wherein the maximum transmittance of the colored dielectric polymer material after sputtering and annealing is within 20% of the maximum transmittance prior to sputtering and annealing.

18. The device of claim 16, in the form of a liquid crystal cell, the liquid crystal cell comprising:
a first cell plate having a top surface, the first cell plate comprising
a first transparent substrate,
disposed on the first transparent substrate, the colored dielectric polymer material; and
disposed on the colored dielectric polymer material, a first transparent conducting oxide film, the transparent conducing oxide being within 100 nm of the top surface of the first cell plate;
a second cell plate having a top surface, the second cell plate comprising
a second transparent substrate,
disposed on the second transparent substrate, a second transparent conducting oxide film, the transparent conducing oxide being within 100 nm of the top surface of the second cell plate;
one or more spacers disposed between the top surface of the first cell plate and the top surface of the second cell plate, the one or more spacers defining lateral edges of the liquid crystal cell; and
a liquid crystal material disposed in a volume defined by the top surface of the first cell plate, the top surface of the second cell plate, and the one or more spacers.
